(19) United States
(12) Patent Application Publication
(10) Pub. No.: US 2024/0257907 A1
(43) Pub. Date: Aug. 1, 2024

(54) SYSTEMS AND METHODS FOR IDENTIFYING MUTANTS

(71) Applicant: BioMetis Technology, Inc., Hayward, CA (US)

(72) Inventors: Jie Yang, Foster City, CA (US); Xiyun Zhang, San Ramon, CA (US); Imad N. Sawaya, Pleasanton, CA (US)

(73) Assignee: BioMetis Technology, Inc., Hayward, CA (US)

(21) Appl. No.: 18/017,525

(22) PCT Filed: Jan. 20, 2023
(86) PCT No.: PCT/US2023/011194

§ 371 Date: Jan. 23, 2023

Related U.S. Application Data
(60) Provisional application No. 63/301,443, filed on Jan. 20, 2022.

(51) Int. Cl. G16B 20/50; G06N 3/044; G06N 3/0464; G06N 7/01; G06N 20/10; G16B 40/20

(52) U.S. Cl. CPC G16B 20/50; G06N 3/044; G06N 3/0464; G06N 7/01; G06N 20/10; G16B 40/20

(57) ABSTRACT

Systems and methods improving a target property of a target protein are provided. Each single point mutation in a first plurality of single point mutations of the target protein is obtained, each defining a corresponding single point substituted protein with respect to a reference sequence. A set of values for a set of properties of each point substituted protein is used to filter the first plurality of single point mutations into a reduced second plurality of single point mutations. The second plurality of mutations informs the selection of combinatorially substituted proteins and the target property is measured for each of them. The combinatorially substituted proteins and their measured values serve to train a surrogate model that, in turn, serves to update a search model. The updated search mode informs on which of the second plurality point mutations are to be used in future mutants of the target protein.

### TECHNICAL FIELD

The present invention is directed to system and methods for directing protein evolution of a target protein in order to improve one or more properties of the target protein.

### BACKGROUND

Proteins are large biomacromolecules that include long chains of amino acid residues. Proteins can perform many different functions and are widely used in many bioindustrial and pharmaceutical applications. Protein engineering and directed evolution are proven approaches to improving protein's stability, activity, substrate selectivity and other properties that affect its feasibility in bioindustrial and pharmaceutical applications. Artificial Intelligence (AI), powered by advanced computational hardware, state-of-the-art algorithms, and big data, revolutionizes humankind's everyday life. Significant progress has been made in applying AI in image recognition and language processing.

In 2021, accurate protein structure prediction was achieved with AlphaFold. See Jumper et al., 2021, “Highly accurate protein structure prediction with AlphaFold,” Nature 596, pp. 583-592. AlphaFold is a computational approach that incorporates physical and biological information, distilled from numerous protein structures, and evolutionary relationships, extracted from multi-sequence alignments, into the design of deep learning algorithms.

Despite the breakthrough in AI-directed protein structure prediction, AI-directed protein evolution is still in its infancy and faces at least five significant challenges.

First, protein evolution is a multi-parameter optimization problem. Usually, single residue beneficial mutations only marginally improve one protein property, yet single residue deleterious mutations can cause substantial loss of protein function. Thus, removing the single residue deleterious mutations before combining neutral and beneficial single residue mutations is desired. However, it is costly to evaluate different functions of a large set of single residue mutations experimentally.

Second, while incorporating a plethora of neutral and beneficial single residue mutations is a suitable approach to expediting protein evolution, conventional combinatorial library construction methods can only target a limited number of single residue mutations in a limited number of regions of the protein. This leads to many drawbacks. One draw back is that iterative directed evolution is required to obtain a final candidate that meets various desired criteria. Although proven successful, iterative directed evolution is expensive, time-consuming, and path dependent. Another drawback is that typical protein evolution data often contains results for mutants with a very narrow mutation rate (e.g., 1-10 mutations per mutant and centered at around 1-3 mutations per mutant). This limited mutation rate range does not allow sufficient consideration of interactions between different residues. Therefore, linear methods are commonly used to acquire a working model that provides direction for protein evolution.

Third, how to combine many single residue mutations is an NP-hard problem, indicating it is complicated to search for the shortest path to the best functional protein due to combinatorial explosion.

Fourth, AI inference will lead to a large number of combined mutants with comparable functions considering the model variations. Painstaking efforts are required to prioritize a limited number of mutants to be synthesized and evaluated experimentally.

Fifth, it is well-known that, although AI can be precise in making inferences for data within the same distribution as the original data set, it cannot infer new data that is out-of-distribution of the original data set.

Because of the above-identified drawbacks, conventional iterative protein evolution tends to take numerous rounds before acceptable convergence (e.g., can take 8-15 iterative rounds). See Cobb et al., 2013, “Directed evolution: past, present and future,” AIChE J. 59(5), pp. 1432-1440.

Given the above-background, what is needed in the art are improved methods for directing protein evolution of a target protein in order to improve one or more properties of the target protein.

### SUMMARY

The present disclosure addresses the shortcomings disclosed above by providing systems and methods that integrate: 1) library design using various in silico detection methods to measure specifics of single mutations and rule out single deleterious mutations: 2) intelligent library construction that makes mutants with a wide range of mutation rates and allows the deep interactions of different mutations: 3) library screening that generates a diverse set of functional data for the mutants: 4) library mutation-function results that encode an effective learning to acquire a surrogate model without over-fitting: 5) optimal library design using a search model to guide the selection of single residue mutations and mutation rate range; and 6) construction and screening of the optimal library to obtain the protein candidates with improved properties. Using the disclosed methodology, desired properties of target proteins are realized in as few as two rounds of evolution.

Turning to more specific details, an aspect of the present disclosure is directed to providing a computer system for identifying one or more combinatorial substitutions that affect one or more properties of a target protein. The computer system includes one or more processors, a memory, and one or more programs. The one or more programs are stored in the memory and are configured to be executed by the one or more processors. The one or more programs identify one or more combinatorial substitutions that affect a first property of a target protein. Accordingly, the one or more programs include instructions for obtaining an identity of each single point mutation in a first plurality of single point mutations of the target protein. Each respective single point mutation in the first plurality of single point mutations defines a corresponding single point substituted protein characterized by a reference sequence for the target protein with the exception of an alteration at a respective independent position within the reference sequence to an amino acid other than that found in the reference sequence.

The one or more programs further include instructions for obtaining a corresponding set of values for a set of properties of the corresponding point substituted protein for each corresponding point substituted protein defined by the first plurality of single point mutations. The set of properties includes a stability of the corresponding point substituted protein, at least one protein formulation property of the corresponding point substituted protein, and a determination that the respective single point mutation in the corresponding point substituted protein occurs at a predetermined position that exhibits variability across a plurality of naturally occurring homologs of the target protein. Moreover, the one or more programs includes instructions for filtering the first plurality of single point mutations to form a second plurality of single point mutations. This filtering is based at least upon each corresponding set of values for the set of properties. Furthermore, the filtering includes determining, for each corresponding point substituted protein defined by the first plurality of single point mutations, for each respective property in the set of properties, whether a value of the respective property in the corresponding set of values for the corresponding point substituted protein satisfies a corresponding threshold value requirement for the respective property. The corresponding point substituted protein is included in the second plurality of single point mutations when each corresponding threshold value requirement for each property in the set of properties is satisfied. Moreover, the corresponding point substituted protein is not included in the second plurality of single point mutations when any corresponding threshold value requirement of any property in the set of properties is not satisfied. Additionally, the one or more programs includes instructions for obtaining a corresponding measured value of the first property for each combinatorially substituted protein in a first plurality of combinatorially substituted proteins. Each respective combinatorially substituted protein in the first plurality of combinatorially substituted proteins is characterized by the reference sequence for the target protein with the exception of the independent inclusion of two or more single point mutations from the second plurality of single point mutations.

The one or more programs further include instructions for training a surrogate model within an N-dimensional space, in which N is a positive integer of ten or greater. This training the surrogate model uses at least the corresponding measured value of the first property in the respective combinatorially substituted proteins against an identity of each single point mutation in the respective combinatorially substituted protein for each respective combinatorially substituted protein in the first plurality of combinatorially substituted proteins. Moreover, the model includes 20 or more parameters and the first plurality of combinatorially substituted proteins includes 20 or more proteins.

The one or more programs further include instructions for using the surrogate model and the identity of each single point mutation in the respective combinatorially substituted protein for each respective combinatorially substituted protein in the first plurality of combinatorially substituted proteins to update a search model. Furthermore, the one or more programs includes instructions for using the updated search model to identify a second plurality of combinatorially substituted proteins within the N-dimensional space. Each respective combinatorially substituted protein in the second plurality of combinatorially substituted proteins is characterized by the reference sequence for the target protein with the exception of independent inclusion of two or more single point mutations from the second plurality of single point mutations.

In some embodiments, the at least one protein formulation property is an electrostatic property of the corresponding point substituted protein, a developability index of the corresponding point substituted protein, a solubility of the corresponding point substituted protein, a measure of aggregation of the corresponding point substituted protein, a viscosity of the corresponding point substituted protein, or a combination thereof.

In some embodiments, the using the updated search model identifies an optimal range of single point mutations, drawn from the second plurality of single point mutations to incorporate into the target protein.

In some embodiments, the set of properties further includes a post-translational modification that is predicted to occur to the corresponding point substituted protein.

In some embodiments, the set of properties further includes an immunogenicity of the corresponding point substituted protein.

In some embodiments, the set of properties further includes a binding energy of the corresponding point substituted protein.

In some embodiments, the first property of the target protein is a solubility of the target protein, an ability of the target protein to carry out an enzymatic activity in a predetermined pH range, aliphatic index, a molecular weight of the target protein, or a charge of the of the target protein.

In some embodiments, each combinatorially substituted protein in the first plurality of combinatorially substituted proteins includes three or more, four or more, five or more, or six or more point substitutions.

In some embodiments, each combinatorially substituted protein in the first plurality of combinatorially substituted proteins includes between three and fifty point substitutions.

In some embodiments, the target protein is an enzyme and the first property is an enzymatic activity of the target protein.

In some embodiments, the enzyme is a hydrolase, oxidoreductase, lyase, transferase, ligase or isomerase.

In some embodiments, the target protein includes 50 or more residues, or 100 or more residues.

In some embodiments, the stability of the corresponding point substituted protein is determined using one or more crystal structures or atomistic models of the target protein.

In some embodiments, the corresponding threshold value for the stability is a stability of the target protein. When the corresponding point substituted protein has a stability that is better than the stability of the target protein, the corresponding point substituted protein is included in the second plurality of single point mutations. Moreover, when the corresponding point substituted protein has a stability that is worse than the stability of the target protein, the corresponding point substituted protein is not included in the second plurality of single point mutations.

In some embodiments, the corresponding threshold value for the stability is a stability of the target protein. When the corresponding point substituted protein has a stability that is at least a threshold percentage or better than the stability of the target protein, the corresponding point substituted protein is included in the second plurality of single point mutations. Furthermore, when the corresponding point substituted protein has a stability that is less than a threshold percentage of the stability of the target protein, the corresponding point substituted protein is not included in the second plurality of single point mutations.

In some embodiments, the training the surrogate model includes encoding each respective combinatorially substituted protein in the first plurality of combinatorially substituted proteins as an identity of each single point mutation in the respective combinatorially substituted protein in a first dimension, and a position of each single point mutation in the respective combinatorially substituted protein in a second dimension.

In some embodiments, the training the surrogate model includes encoding each respective combinatorially substituted protein in the first plurality of combinatorially substituted proteins as an identity of each single point mutation in the respective combinatorially substituted protein in a first dimension, a position of each single point mutation in the respective combinatorially substituted protein in a second dimension, and a plurality of amino acid indices, or a low dimension or latent dimension thereof, for each of the naturally occurring amino acids in a third dimension.

In some embodiments, the surrogate model is a support Vector Regression with RBF kernel, a random forest, XGBoost, a Gaussian Process, a deep neural network, a convolutional neural network, or a recurrent neural network.

In some embodiments, the target protein is an enzyme, a co-enzyme, a structural protein, a nutrient protein, a regulatory protein, a defense protein, a transport protein, a storage protein, a contractile protein, or a toxic protein.

In some embodiments, the using the updated search model identifies optimal single point mutations in the second plurality of single point mutations to incorporate into the target protein.

In some embodiments, the using the updated search model rank orders each single point mutation in the second plurality of single point mutations to incorporate into the target protein.

Another aspect of the present disclosure is directed to providing a non-transitory computer-readable storage medium. The non-transitory readable storage medium includes instructions, which when executed by an electronic device, with one or more processors and a memory, cause the electronic device to identify one or more combinatorial substitutions that affect a first property of a target protein by a method. The method includes obtaining an identity of each single point mutation in a first plurality of single point mutations of the target protein. Each respective single point mutation in the first plurality of single point mutations defines a corresponding single point substituted protein characterized by a reference sequence for the target protein with the exception of an alteration at a respective independent position within the reference sequence to an amino acid other than that found in the reference sequence. The method further includes obtaining a corresponding set of values for a set of properties of the corresponding point substituted protein for each corresponding point substituted protein defined by the first plurality of single point mutations. The set of properties includes a stability of the corresponding point substituted protein, at least one protein formulation property of the corresponding point substituted protein, and a determination that the respective single point mutation in the corresponding point substituted protein occurs at a predetermined position that exhibits variability across a plurality of naturally occurring homologs of the target protein. Moreover, the method includes filtering the first plurality of single point mutations to form a second plurality of single point mutations. This filtering is based at least upon each corresponding set of values for the set of properties. Furthermore, the filtering includes determining, for each corresponding point substituted protein defined by the first plurality of single point mutations, for each respective property in the set of properties, whether a value of the respective property in the corresponding set of values for the corresponding point substituted protein satisfies a corresponding threshold value requirement for the respective property. The corresponding point substituted protein is included in the second plurality of single point mutations when each corresponding threshold value requirement for each property in the set of properties is satisfied. Moreover, the corresponding point substituted protein is not included in the second plurality of single point mutations when any corresponding threshold value requirement of any property in the set of properties is not satisfied. Additionally, the method includes obtaining a corresponding measured value of the first property for each combinatorially substituted protein in a first plurality of combinatorially substituted proteins. Each respective combinatorially substituted protein in the first plurality of combinatorially substituted proteins is characterized by the reference sequence for the target protein with the exception of the independent inclusion of two or more single point mutations from the second plurality of single point mutations. The method includes training a surrogate model within an N-dimensional space, in which N is a positive integer of 10 or greater. This training the surrogate model uses at least the corresponding measured value of the first property in the respective combinatorially substituted proteins against an identity of each single point mutation in the respective combinatorially substituted protein for each respective combinatorially substituted protein in the first plurality of combinatorially substituted proteins. Moreover, the model includes 20 or more parameters and the first plurality of combinatorially substituted proteins includes 20 or more proteins. The method includes using the surrogate model and the identity of each single point mutation in the respective combinatorially substituted protein for each respective combinatorially substituted protein in the first plurality of combinatorially substituted proteins to update a search model. Furthermore, the method includes using the updated search model to identify a second plurality of combinatorially substituted proteins within the N-dimensional space. Each respective combinatorially substituted protein in the second plurality of combinatorially substituted proteins is characterized by the reference sequence for the target protein with the exception of independent inclusion of two or more single point mutations from the second plurality of single point mutations.

Yet another aspect of the present disclosure is directed to providing a method for identifying one or more combinatorial substitutions that affect a first property of a target protein. The method includes obtaining an identity of each single point mutation in a first plurality of single point mutations of the target protein. Each respective single point mutation in the first plurality of single point mutations defines a corresponding single point substituted protein characterized by a reference sequence for the target protein with the exception of an alteration at a respective independent position within the reference sequence to an amino acid other than that found in the reference sequence. The method further includes obtaining a corresponding set of values for a set of properties of the corresponding point substituted protein for each corresponding point substituted protein defined by the first plurality of single point mutations. The set of properties includes a stability of the corresponding point substituted protein, at least one protein formulation property of the corresponding point substituted protein, and a determination that the respective single point mutation in the corresponding point substituted protein occurs at a predetermined position that exhibits variability across a plurality of naturally occurring homologs of the target protein. Moreover, the method includes filtering the first plurality of single point mutations to form a second plurality of single point mutations. This filtering is based at least upon each corresponding set of values for the set of properties. Furthermore, the filtering includes determining, for each corresponding point substituted protein defined by the first plurality of single point mutations, for each respective property in the set of properties, whether a value of the respective property in the corresponding set of values for the corresponding point substituted protein satisfies a corresponding threshold value requirement for the respective property. The corresponding point substituted protein is included in the second plurality of single point mutations when each corresponding threshold value requirement for each property in the set of properties is satisfied. Moreover, the corresponding point substituted protein is not included in the second plurality of single point mutations when any corresponding threshold value requirement of any property in the set of properties is not satisfied. Additionally, the method includes obtaining a corresponding measured value of the first property for each combinatorially substituted protein in a first plurality of combinatorially substituted proteins. Each respective combinatorially substituted protein in the first plurality of combinatorially substituted proteins is characterized by the reference sequence for the target protein with the exception of the independent inclusion of two or more single point mutations from the second plurality of single point mutations. The method includes training a surrogate model within an N-dimensional space, in which N is a positive integer of 10 or greater. This training the surrogate model uses at least the corresponding measured value of the first property in the respective combinatorially substituted proteins against an identity of each single point mutation in the respective combinatorially substituted protein for each respective combinatorially substituted protein in the first plurality of combinatorially substituted proteins. Moreover, the model includes 20 or more parameters and the first plurality of combinatorially substituted proteins includes 20 or more proteins. The method includes using the surrogate model and the identity of each single point mutation in the respective combinatorially substituted protein for each respective combinatorially substituted protein in the first plurality of combinatorially substituted proteins to update a search model. Furthermore, the method includes using the updated search model to identify a second plurality of combinatorially substituted proteins within the N-dimensional space. Each respective combinatorially substituted protein in the second plurality of combinatorially substituted proteins is characterized by the reference sequence for the target protein with the exception of independent inclusion of two or more single point mutations from the second plurality of single point mutations.

### BRIEF DESCRIPTION OF THE DRAWINGS

FIGS. 1A and 1B collectively illustrate a computer system for identifying one or more combinatorial substitutions that affect one or more properties of a target protein, in accordance with an embodiment of the present disclosure:

FIGS. 2A, 2B, 2C, 2D, and 2E collectively provide a flow chart illustrating exemplary methods for identifying one or more combinatorial substitutions that affect one or more property of a target protein, in which dashed boxes indicate optional features, in accordance with some embodiments of the present disclosure:

FIG. 3 illustrates a calculated mutation energy (e.g., a first property in a set of properties that includes a stability of the corresponding point substituted protein) for Beneficial-Neutral (B-N) and Deleterious (D) groups of 1140 single mutants of Neocallimastix patriciarum xylanase, whereby B-N or D is determined experimentally at pH 2.5 at 37° C., in comparison to a wild type enzyme, in accordance with an embodiment of the present disclosure:

FIG. 4 illustrates protein function (PF) of 22 single point mutants of Aspergillus udagawae endoglucanase determined experimentally using a CMC assay at a pH 6.5, and a temperature of 50° C., in accordance with an embodiment of the present disclosure:

FIG. 5 illustrates a relationship between mutation rate and function and evaluation of interactions between single point mutations of Aspergillus udagawae endoglucanase, in accordance with an embodiment of the present disclosure:

FIG. 6A illustrates an epistatic interaction map for top mutants with PF=2.3, whereby each node represents a mutation, darker color indicates higher (more favorable) PF, and whereby edges represents epistatic interactions and labels are calculated AED values for the respective interaction in accordance with an embodiment of the present disclosure:

FIG. 6B illustrates another epistatic interaction map for top mutants with PF=2.3, whereby each node represents a mutation, darker color indicates higher (more favorable) PF, and whereby edges represents epistatic interactions and labels are calculated AED values for the respective interaction, in accordance with an embodiment of the present disclosure:

FIG. 6C illustrates yet another epistatic interaction map for top mutants with PF=2.3, whereby each node represents a mutation, darker color indicates higher (more favorable) PF, and whereby edges represents epistatic interactions and labels are calculated AED values for the respective interaction, in accordance with an embodiment of the present disclosure:

FIG. 6D illustrates yet another epistatic interaction map for top mutants with PF=2.3, whereby each node represents a mutation, darker color indicates higher (more favorable) PF, and whereby edges represents epistatic interactions and labels are calculated AED values for the respective interaction, in accordance with an embodiment of the present disclosure:

FIG. 7A illustrates an encoding process, in accordance with an embodiment of the present disclosure:

FIG. 7B illustrates aspects of a first process in a learning stage, in accordance with an embodiment of the present disclosure:

FIGS. 7C, 7D, and 7E collectively illustrate a second process in a learning stage, in accordance with an embodiment of the present disclosure:

FIG. 7F illustrates a third process in a learning stage, in accordance with an embodiment of the present disclosure:

FIGS. 8A, 8B, 8C, and 8D collectively illustrate a comparison of results between Chaetomium thermophilum endoglucanase library of the present disclosure and three conventional libraries, in accordance with an embodiment of the present disclosure:

FIGS. 9A, 9B, 9C, 9D, and 9E collectively illustrate results provided by a model (e.g., surrogate model and/or search model) for a first protein of Aspergillus udagawae endoglucanase:

FIGS. 10A and 10B collectively illustrate results of an experimental validation provided by a model (e.g., surrogate model and/or search model) for inferred mutants of Chaetomium thermophilum endoglucanase:

FIG. 11A illustrates a two-dimensional encoding matrix, in accordance with an embodiment of the present disclosure;

FIG. 11B illustrates another two-dimensional encoding matrix, in accordance with an embodiment of the present disclosure:

FIG. 12A illustrates the function of Top 10 Mutants from Lib1 at pH 4.5 and 60° C.:

FIG. 12B illustrates the function of Top 10 Mutants from Lib2 at pH 4.5 and 60° C.:

FIG. 12C illustrates the function of Top 4 Mutants at different conditions:

FIG. 13 illustrates the function (PF) of 22 single mutants of Aspergillus udagawae endoglucanase determined experimentally using a CMC assay at pH 4.5 and 62° C.:

FIG. 14A illustrates the mutation frequency of Aspergillus udagawae endoglucanase protein library comprising 22 mutations:

FIG. 14B illustrates the mutation rate of Aspergillus udagawae endoglucanase protein library comprising 22 mutations;

FIG. 15 illustrates the relationship between mutation rate and function (PF) of mutants from the one pot intelligent library of Aspergillus udagawae endoglucanase:

FIG. 16A illustrates the mutation frequency of Chaetomium thermophilum endoglucanase protein library comprising 50 mutations;

FIG. 16B illustrates the mutation rate of Chaetomium thermophilum endoglucanase protein library comprising 50 mutations:

FIG. 17 illustrates the relationship between mutation rate and function (PF) of Chaetomium thermophilum endoglucanase:

FIG. 18 illustrates the surrogate model obtained from learning Aspergillus udagawae endoglucanase protein library using CNN:

FIG. 19 illustrates the results from the surrogate and search models of Aspergillus udagawae endoglucanase activity at pH 4.5 and 62° C.:

FIG. 20A illustrates the results from HTP screening of Aspergillus udagawae endoglucanase mutant libraries at pH 4.5 and 62° C.:

FIG. 20B illustrates the function of Top mutants from Aspergillus udagawae endoglucanase 22-Mut Lib and 15-Mut Lib at pH 4.5 and 62° C.:

FIG. 21A illustrates the surrogate model obtained from learning pullulanase protein library Lib1:

FIG. 21B illustrates the results from the surrogate and search models of pullulanase activity at pH 4.5 and 60° C.:

FIG. 21C illustrates the results from HTP screening of pullulanase 35-Mut Optimal Lib at pH 4.5 and 60° C. (IntLib-V1, IntLib-V2, and IntLib-V3 are the Top 3 mutants from the Intelligent Lib1: OptLib-V includes all the mutants from the Optimal Lib: Parent is the pullulanase used to make both libraries); and

FIG. 21D illustrates the function of Top mutants from pullulanase 71-Mut Intelligent Lib and 35-Mut Optimal Lib at pH 4.5 and 60° C. (IntLib-V1, IntLib-V2, and IntLib-V3 are the Top 3 mutants from the Intelligent Lib1: OptLib-V1, OptLib-V2, OptLib-V3, OptLib-V4, OptLib-V5, OptLib-V6, OptLib-V7, OptLib-V8, OptLib-V9, and OptLib-V10 are the Top 10 mutants from the Optimal Lib: Parent is the starting pullulanase).

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

The present disclosure is directed to providing system and methods for identifying one or more combinatorial substitutions that affect one or more properties (e.g., a first property) of a target protein. As a non-limiting example, in some embodiments, the one or more combinatorial substitutions includes a first single point mutation (αXXβ) and a second single point mutation (δYYε) that affect a melting temperature of a first target protein, where XX and YY are each an independent residue position within the first target protein, a and 8 are the amino acid identities of the reference residues (amino acids) at respective positions XX and YY in the first target protein, and β and ε are the point-substituted residues at respective positions XX and YY in the first target protein. In some embodiments, a first property (in the one or more target properties) and/or the first target protein is selected, at least in part, by an administrator of a computer system. Accordingly, some aspects of the systems and methods of the present disclosure are implemented using the computer system. Accordingly, by requiring the computer system, the systems and methods of the present disclosure cannot be mentally performed.

The systems and methods of the present disclosure obtain an identity of each single point mutation in a first plurality of single point mutations of the target protein, such as by way of a data set indicative of each αXXβ, δYYε, . . . , etc.). Accordingly, each respective single point mutation in the first plurality of single point mutations defines a corresponding single point substituted protein characterized by (having) the reference sequence for the target protein with the exception of an alteration at a respective independent position within the reference sequence to an amino acid other than that found in the reference sequence. In other words, each target protein is 100 percent identical to the reference sequence, except for a single residue, e.g., a single αXXβ. In some embodiments, each single point mutation in the first plurality of single point mutations of the target protein is also N-terminally and/or or C-terminally truncated by a common amount (same number of residues) relative to the reference sequence for the target protein. In some embodiments, the reference sequence for the target protein is the naturally occurring amino acid sequence of the target protein. In some embodiments, the reference sequence for the target protein contains any number of mutations, insertions, translocation, or deletions with respect to the naturally occurring sequence of the target protein.

The systems and methods of the present disclosure further obtain a corresponding set of values for a set of properties of the corresponding point substituted protein for each corresponding point substituted protein defined by the first plurality of single point mutations. In some such embodiments, this corresponding set of values is obtained by at least one model (e.g., a first model) in a plurality of models. The set of properties includes a stability, such as a mutation energy stability (ΔΔGₘᵤₜ) of the corresponding point substituted protein. Moreover, the set of properties includes at least one protein formulation property of the corresponding point substituted protein. By way of example, in some embodiments, the at least one protein formulation property includes one or more electrostatic properties of the corresponding point substituted protein including an isoelectric point of the corresponding point substituted protein, a pH of maximal stability of the corresponding point substituted protein, a net charge of the corresponding point substituted protein, a dipole moment of the corresponding point substituted protein, or a combination thereof. In some embodiments, the at least one protein formulation property includes a per-residue aggregation value (e.g., an average of per-atom aggregation propensity values for a respective residue). As another non-limiting example, in some embodiments, the at least one protein formulation property includes a solubility value. As yet another non-limiting example, in some embodiments, the at least one protein formulation property includes a developability index and/or a viscosity of the corresponding point substituted protein. Furthermore, the set of properties includes a determination that the respective single point mutation in the corresponding point substituted protein occurs at a predetermined position that exhibits variability across a plurality of naturally occurring homologs of the target protein. For instance, in some embodiments, this determination includes searching a plurality of homologs (e.g., at least two homologs, at least 10 homologs, at least 50 homologs, at least 250 homologs, etc.) for the target protein and sequence alignment of the target protein and the plurality of homologs to determine a conservation value of each sequence point and the amino acid substitutions that exist naturally. However, the present disclosure is not limited thereto. Moreover, the systems and methods of the present disclosure filter the first plurality of single point mutations to form a second plurality of single point mutations, by removing one or more unwanted (e.g., undesirable) single point mutations from the first plurality of single point mutations. For instance, in some embodiments the filtering of the first plurality of single point mutations to arrive at the second plurality of single point mutations is based at least upon each corresponding set of values for the corresponding set of properties for each corresponding point substituted protein defined by the first plurality of single point mutations. In this way, the filtering includes determining whether a value of the respective property in the corresponding set of values for the corresponding point substituted protein satisfies a corresponding threshold value requirement for the respective property for each corresponding point substituted protein defined by the first plurality of single point mutations, for each respective property in the set of properties. The corresponding point substituted protein is included in the second plurality of single point mutations when each corresponding threshold value requirement for each property in the set of properties is satisfied, such as when a corresponding value of a respective property is greater than or equal to a corresponding threshold value. As a non-limiting example, consider a first property of a protein function (PF), a first point substituted protein G142T of an endoglucase from Aspergillus udagawae that has a first PF value of 1.7 in a corresponding set of values, and a corresponding threshold value that is PF≥0.8. Accordingly, the first PF value satisfies the corresponding threshold value, which allows inclusion of the first point substituted protein G142T within the second plurality of single point mutations. Moreover, the corresponding point substituted protein is not included in the second plurality of single point mutations when any corresponding threshold value requirement of any property in the set of properties is not satisfied (e.g., when a corresponding PF value of a respective point substituted protein is <0.8). Additionally, the systems and methods of the present disclosure obtain a corresponding measured value of the first property for each combinatorially substituted protein in a first plurality of combinatorially substituted proteins. In some embodiments, the corresponding measured value of the first property is obtained by the at least one model (e.g., a second model) in the plurality of models. Each respective combinatorially substituted protein in the first plurality of combinatorially substituted proteins is characterized by the reference sequence for the target protein with the exception of the independent inclusion of two or more single point mutations from the second plurality of single point mutations. Said otherwise, each respective combinatorially substituted protein in the first plurality of combinatorially substituted proteins has the reference sequence of the first protein with the exception of the two or more single point mutations from the second plurality of single point mutations. In some embodiments, combinatorially substituted protein in the first plurality of combinatorially substituted proteins is also N-terminally and/or or C-terminally truncated by a common amount (same number of residues) relative to the reference sequence for the target protein.

The systems and methods of the present disclosure train a surrogate model (e.g., a third model in the plurality of models) within an N-dimensional space. In some embodiments, the surrogate model does not include one or more a priori conditions (e.g., restrictions, rules, parameters), such as concavity or convexity. In some embodiments, the N-dimensional space is a mathematical data set, in which a corresponding point (e.g., data element) in the N-dimensional space is of a respective combinatorially substituted protein in a plurality of combinatorially substituted proteins. In this way, in some embodiments, N is a positive integer of 10 or greater (e.g., N is about 15, N is about 20, N is about 50, N is about 100, N is about 1,000, etc.). The systems and methods of the present disclosure train the surrogate model by using at least the corresponding measured value of the first property in the respective combinatorially substituted proteins against an identity of each single point mutation in the respective combinatorially substituted protein for each respective combinatorially substituted protein in the first plurality of combinatorially substituted proteins. Moreover, the model includes 20 or more parameters and the first plurality of combinatorially substituted proteins includes 20 or more proteins. In this way, the systems and methods of the present disclosure cannot be mentally performed. The systems and methods of the present disclosure use the surrogate model and the identity of each single point mutation in the respective combinatorially substituted protein for each respective combinatorially substituted protein in the first plurality of combinatorially substituted proteins to update a search model (e.g., a fourth model in the plurality of models). In this way, the surrogate model is utilized, at least in part, to update the search model within the context of the N-dimensional space and the first property of the target protein. Furthermore, the systems and methods of the present disclosure use the updated search model to identify a second plurality of combinatorially substituted proteins within the N-dimensional space. Each respective combinatorially substituted protein in the second plurality of combinatorially substituted proteins is characterized by the reference sequence for the target protein with the exception of independent inclusion of two or more single point mutations from the second plurality of single point mutations. Accordingly, the systems and methods of the present disclosure identify the one or more combinatorial substitutions that affect the first property of the target protein using fewer resources, and fewer iterations. This greatly improves both computational efficiency when using the systems and methods of the present disclosure at the computer system since the present disclosure identifies the one or more combinatorial substitutions in fewer rounds. Given the high computational costs, as well as the costs of obtaining measured data for protein mutants, the systems and methods of the present disclosure, by converging faster than convention methods on mutants with desirable properties, computation time as well as wet lab resources.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first property could be termed a second property, and, similarly, a second property could be termed a first property, without departing from the scope of the present disclosure. The first property and the second property are both properties, but they are not the same property.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The present description, for purpose of explanation, is described with reference to specific implementations. However, the illustrative discussions herein are not intended to be exhaustive or to limit the implementations to the precise forms disclosed. Many modifications and variations are possible in view of the disclosed teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting.” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2: n≥5: n≥10; n≥25: n≥40: n≥50; n≥75; n≥100; n≥125; n≥150; n≥200; n≥225; n≥250; n≥350; n≥500; n≥600: n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

Furthermore, when a reference number is given an with “i^{th}” denotation, the reference number refers to a generic component, set, or embodiment. For instance, a property termed “property i” refers to the i^{th} property in a set of properties (e.g., a property 112-i in a set of properties 112).

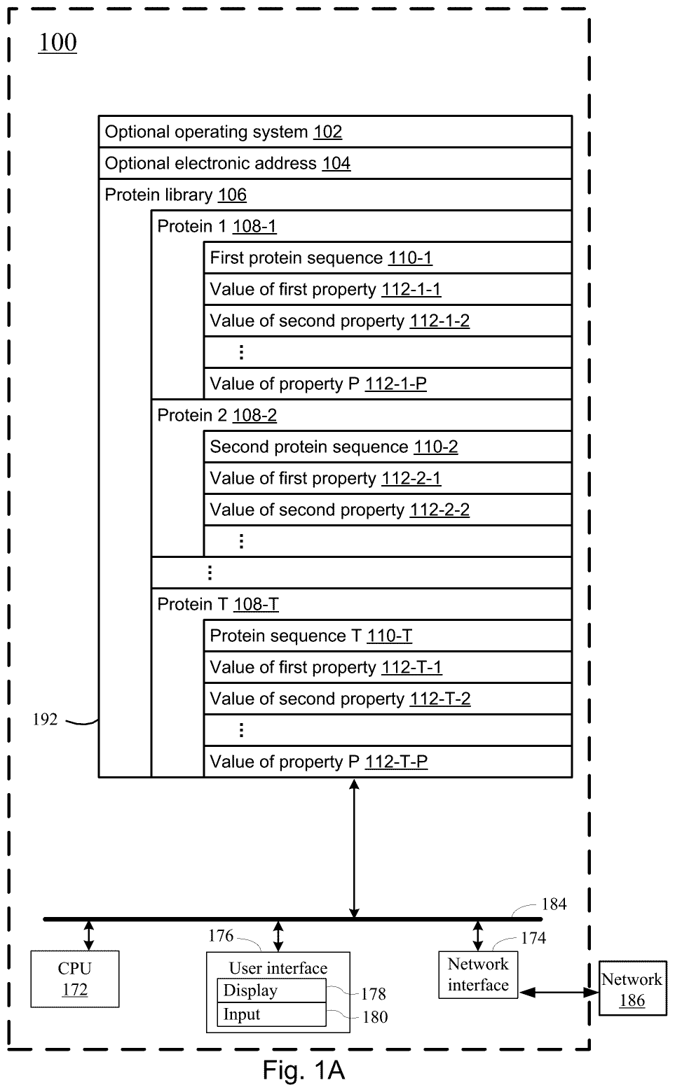

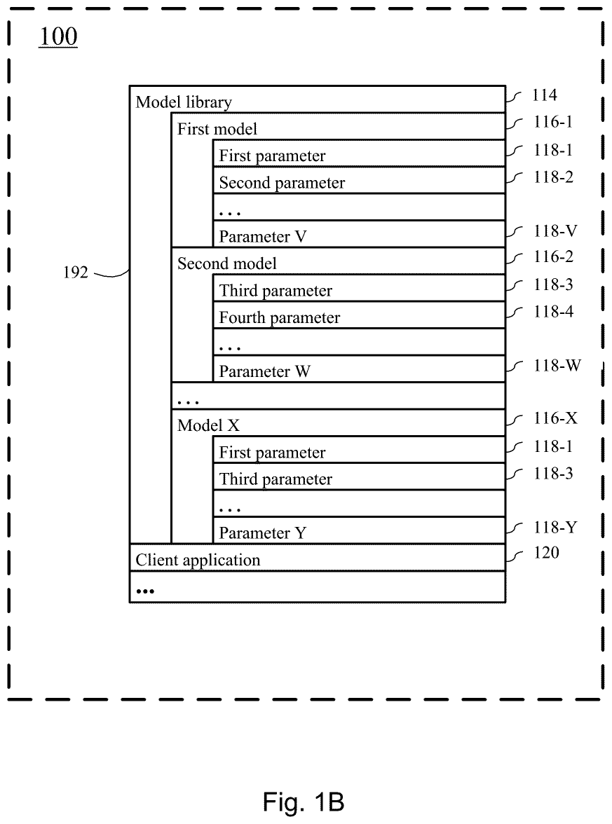

In the present disclosure, unless expressly stated otherwise, descriptions of devices and systems will include implementations of one or more computers. For instance, and for purposes of illustration in FIGS. 1A and 1B, a computer system 100 is represented as a single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 186 of FIG. 1A). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 186, the illustrated devices and systems may wirelessly transmit information between each other. As such, the exemplary topology shown in FIGS. 1A and 1B merely serves to describe the features of an embodiment of the present disclosure in a manner that will be readily understood to one of skill in the art.

FIGS. 1A and 1B collectively depicts a block diagram of a distributed computer system (e.g., computer system 100) according to some embodiments of the present disclosure. The computer system 100 at least facilitates identifying one or more combinatorial substitutions that affect one or more properties (e.g., a first property) a target protein.

In some embodiments, the communication network 186 optionally includes the Internet, one or more local area networks (LANs), one or more wide area networks (WANs), other types of networks, or a combination of such networks.

Examples of communication networks 186 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs) 172, a network or other communications interface 174, and memory 192.

In some embodiments, the computer system 100 includes a user interface 176. The user interface 176 typically includes a display 178 for presenting media, such as a result by a plurality of models (e.g., first model 116-1, second model 116-2, . . . , model X 116-X of FIG. 1B). In some embodiments, the display 178 is integrated within the computer systems (e.g., housed in the same chassis as the CPU 172 and memory 192). In some embodiments, the computer system 100 includes one or more input device(s) 180, which allow a subject to interact with the computer system 100. In some embodiments, input devices 180 include a keyboard, a mouse, and/or other input mechanisms. Alternatively, or in addition, in some embodiments, the display 178 includes a touch-sensitive surface (e.g., where display 178 is a touch-sensitive display or computer system 100 includes a touch pad).

In some embodiments, the computer system 100 presents media to a user through the display 178. Examples of media presented by the display 178 include one or more images, a video, audio (e.g., waveforms of an audio sample), or a combination thereof. In typical embodiments, the one or more images, the video, the audio, or the combination thereof is presented by the display 178 through a client application 120. In some embodiments, the audio is presented through an external device (e.g., speakers, headphones, input/output (I/O) subsystem, etc.) that receives audio information from the computer system 100 and presents audio data based on this audio information. In some embodiments, the user interface 176 also includes an audio output device, such as speakers or an audio output for connecting with speakers, earphones, or headphones.

Memory 192 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 192 may optionally include one or more storage devices remotely located from the CPU(s) 172. Memory 192, or alternatively the non-volatile memory device(s) within memory 192, includes a non-transitory computer readable storage medium. Access to memory 192 by other components of the computer system 100, such as the CPU(s) 172, is, optionally, controlled by a controller. In some embodiments, memory 192 can include mass storage that is remotely located with respect to the CPU(s) 172. In other words, some data stored in memory 192 may in fact be hosted on devices that are external to the computer system 100, but that can be electronically accessed by the computer system 100 over an Internet, intranet, or other form of network 186 or electronic cable using communication interface 184.

In some embodiments, the memory 192 of the computer system 100 for identifying one or more combinatorial substitutions that affect one or more properties (e.g., a first property) of a target protein stores:
optionally, an operating system 102 (e.g., ANDROID, IOS, DARWIN, RTXC, LINUX, UNIX, OS X, WINDOWS, or an embedded operating system such as VxWorks) that includes procedures for handling various basic system services:
optionally, an electronic address 104 associated with the computer system 100 that identifies the computer system 100 (e.g., within the communication network 186):
a protein library 106 that stores a record of a plurality of proteins (e.g., protein 108-1, protein 108-2, . . . , protein 108-T of FIG. 1A), each protein 108 defined by a protein sequences (e.g., first protein sequence 110-1, second protein sequence 110-2, . . . , protein sequence 110-T 1 of FIG. 1A), whereby each protein is characterized by a plurality of properties (e.g., value of first property 112-1-1, value of second property 112-2-1, . . . , value of property P 112-1-P of protein 108-1 of FIG. 1A) that is utilized by one or more models 116 for identifying the one or more combinatorial substitutions that affect a first (target protein:
a model library 114 that retains a plurality of models (e.g., first model 116-1, second model 116-2, . . . , model X 116-X of FIG. 1B), each respective model 116 for providing, at least in part, an identity of the one or more combinatorial substitutions that affect the first property of the target protein based on one or more parameters of a corresponding model 116; and
a client application 120 for presenting information (e.g., media) using a display 178 of the computer system 100.

As indicated above, an optional electronic address 104 is associated with the computer system 100. The optional electronic address 204 is utilized to at least uniquely identify the computer system 100 from other devices and components of the distributed system 100, such as other devices having access to the communications network 186. For instance, in some embodiments, the electronic address 104 is utilized to receive a request from a remote device to identify one or more combinatorial substitutions that affect a property of a target protein.

The protein library 106 stores a record of a plurality of proteins 108. In some embodiments, the protein library 107 stores greater than 100 proteins 108, greater than 500 proteins 108, greater than 1,000 proteins 108, greater than 10,000 proteins 108, greater than 100,000 proteins 108, greater than 1 million proteins 108, or greater than a billion proteins 108. By “protein” herein is meant at least two amino acids linked together by a peptide bond. Accordingly, each respective protein 108 is defined by a sequence of amino acids (e.g., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine), which is linked by bonds. As such, the sequence of amino acids is a linear sequence of a positions having an initial N-terminal position, one or more intermediate positions, and a C-terminal position. Accordingly, in some embodiments, each respective position is associated with a corresponding residue of the respective protein 108. As a non-limiting example, consider a target protein 108 that includes a sequence of approximately 500 amino acids (e.g., N-terminal position of a first amino acid, second position of a second amino acid, . . . , C-terminal position 500 of a five-hundredth amino acid of a 108 of FIG. 1A). Accordingly, based on the limited universe of twenty naturally occurring amino acids, there are 20500 possible amino acid combinations for the sequence of the protein 100. Moreover, while in some embodiments, the proteins under study are limited to the twenty naturally occurring amino acids, in some embodiments the proteins are not so limited. In some embodiments, the disclosed proteins can have unnatural amino acids such as 2-aminoadipic acid, 3-aminoadipic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, 6-N-methyllysine, N-methylvaline, norvaline, norleucine, and/or ornithine, to name some non-limiting examples.

In some embodiments, the respective protein 108 of the protein library 106 is based on a wild type protein 108. As the wild type, the respective protein 108 characterizes a gene or phenotype that is found in a natural, non-mutated (e.g., unchanged) form. In some such embodiments, the wild type protein 108 acts as a reference sequence of a plurality of positions within the a target protein 108, where each such position represents a particular residue found in the naturally occurring protein. In some embodiments, a respective position is variable, such that an amino acid is alterable by the systems and methods of the present disclosure. In some embodiments, the respective position is fixed, such that the amino acid is fixed by the systems and methods of the present disclosure. For instance amino acid positions that are not observed to change in nature, for instance across homologs of the target protein, are fixed by the systems and method of the present disclosure. In other words, single point mutations at these fixed positions are not explored by the systems and method of the present disclosure. Any number of reasons may cause a position to be fixed. For instance, the amino acid (residue) at the fixed position may be part of a key enzymatic reaction of the target protein, essential to the stability of the tertiary structure of the target protein, and so forth. On the other hand, positions in the target protein that are observed to change across homologs of the protein are the subject of mutational search using the systems and methods of the present disclosure.

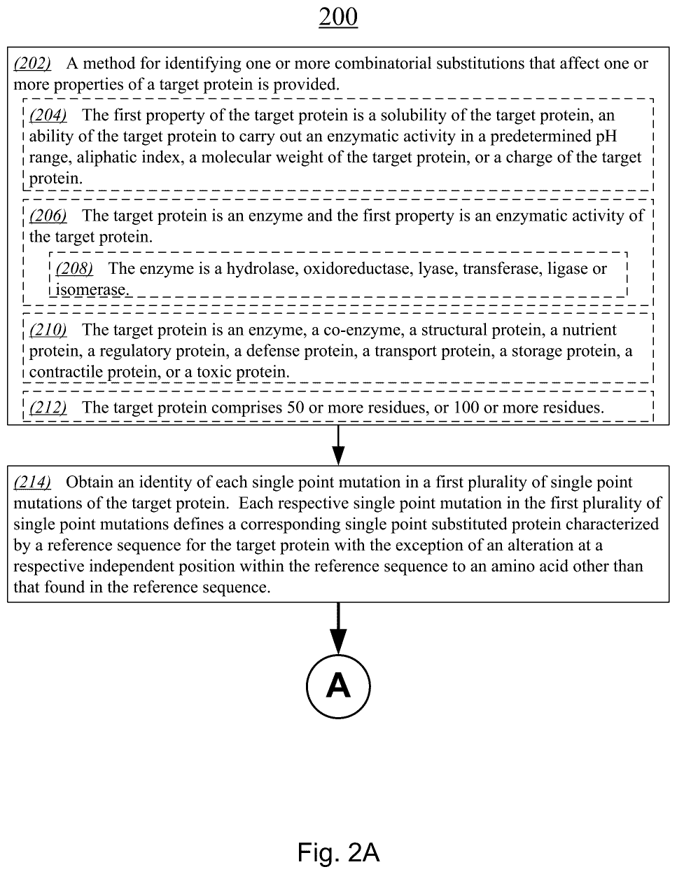

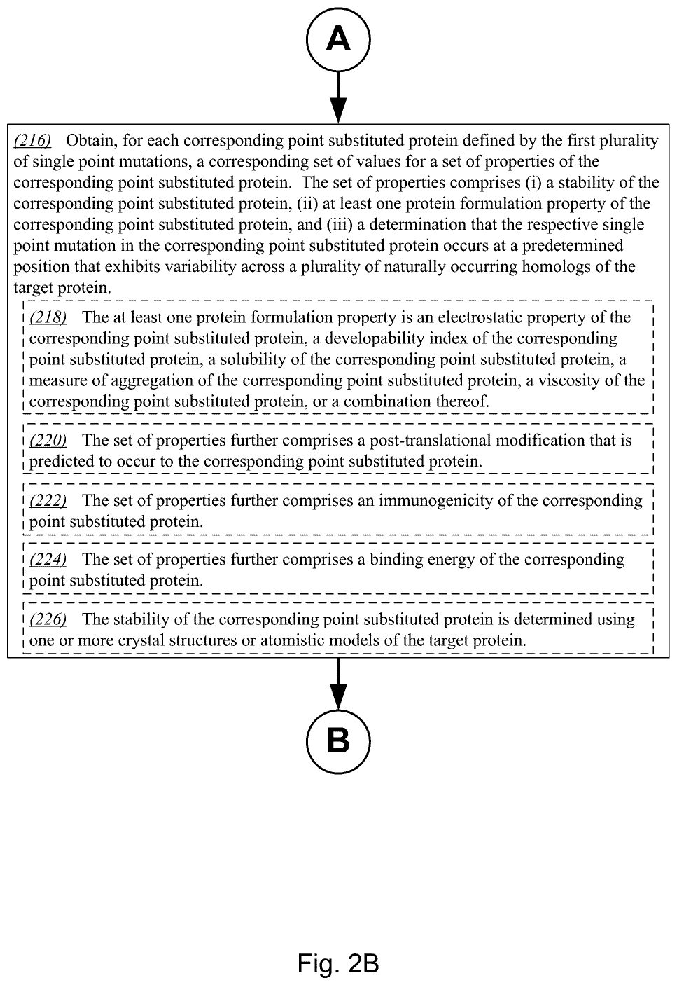

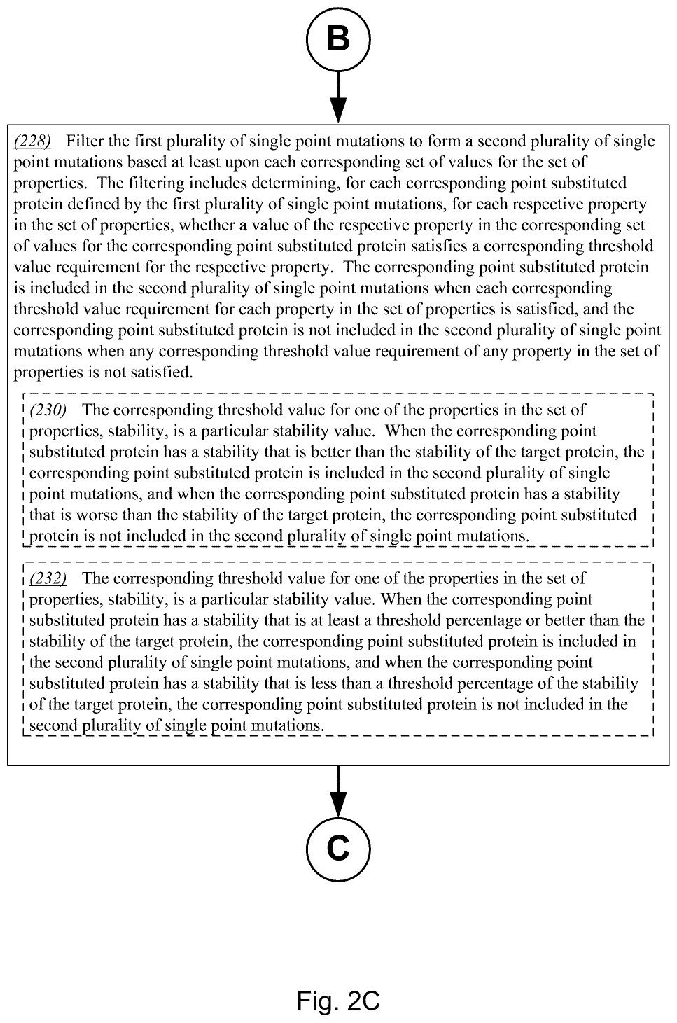

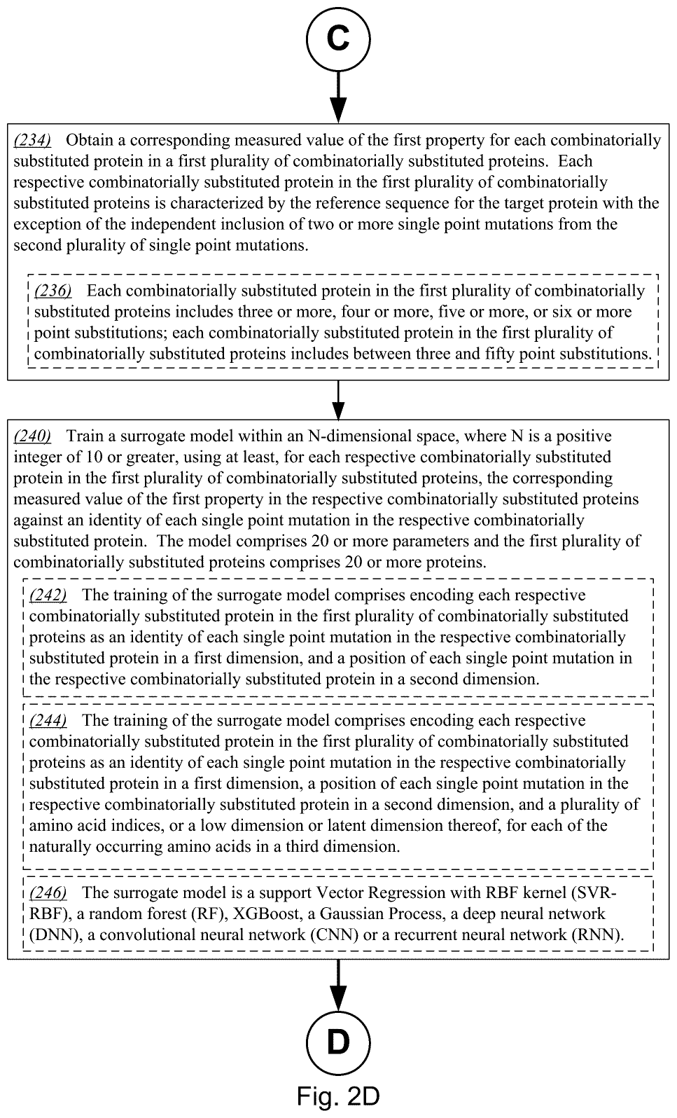

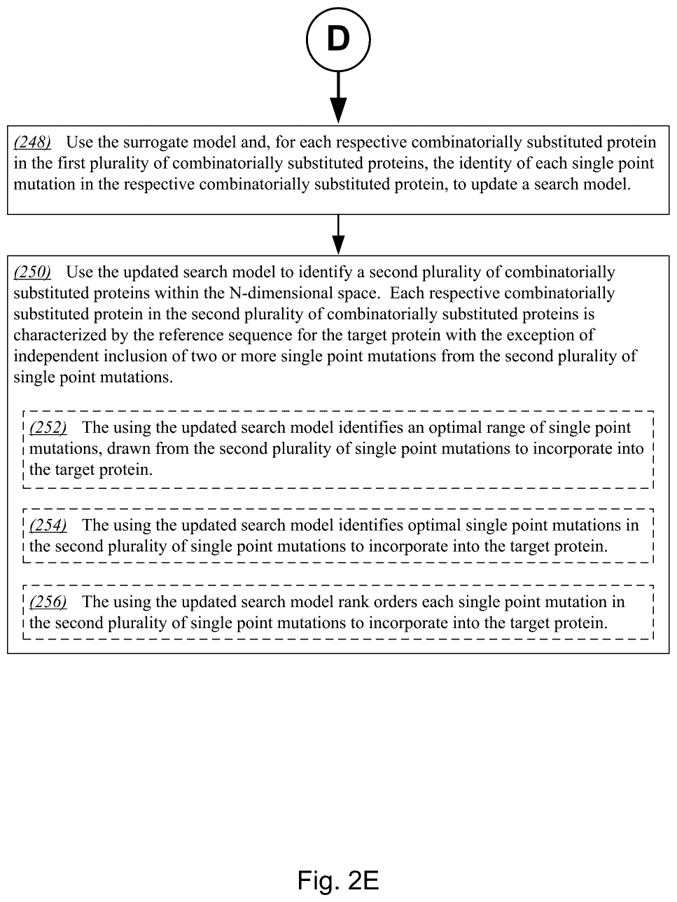

Each property 112 of a respective protein 108 in the protein library 106 is a physical or chemical behavior of the protein 108. As a non-limiting example, in some embodiments, a respective protein 108 in the protein library is associated with one more electrostatic properties (e.g., an isoelectric point property, a pH of maximal stability property, a net charge property, a dipole moment property, etc.), an aggregation property, a solubility property, a developability index property (e.g., a tendency to aggregate property), one or more viscosity scores, an ionic strength, an opalescence of the protein, a immunogenicity of the protein 108 (e.g., block 222 of FIG. 2B), or a combination thereof. For instance, in some embodiments, a respective property 112 is an opalescence, a viscosity, a protein aggregation, a protein immunogenicity, or the like. In some embodiments, a respective property 112 is a mutation energy (e.g., binding). This mutation energy allows the computer system 100 to ensure a substrate of the respective protein 108 still binds the enzyme active site correctly after mutagenesis in order to make active enzyme catalyst.

Referring to FIG. 1B, the computer system includes a model library 114 that stores a plurality of models 116 (e.g., classifiers, regressors, clustering, etc.). In some embodiments, the model library 114 stores two more models 116 (e.g., a first surrogate model 116-1 and a second search model 116), three or more models (e.g., the first surrogate model 116-1, a second first state search model 116, a third second state search model 116), four or more models, ten or more models, 50 or more models, or 100 or more models. In some embodiments, a model 116 in the plurality of models 116 is implemented as an artificial intelligence engine. For instance, in some embodiments, the model 116 includes one or more gradient boosting models 116, one or more random forest models 116, one or more neural network (NN) models 116, one or more regression models, one or more Naïve Bayes models 116, one or more machine learning algorithms (MLA) 116, or a combination thereof. In some embodiments, an MLA or a NN is trained from a training data set that includes one or more features identified from a data set. MLAs include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated a priori), such as means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as minimum cut, harmonic function, manifold regularization, etc.), heuristic approaches, or support vector machines.

Neural network models 116 include conditional random fields models 116, convolutional neural network (CNN) models 116, attention based neural network models 116, deep learning models 116, long short term memory network model 116, or other neural models 116.

While MLA and neural networks identify distinct approaches to machine learning, the terms may be used interchangeably herein. Thus, a reference to MLA may include a corresponding NN or a reference to NN may include a corresponding MLA unless explicitly stated otherwise. In some embodiments, the training of a respective model includes providing one or more optimized data sets, labeling these features as they occur (e.g., in user profile 16 records), and training the MLA to predict or classify based on new inputs. Artificial NNs are efficient computing models which have shown their strengths in solving hard problems in artificial intelligence. For instance, artificial NNs have also been shown to be universal approximators, that is, they can represent a wide variety of functions when given appropriate parameters.

Accordingly, in some embodiments, a first model 116-1 in the plurality of models 116 is a surrogate model (e.g., block 240 through block 246 of FIG. 2D and block 248 of FIG. 2E or a combination thereof) and a second model 116-2 in the plurality of models 116 is a search model (e.g., block 250 through block 256 or a combination thereof of FIG. 2E). However, the present disclosure is not limited thereto.

One of skill in the art will readily appreciate other models 116 that are applicable to the systems and methods of the present disclosure. In some embodiments, the systems and methods of the present disclosure utilize more than one model 116 to provide an evaluation (e.g., arrive at an evaluation given one or more inputs), such as an identity of one or more combinatorial substitutions that affect a first property of a target protein 108 (e.g., first protein 108-1) with an increased accuracy. For instance, in some embodiments, each respective model 116 arrives at a corresponding evaluation when provided a respective data set. Accordingly, in some embodiments, each respective model 116 independently arrives at a result and then the result of each respective model 116 is collectively verified through a comparison or amalgamation of the models 116. From this, a cumulative result is provided by the models 116. However, the present disclosure is not limited thereto.

In some embodiments, a respective model 116 is tasked with performing a corresponding activity. As a non-limiting example, in some embodiments, the task performed by the respective model 116 includes, but is not limited to, identifying one or more combinatory substitutions (e.g., block 202 of FIG. 2A), identifying a first property 112-1 (e.g., block 202 of FIG. 2A), identifying a target protein 108 (e.g., block 202 of FIG. 2A), obtaining an identification of each single point mutation in a first plurality of single point mutations (e.g., block 214 of FIG. 2A), obtaining a corresponding set of values for a set of properties 112 of a corresponding point substituted protein defined by the first plurality of single point mutations (e.g., block 216 of FIG. 2B), filtering the first plurality of single point mutations to form a second plurality of single point mutations (e.g., block 228 of FIG. 2C), determining whether a value of a respective property 112 in the corresponding set of values for the corresponding point substituted protein satisfies a corresponding threshold value requirement for the respective property (e.g., block 228 of FIG. 2C), obtaining a corresponding measured value of the first property for each combinatorially substituted protein in a first plurality of combinatorially substituted protein (e.g., block 238 of FIG. 2D), training within an N-dimensional space (e.g., block 240 of FIG. 2D), determining an identify of each single point mutation in a first plurality of combinatorially substituted proteins (e.g., block 248 of FIG. 2E), updating a search model (e.g., block 248 of FIG. 2E), identifying a second plurality of combinatorially substituted proteins within the N-dimensional space (e.g., block 250 of FIG. 2E), or any combination thereof.

In some embodiments, the first plurality of combinatorially substituted proteins in D) has mutation rates configured to allow learning of comprehensive interactions between different mutations. For example, the learning of comprehensive interactions comprises learning of both linear (PF_{AB}=PF_{A}*PF_{B}) and non-linear interactions between mutations (PF_{AB}< or >PF_{A}*PF_{B}, also referred as epistatic interactions in the Examples); learning of interactions between mutations occurring at any positions of the protein across the 1-dimensional sequence space and 3-dimensional structure space; and/or learning of rich interactions between 2-N, where N is the number of mutations per mutant.

In some embodiments, each respective model 116 of the present disclosure makes use of 10 or more parameters, 100 or more parameters, 1000 or more parameters, 10,000 or more parameters, or 100,000 or more parameters. In some embodiments, each respective model of the present disclosure cannot be mentally performed.

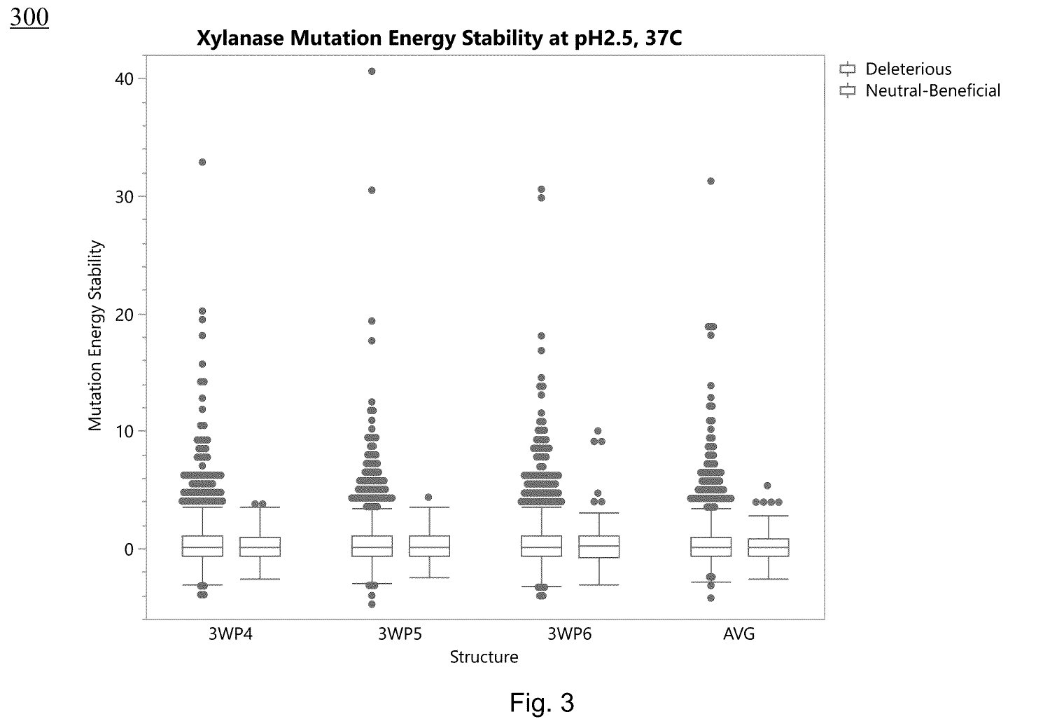

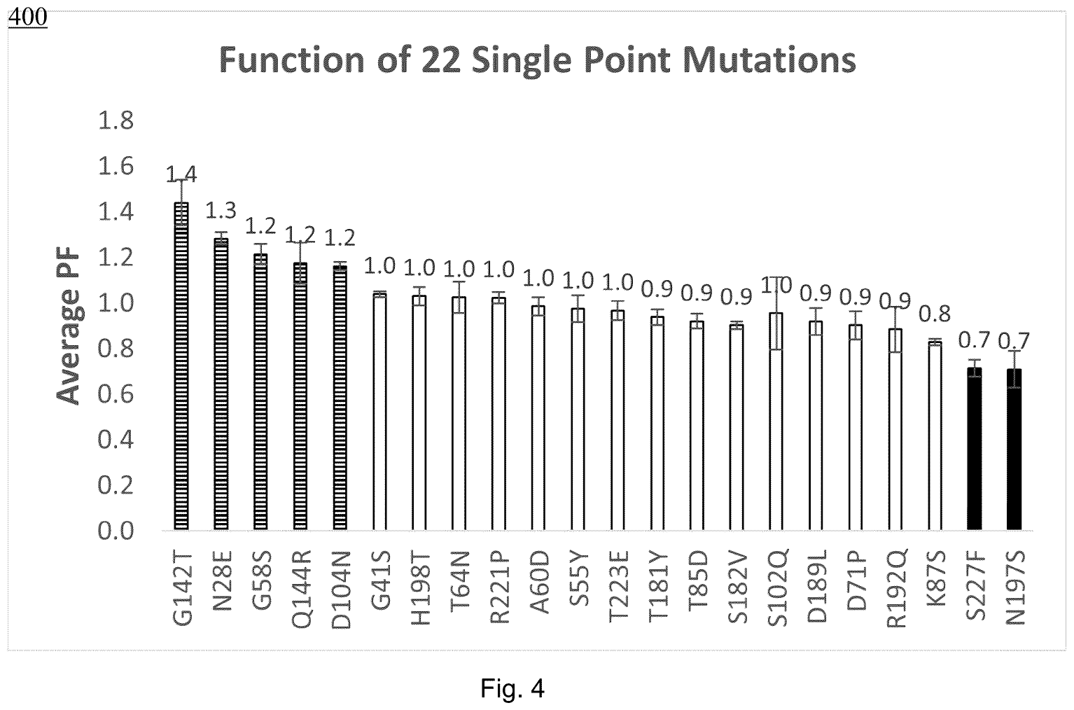

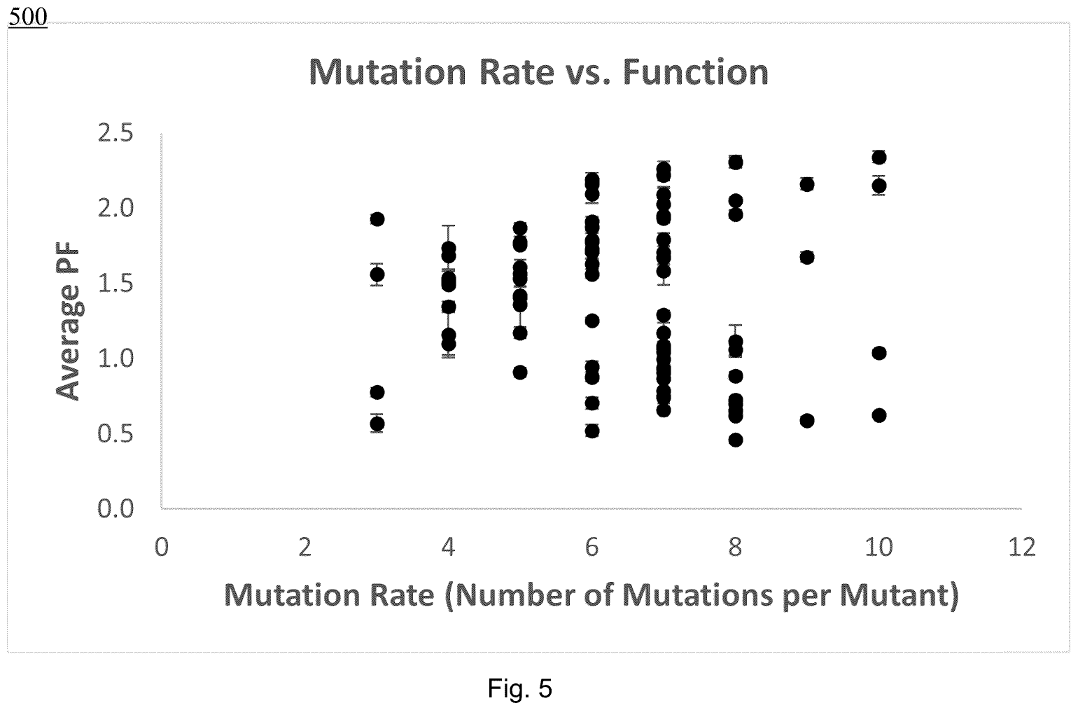

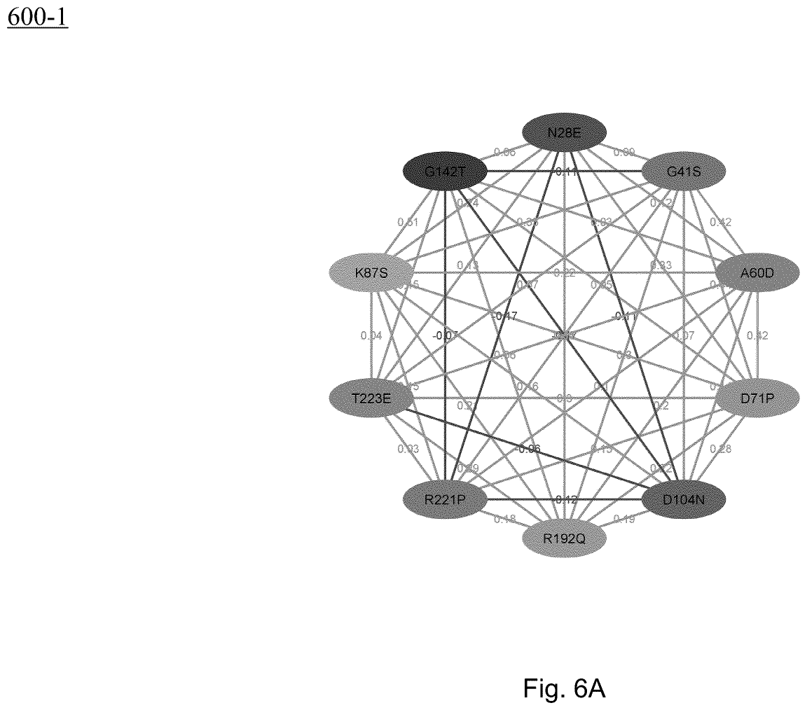

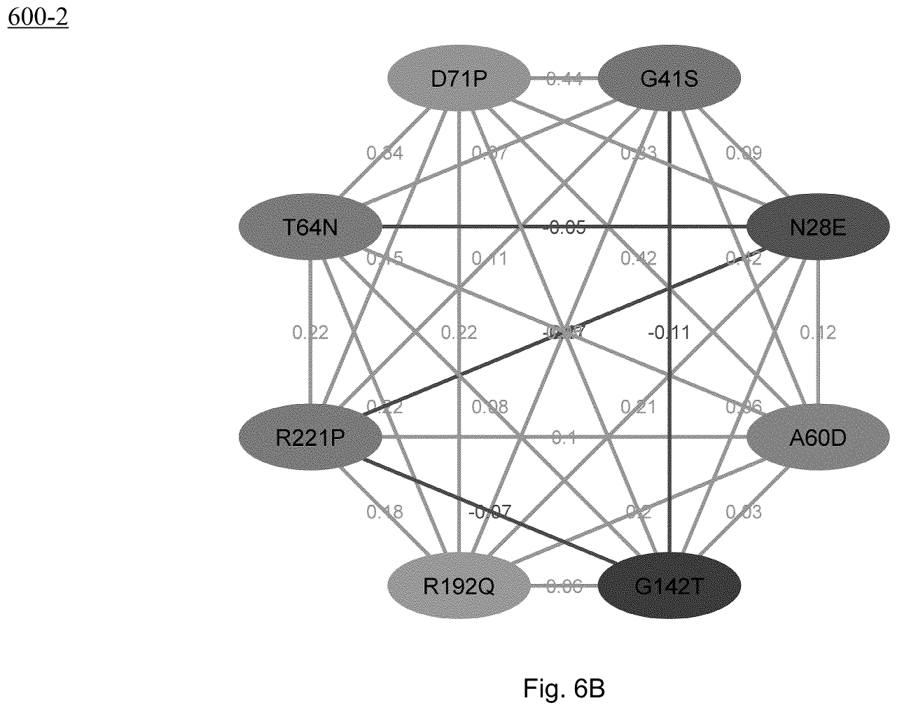

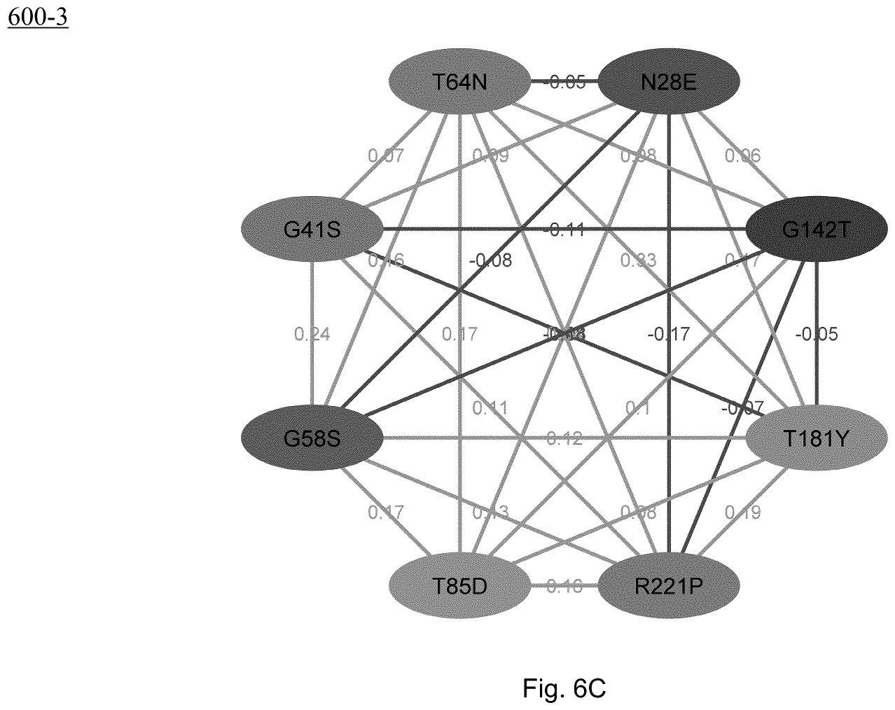

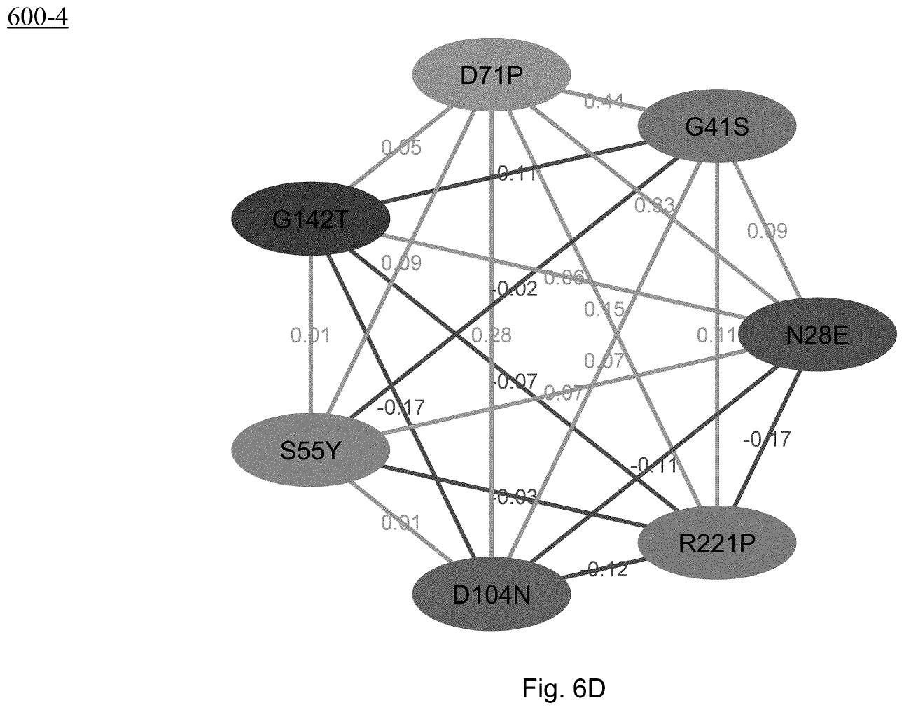

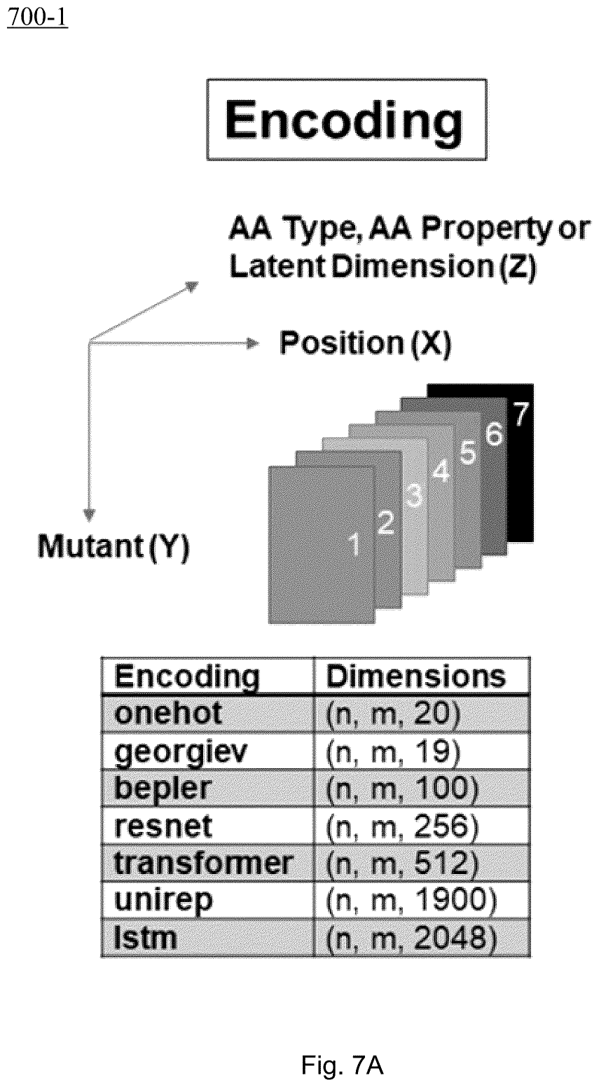

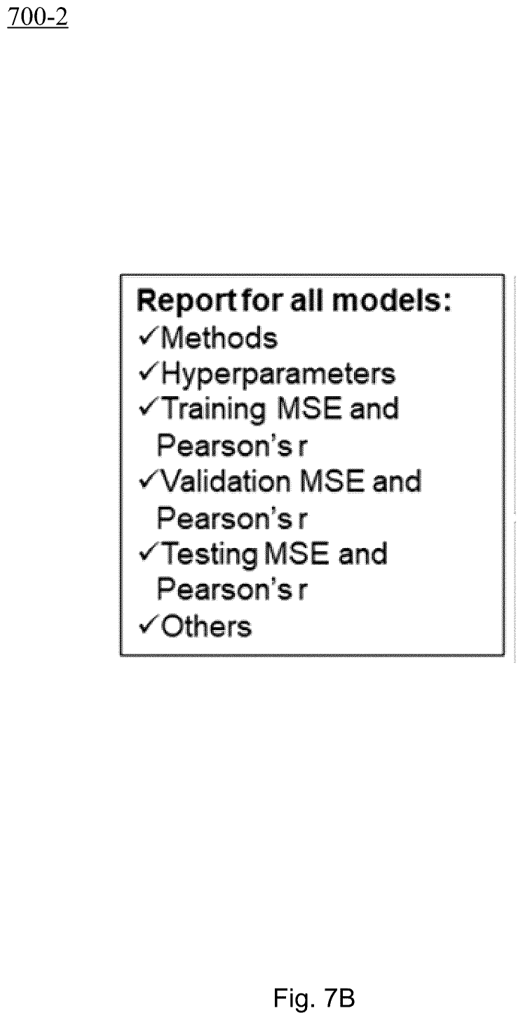

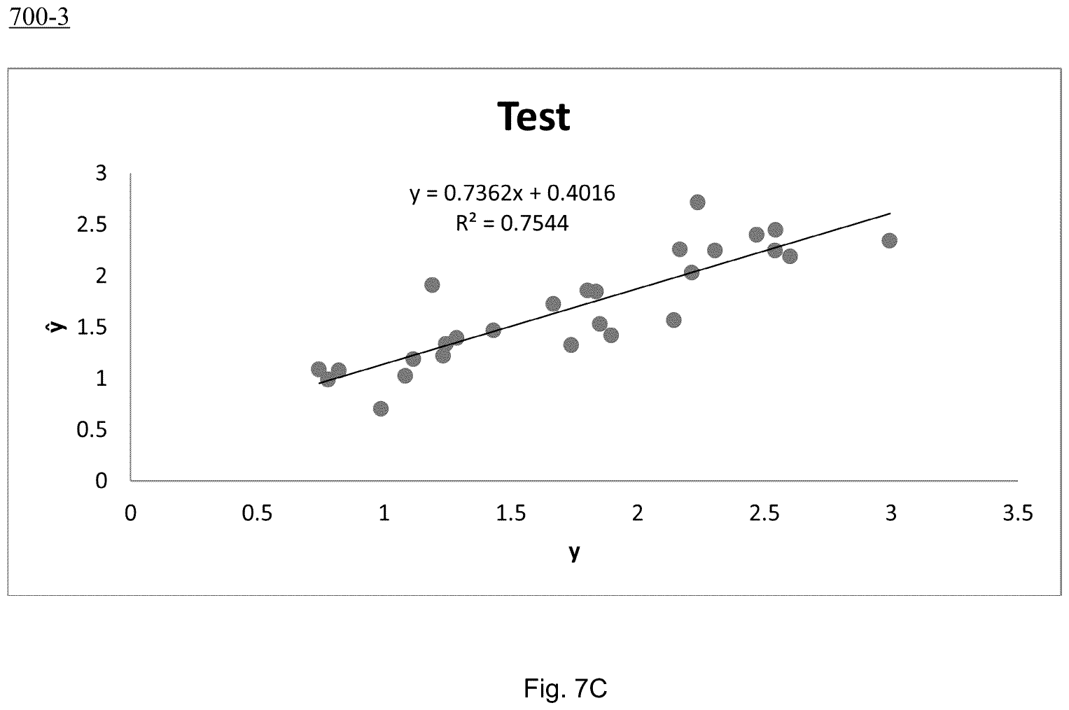

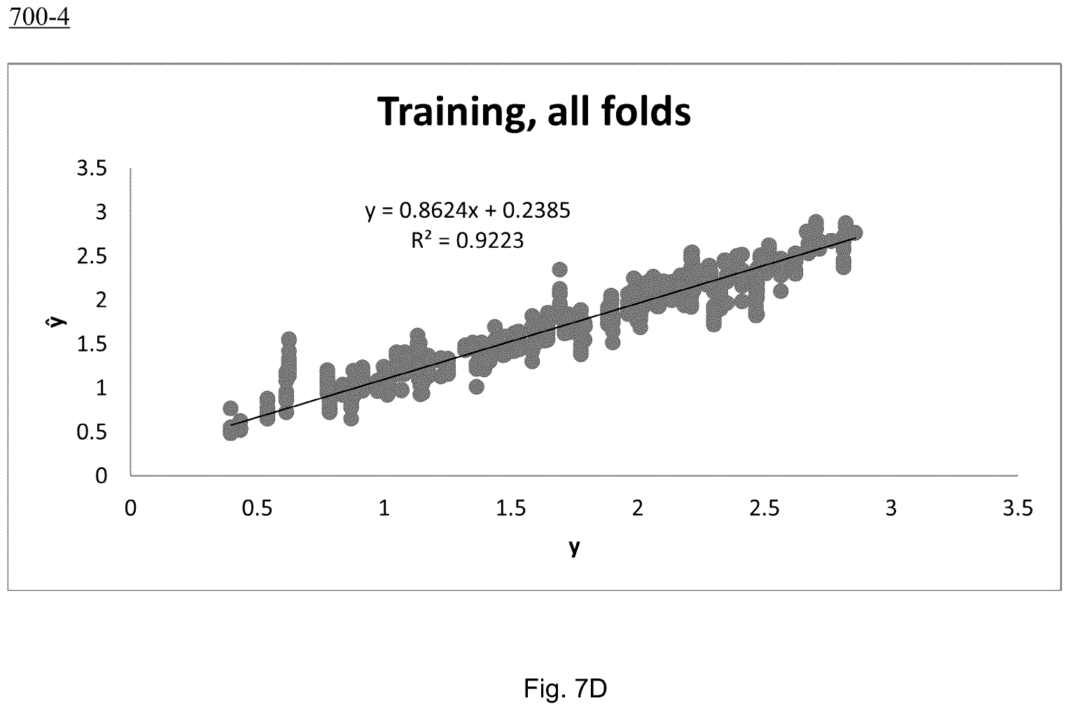

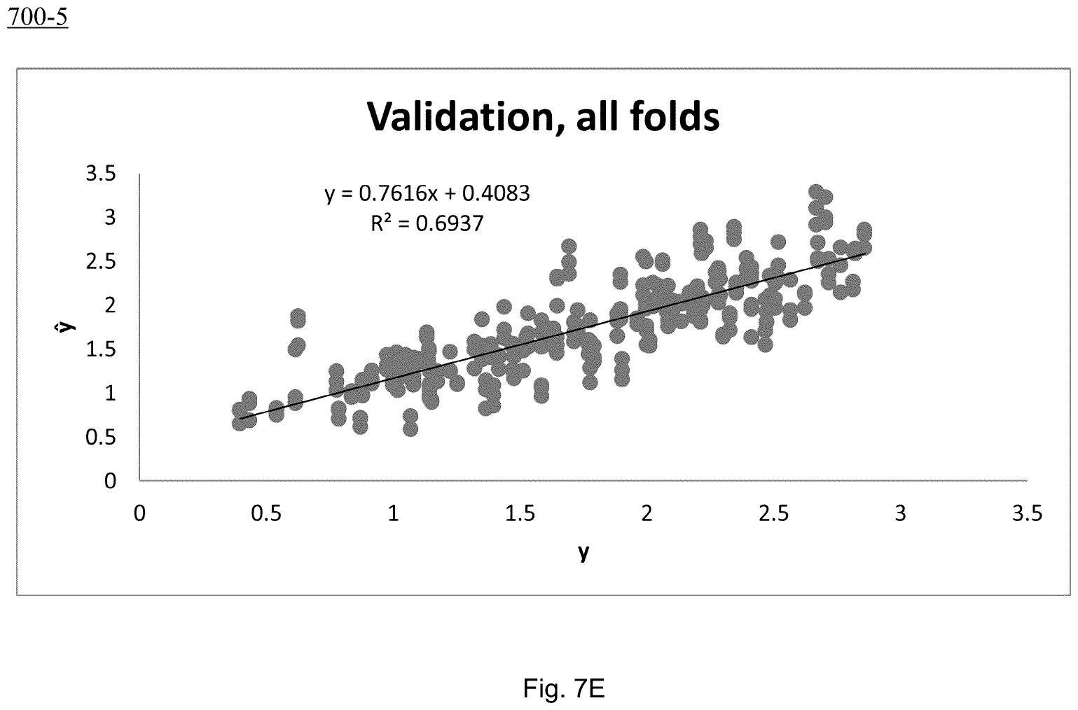

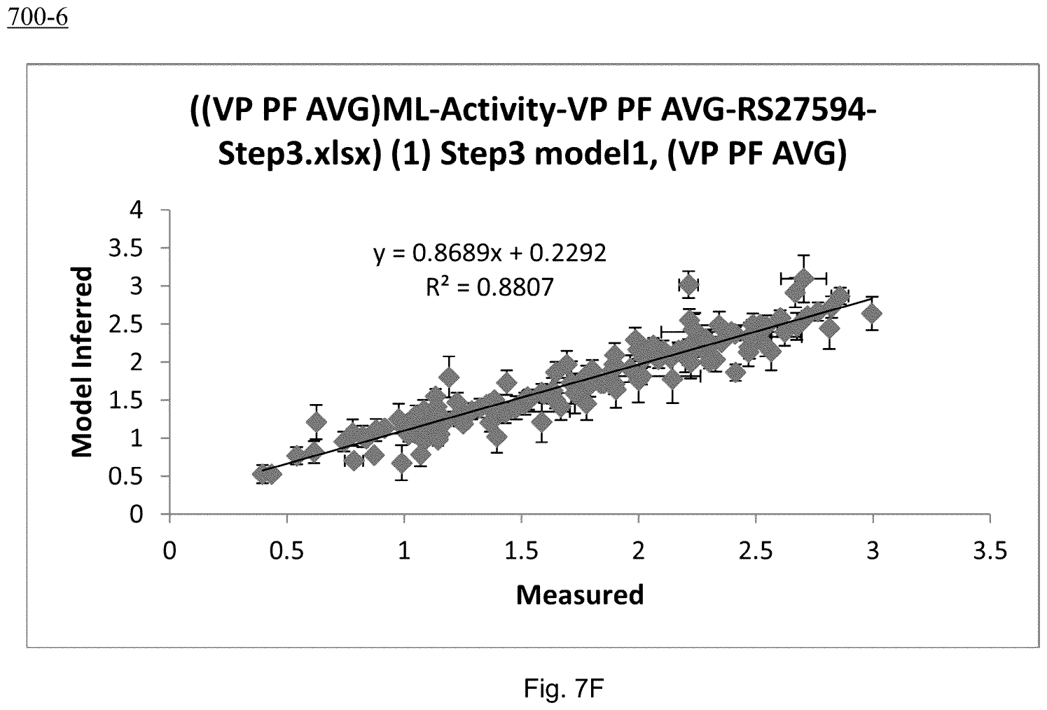

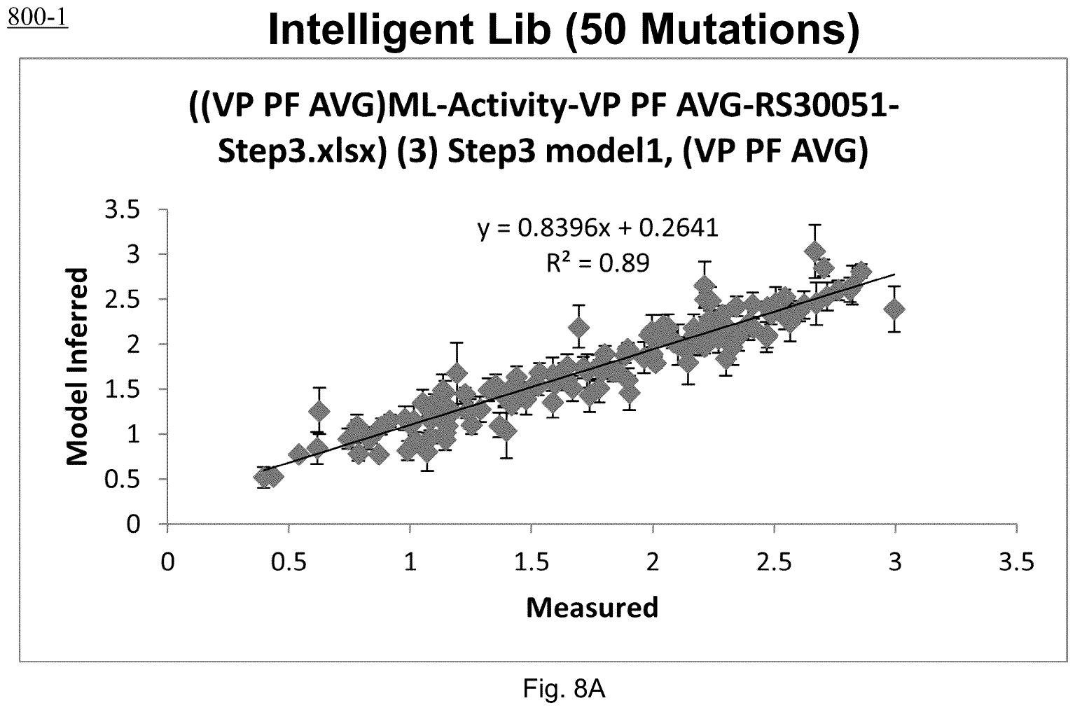

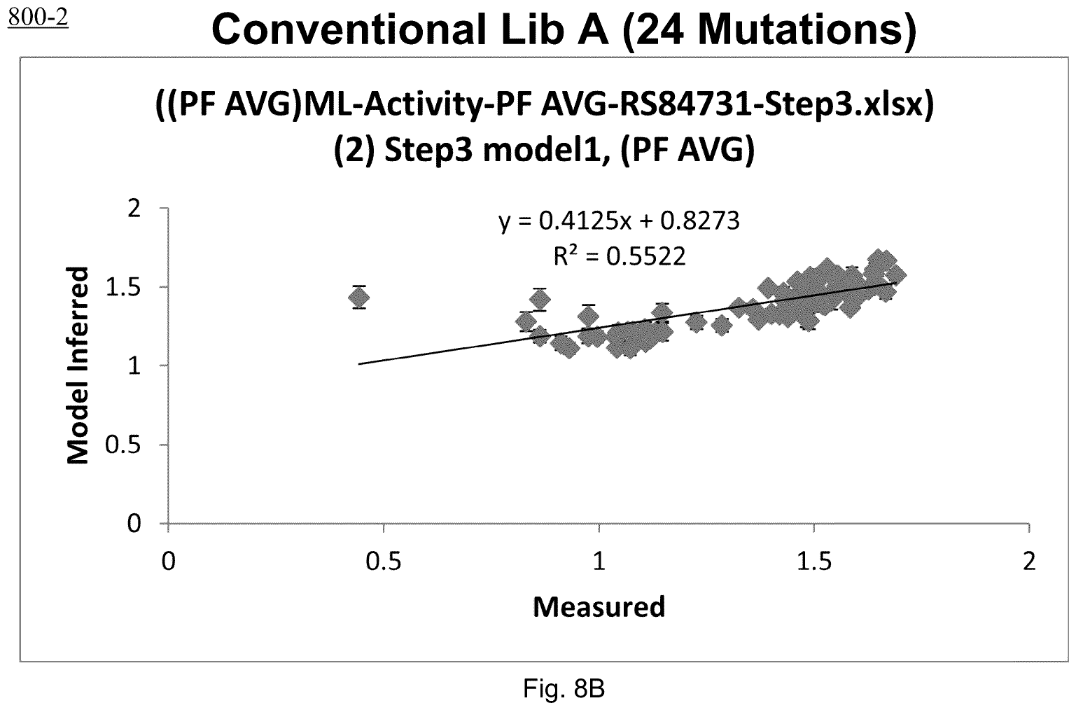

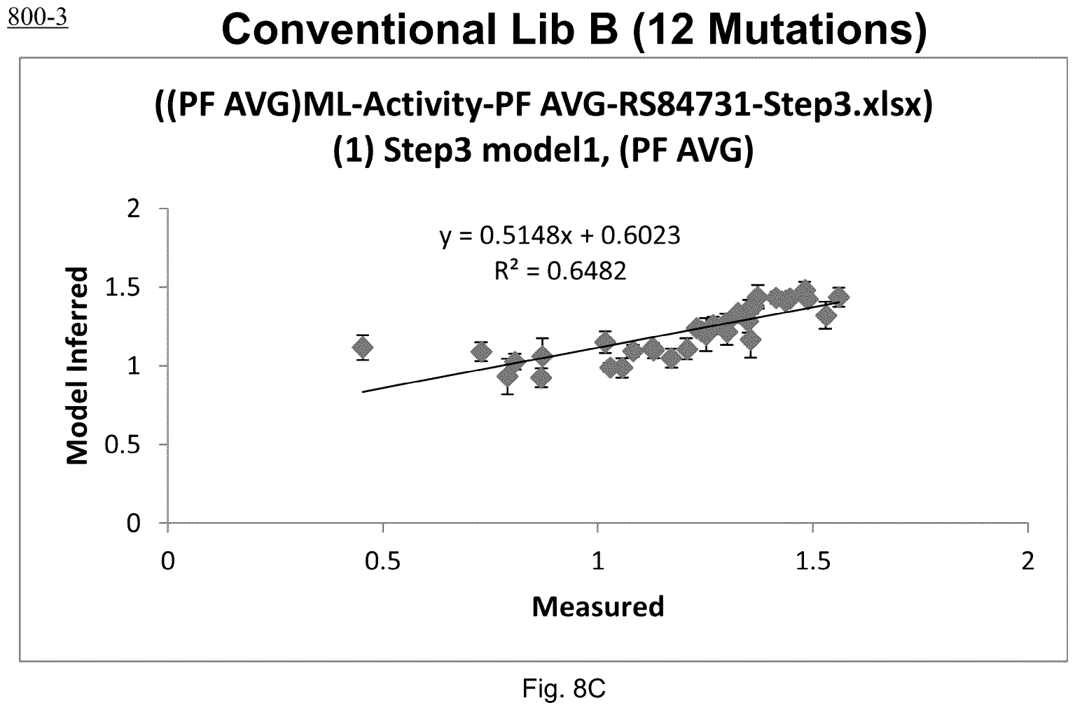

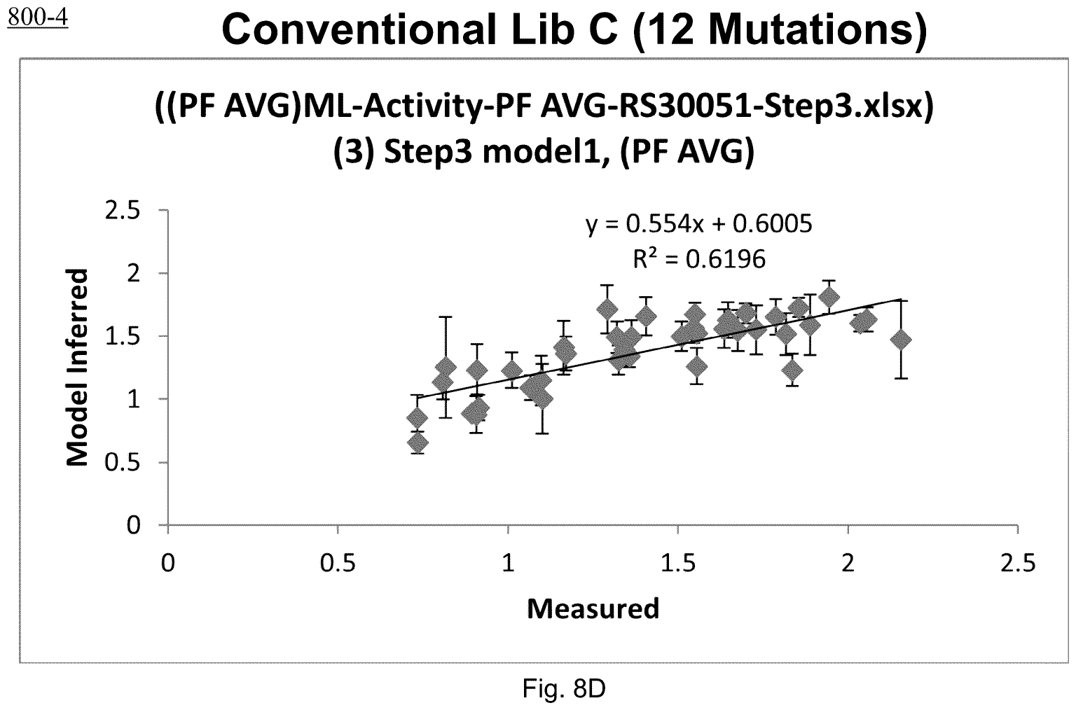

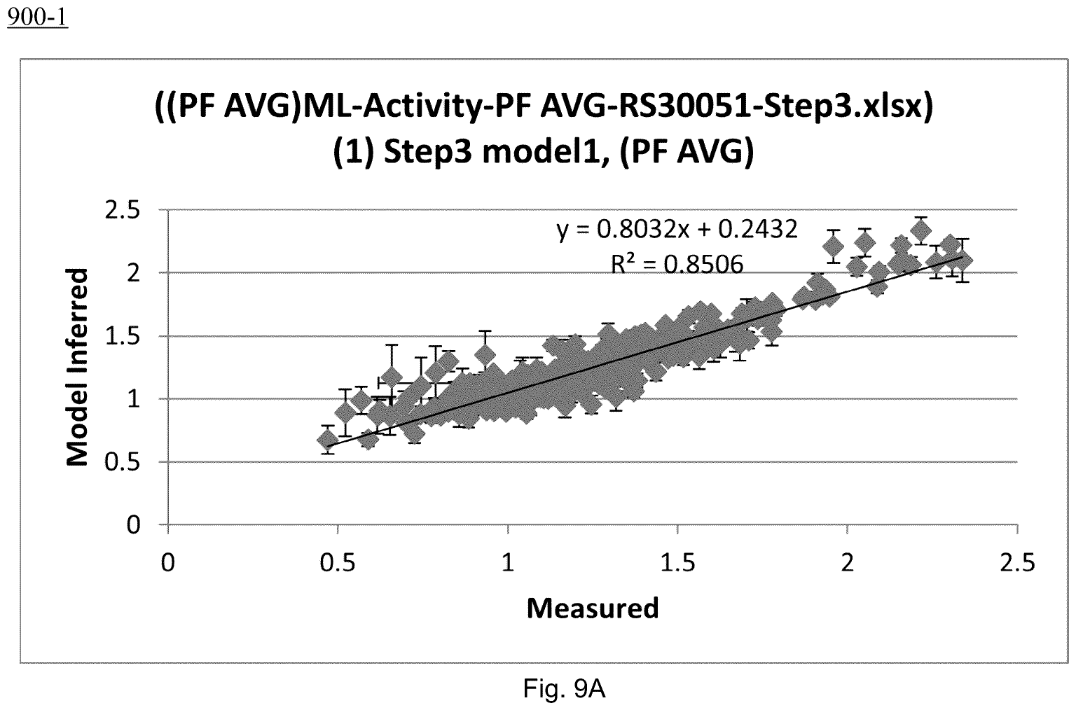

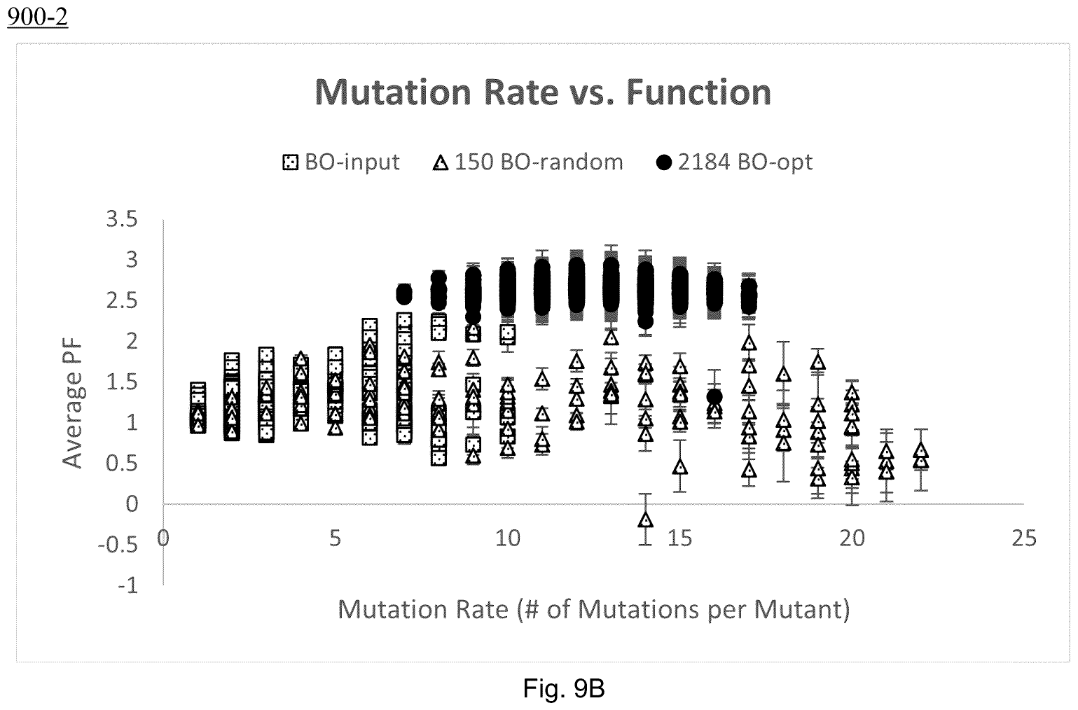

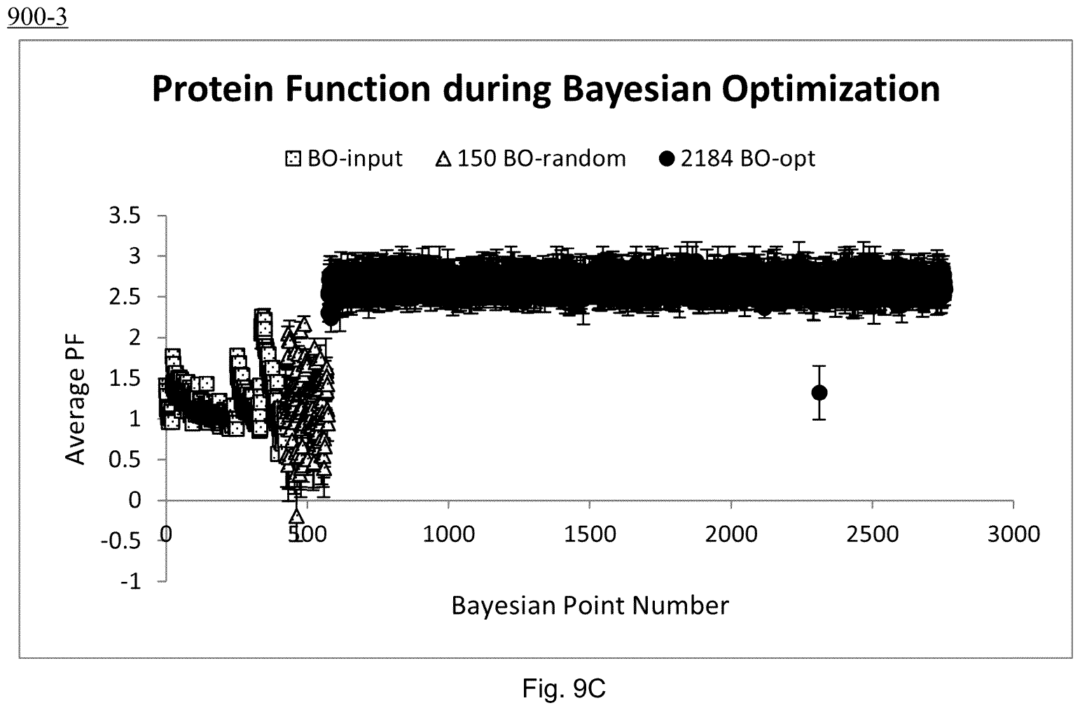

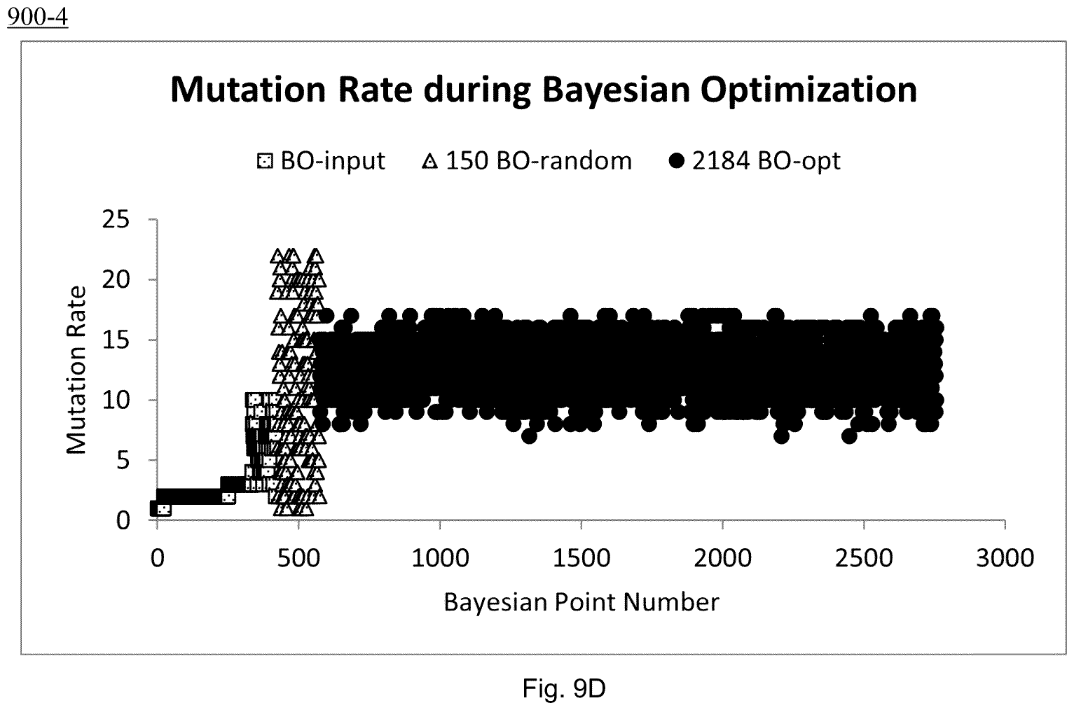

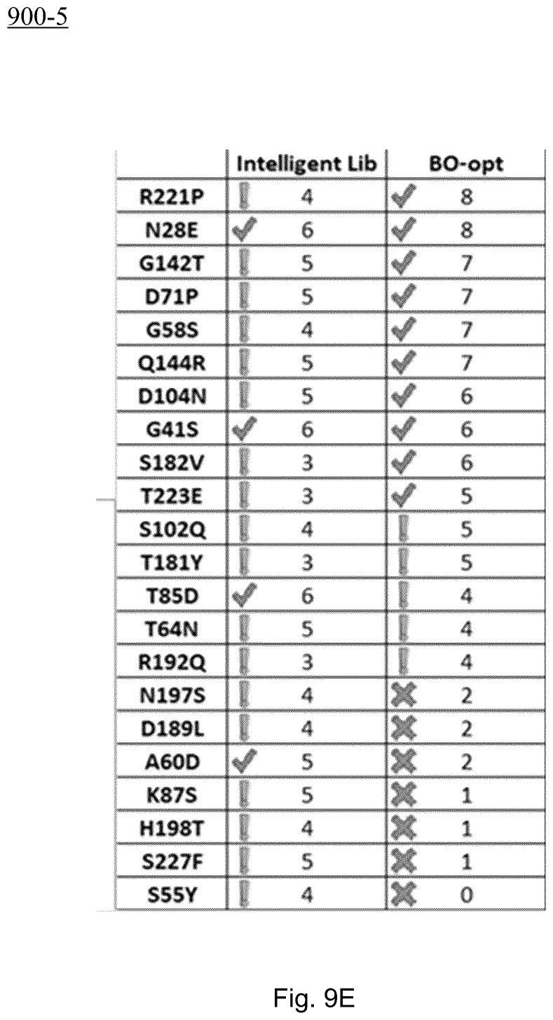

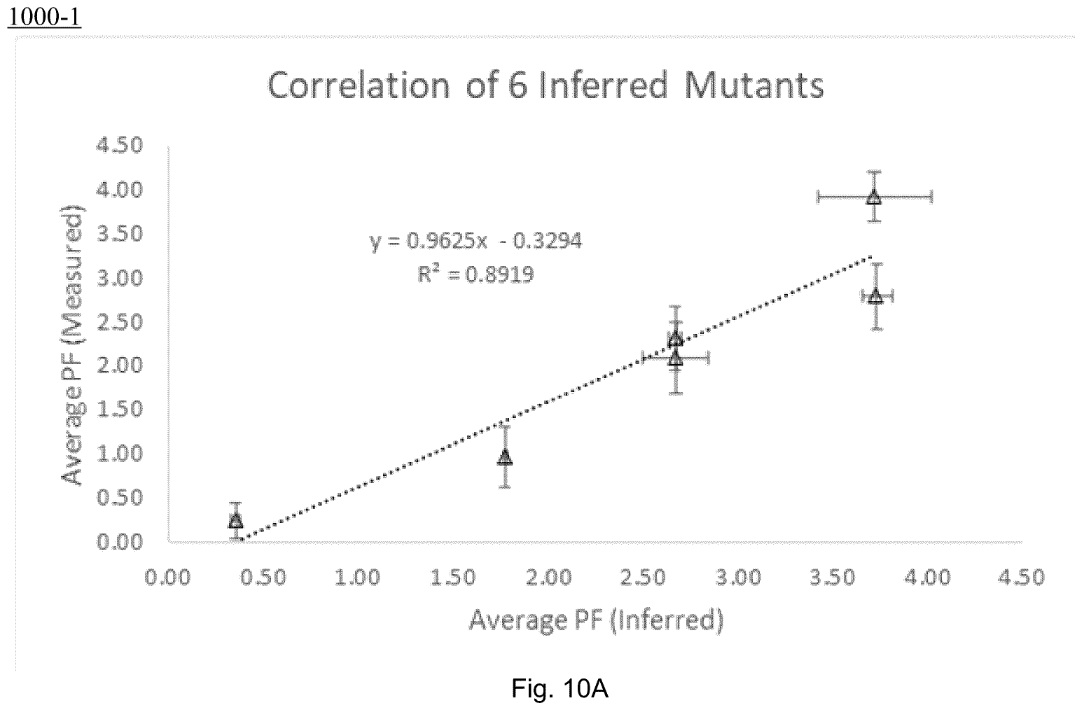

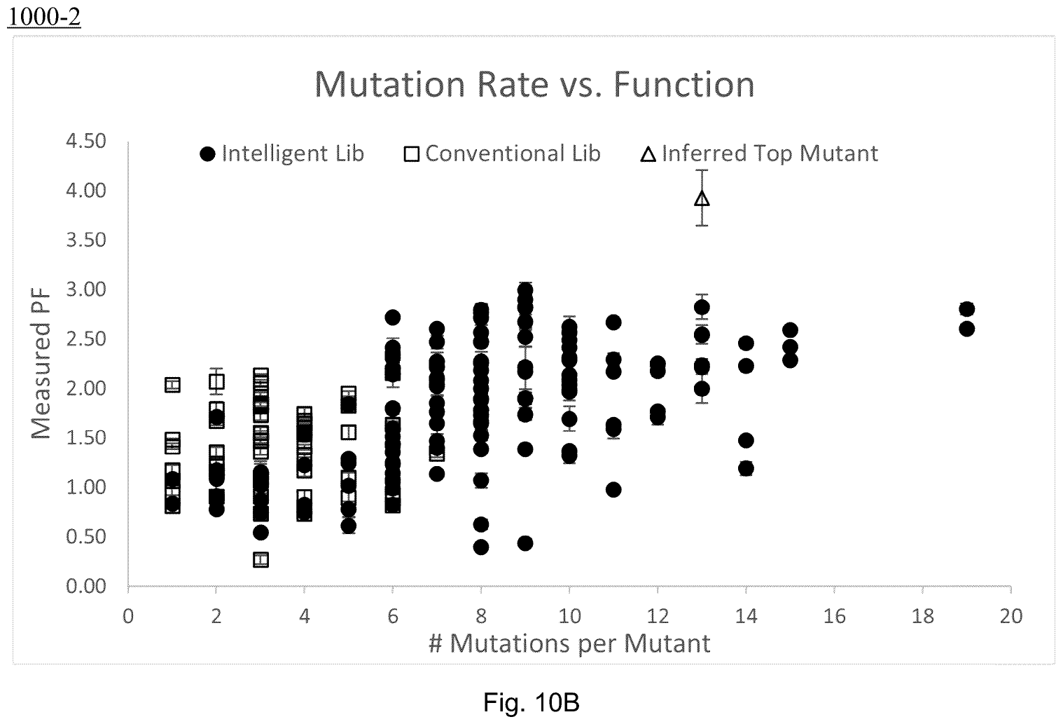

In some embodiments, a client application 120 is a group of instructions that, when executed by the processor 174, generates content for presentation to the user (e.g., user interface 300 of FIG. 3, user interface 400 of FIG. 4, user interface 500 of FIG. 5, user interface 600-1 of FIG. 6A, user interface 600-2 of FIG. 6B, user interface 600-3 of FIG. 6C, user interface 600-4 of FIG. 6D, user interface 700-1 of FIG. 7A, user interface 700-2 of FIG. 7B, user interface 700-3 of FIG. 7C, user interface 700-4 of FIG. 7D, user interface 700-5 of FIG. 7E, user interface 800-1 of FIG. 8A, user interface 800-2 of FIG. 8B, user interface 800-3 of FIG. 8C, user interface 800-4 of FIG. 8D, user interface 900-1 of FIG. 9A, user interface 900-2 of FIG. 9B, user interface 900-3 of FIG. 9C, user interface 900-4 of FIG. 9D, user interface 1000-1 of FIG. 10A, user interface 1000-2 of FIG. 10B, or a combination thereof), such as a result provided by one or more models 116. In some embodiments, the client application 120 generates content in response to one or more inputs received from the user through the computer system 100, such as the inputs 180 of the computer system 100.

Each of the above identified modules and applications correspond to a set of executable instructions for performing one or more functions described above and the methods described in the present disclosure (e.g., the computer-implemented methods and other information processing methods described herein: method 200 of FIG. 2A through 2E; etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 192 optionally stores a subset of the modules and data structures identified above. Furthermore, in some embodiments, the memory 192 stores additional modules and data structures not described above.

It should be appreciated that the computer system 100 of FIGS. 1A and 1B is only one example of a computer system 100, and that the computer system 100 optionally has more or fewer components than shown, optionally combines two or more components, or optionally has a different configuration or arrangement of the components. The various components shown in FIGS. 1A and 1B are implemented in hardware, software, firmware, or a combination thereof, including one or more signal processing and/or application specific integrated circuits.

Now that a general topology of the distributed system 100 has been described in accordance with various embodiments of the present disclosures, details regarding some processes in accordance with FIGS. 2A through 2E will be described.

FIGS. 2A through 2E illustrate a flow chart of methods (e.g., method 200) for identifying one or more combinatorial substitutions that affect one or more properties (e.g., a first property, a second property, etc.) of a target protein, such as by training a surrogate model within an N-dimensional space, in accordance with embodiments of the present disclosure. Specifically, an exemplary method 200 for identifying one or more combinatorial substitutions that affect such one or more properties of a target protein is provided, in accordance with some embodiments of the present disclosure. In the flow charts, the preferred parts of the methods are shown in solid line boxes, whereas optional variants of the methods, or optional equipment used by the methods, are shown in dashed line boxes.

Various modules in the memory 192 of the computer system 100 (e.g., protein library 106, model library 114, client application 120, or a combination thereof of FIGS. 1A and 1B), the memory 192 of the computer system 100, or both perform certain processes of the methods 200 described in FIGS. 2A through 2E, unless expressly stated otherwise. Furthermore, it will be appreciated that the processes in FIGS. 2A through 2E can be encoded in a single module or any combination of modules.

Block 202. Referring to block 202 of FIG. 2A, a method 200 for identifying one or more combinatorial substitutions that affect one or more properties of a target protein is provided.

In some embodiments, the one or more combinatorial substitutions includes at least one combinatorial substitution, at least 2 combinatorial substitutions, at least 5 combinatorial substitutions, at least 10 combinatorial substitutions, at least 15 combinatorial substitutions, at least 20 combinatorial substitutions, at least 25 combinatorial substitutions, at least 35 combinatorial substitutions, at least 45 combinatorial substitutions, at least 50 combinatorial substitutions, at least 60 combinatorial substitutions, at least 75 combinatorial substitutions, at least 100 combinatorial substitutions, at least 125 combinatorial substitutions, at least 150 combinatorial substitutions, at least 175 combinatorial substitutions, at least 200 combinatorial substitutions, at least 225 combinatorial substitutions, at least 250 combinatorial substitutions, at least 300 combinatorial substitutions, at least 500 combinatorial substitutions, where each such combinatorial substitution is the substitution of one position in the target protein away from a reference sequence. In other words each combinatorial substitution is independently αXXβ, where XX is a position in the reference sequence for the target protein, α is the identity of the amino acid at reference position XX, and β is the identity of the single amino acid substitution at reference position XX, and where XX is an integer in the set 1 to N, where N is the number of residues in the reference sequence for the target protein.

In some embodiments, the reference sequence for the target protein is a native sequence of a naturally occurring gene or a portion thereof.

In other embodiments, the reference sequence is in fact a sequence that contains a number of mutations, in the form of point mutations, insertions, deletions, the fusion of multiple naturally occurring proteins or portions thereof, or any combination thereof. In such embodiments, this remains the reference sequence on the basis that each of the proteins evaluated for the target protein have this reference sequence, with the exception of one or more mutations introduced using the systems and methods of the present disclosure.

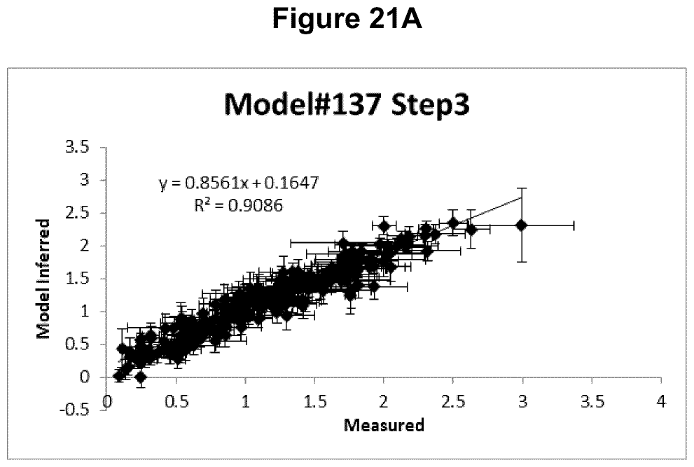

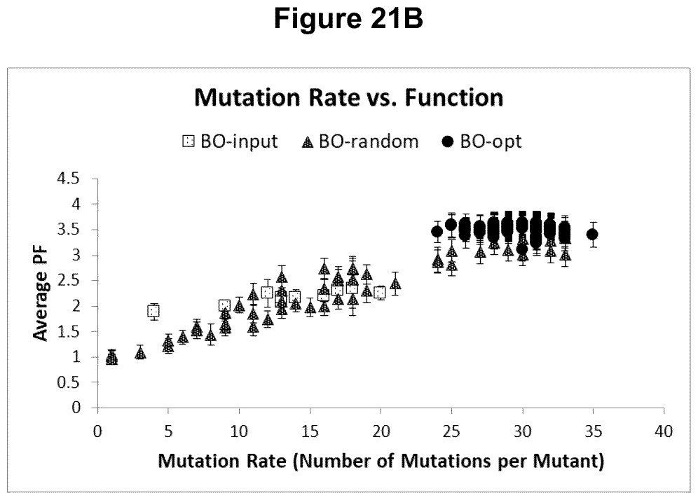

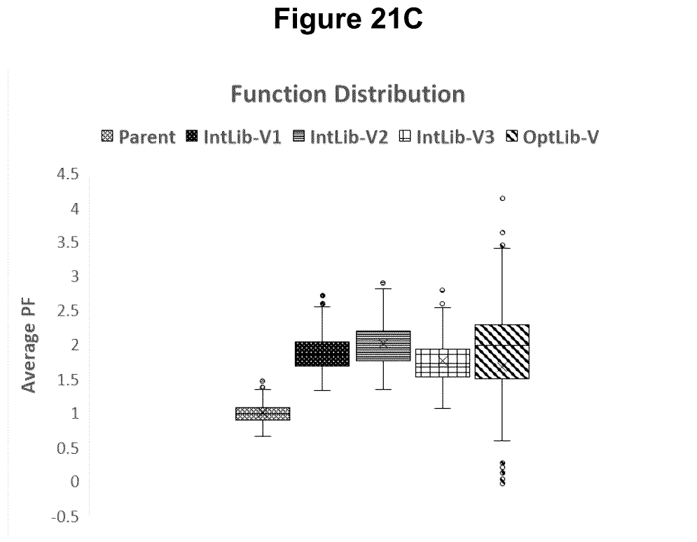

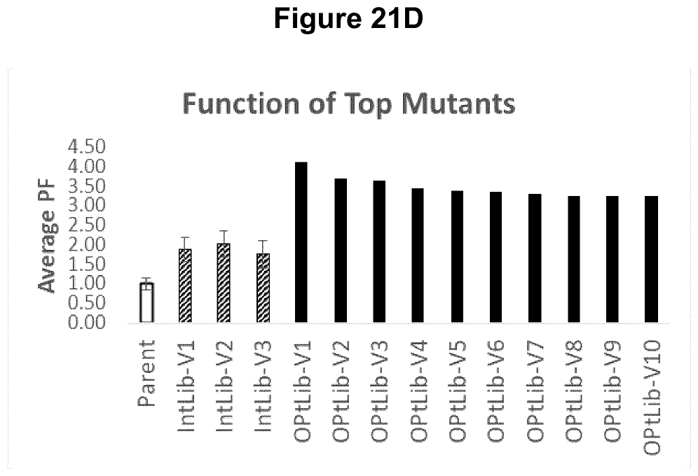

In some embodiments, the method 200 is implemented at a computer system (e.g., computer system 100 of FIGS. 1A and 1B). The computer system includes one or more processors (e.g., CPU 174 of FIGS. 1A and 21B) and a memory (e.g., memory 192 of FIGS. 1A and 1B) coupled to the one or more processors 174. The memory 192 includes one or more programs (e.g., protein library 106, model library 114, client application 120, or a combination thereof of FIGS. 1A and 1B) configured to be executed by the one or more processors 174. Accordingly, in such embodiments, the one or more programs, when executed by the one or more processors, perform the method 200. As such, portions of the method 200 require a computer (e.g., computer system 100 of FIGS. 1A and 1B) to be used because the considerations used by the systems and methods of the present disclosure, on the scale performed by the systems and methods of the present disclosure, cannot be mentally performed. In other words, given an input to a model 116 to collectively consider each respective result (e.g., the first property 112-1 of the target protein 108), the model 116 output (e.g., the one or more combinatorial substitutions) needs to be determined using the computer rather than mentally in such embodiments.

Block 204. Referring to block 204, in some embodiments, the first property of the target protein 108 is a functional property, a genomic property, or a therapeutic property that affects a biochemical and/or structural aspect of the target protein 108.

For instance, in some embodiments, the first property of the target protein 108 is a solubility of the target protein 108, an ability of the target protein 108 to carry out an enzymatic activity (e.g., in a predetermined pH range), an aliphatic index of the target protein 108, a molecular weight of the target protein 108, a charge of the target protein 108, an isoelectric point of the target protein 108, or a viscosity of the target protein 108.

Exemplary techniques for measuring viscosity of substances such as proteins, and the types of viscosity that can be measured are described in Malcom, 2002, Food Texture and Viscosity, Second Edition, Chapter 6 “Viscosity Measurement,” pp. 235-256, Elsevier Inc., and (W. Boyes, ed.), 2009, Instrumentation Reference Book, Fourth Edition, Chapter 7, pp. 69-75, “Measurement of Viscosity,” each of which is hereby incorporated by reference.

In some embodiments, the first property 112-1 of the target protein is a functional property of a protein such as emulsification ability, water binding ability, swelling ability, phase separation, oil holding capacity, foaming ability, coalescence ability, gelling ability, film formation ability, gelation ability, caramelization ability, aeration ability, chewiness, gumminess, springiness, sensory (taste, texture, flavor, aroma, mouthfeel, aftertaste, finish, appearance), syneresis, cohesiveness, brittleness, elasticity, adhesiveness, shelf-life, color, and odor.

In some embodiments, the first property 112-1 is a therapeutic property. Non-limiting examples of therapeutic properties include, but are not limited, an ability to degrade glycogen (e.g., as demonstrated by alglucosidase-α), an ability to digest glycosaminoglycans within lysosomes (e.g., as demonstrated by laronidase), an ability to cleave O-sulfates thereby preventing glycoseaminoglycan accumulation (e.g., as demonstrated by idursulfase), an ability to cleave terminal sulphages from glycoseaminoglycans (e.g., as demonstrated by galsulfase), an ability to hydrolyze glycosphingolipids (e.g., as demonstrated by agalsidase-β), an ability to digest lactose (e.g., as demonstrated by lactase), an ability to digest food (e.g., as demonstrated by pancreatic enzymes such as lipase and amylase), an ability to metabolize adenosine (e.g., as demonstrated by adenosine deaminase), an ability to break down blood clots (e.g., as demonstrated by tissue plasminogen activator), an ability to cause blood to clot (e.g., as demonstrated by Factor VIIa), an ability to hydrolyze proteins (e.g., as demonstrated by serine proteases such as drotrecogin-α and trypsin), an ability to inactivate SNAP-25 (e.g., as demonstrated by botulinum toxin type A and by botulinum toxin type B), an ability to digest native collagen (e.g., as demonstrated by collagenase), an ability to cleave DNA (e.g., as demonstrated by human deoxyribonuclease I), an ability to hydrolyze hyaluronan (e.g., as demonstrated by hyaluronidase), an ability to hydrolyze proteins (e.g., as demonstrated by cysteine proteases such as papain), an ability to catalyze the conversion of L-asparagine to aspartic acid and ammonia (e.g., as demonstrated by L-Asparaginase), an ability to catalyze the conversion of uric acid to allantoin (e.g., as demonstrated by urate oxidases such as rasburicase), an ability to regulate glucose in humans (e.g., as demonstrated by insulin and pramlintide acetate), an ability to stimulate human growth (e.g., as demonstrated by human growth hormone and mecasermin), anti-coagulation (e.g., as demonstrated by Protein C), erythropoiesis stimulation (e.g., as demonstrated by erythropoietin), neutrophil proliferation (e.g., as demonstrated by granulocyte colony-stimulating factor), an ability to stimulate granulocytemacrophages (e.g., as demonstrated by granulocy temacrophage colony-stimulating factor), treatment of cancer (e.g., as demonstrated by the treatment of chronic lymphocytic leukemia by ofatumumab and also demonstrated by the treatment of Metastatic melanoma by ipilimuma), treatment of bone loss (e.g., as demonstrated by denosumab), treatment of system lupus erythematosus (e.g., as demonstrated by Belimumab), treatment of Anthrax infection (e.g., as demonstrated by raxibacumab), treatment of Hodgkin lymphoma (e.g., as demonstrated by Brentuximab vedotin), treatment of diabetes (e.g., as demonstrated by insulin glargine, insulin aspart, rhu insulin, and insulin lispro), treatment of multiple sclerosis (e.g., as demonstrated by Interferon beta-1a), and treatment of anemia (e.g., as demonstrated by epoetin beta). See, for example, Dimitrov. 2012, “Therapeutic Proteins,” Methods Mol. Biol. 899, pp. 1-26, which is hereby incorporated by reference.

Accordingly, the method 200 allows for identifying the one or more combinatorial substitutions that affect the first property 112-1 of the target protein. In some embodiments, the goal is to affect the first property of the target protein by increasing or decreasing a metric representative of the first property (e.g., increasing or decreasing the solubility of the target protein 108, increase disease fighting ability, etc.). In some embodiments, the goal is to affect the first property of the target protein by removing the first property altogether from the target protein.

Blocks 206-208. Referring to block 206, in some embodiments, the target protein 108 is an enzyme. Accordingly, in some such embodiments, the first property of the target protein 108 is an enzymatic activity of the target protein 108. Referring to block 208, examples of enzymatic activity classes include hydrolases, oxidoreductases, lyases, transferases, ligases, isomerases, and ligases. See, for example, 2012, Food Biochemistry and Food Processing, Second Edition, Benjamin Simpson ed., Wiley-Blackwell, Ames, Iowa, Ako and Nip, Chapter 6 “Enzyme Classification and Nomenclature,” which is hereby incorporated by reference in its entirety.

Block 210. Referring to block 210, in some embodiments, the target protein 108 is an enzyme, a co-enzyme, a structural protein, a nutrient protein, a regulatory protein, a defense protein, a transport protein, a storage protein, a contractile protein, or a toxic protein (e.g., a ribosome-inactivating protein).

Non-limiting examples of enzymes and co-enzymes are disclosed in Enzyme Technology, Pandey, Webb Soccol, and Larroche, eds., 2006, Springer New York, which is hereby incorporated by reference in its entirety.

Non-limiting examples of toxic proteins are found in Toxic Plant Proteins, Lord and Hartley eds., Plant Cell Monographs 18, 2010, Springer Berlin Heidelberg, Berlin, Germany, which is hereby incorporated by reference in its entirety.

Block 212. Referring to block 212 of FIG. 2A, in some embodiments, the target protein 108 includes 5 or more residues, 10 or more residues, 15 or more residues, 20 or more residues, 25 or more residues, 35 or more residues, 45 or more residues, 50 or more residues, 60 or more residues, 75 or more residues, 100 or more residues, 125 or more residues, 150 or more residues, 175 or more residues, 200 or more residues, 225 or more residues, 250) or more residues, 300 or more residues, or 500 or more residues. In some embodiments, the target protein includes a single functional domain, or two or more functional domains. In some embodiments, the target protein is a fusion of two or more naturally occurring proteins, or portions thereof.

Block 214. Referring to block 214, the method 200 includes obtaining an identity of each single point mutation in a first plurality of single point mutations of the target protein. In some embodiments, the first plurality of single point mutations includes at least three single point mutations, at least five single point mutations, at least ten single point mutations, at least fifteen single point mutations, at least twenty single point mutations, at least twenty-five single point mutations, at least thirty single point mutations, at least forty single point mutations, at least fifty single point mutations, at least seventy-five single point mutations, at least one hundred single point mutations, at least five hundred single point mutations, at least five thousand single point mutations, at least ten thousand single point mutations, at least fifty thousand single point mutations (e.g., a first protein 108-1 with a sequence of 2,000 amino acid that yields 38,000 possible single point mutations in comparison to a reference sequence), or a combination thereof.

Each respective single point mutation in the first plurality of single point mutations defines a corresponding single point substituted protein characterized by a reference sequence for the target protein with the exception of an alteration at a respective independent position within the reference sequence to an amino acid other than that found in the reference sequence. For instance, referring briefly to FIG. 6A, in one example, the first plurality of single point mutations includes a first single point mutation at a twenty-eighth position (e.g., N28E), a second single point mutation at a forty-first position (G41S), a third single point mutation at a seventy-first position (e.g., D71P), etc. The sequence of each respective single point substituted protein is identical to the starting reference sequence except for 1 position, and the substitution occurring at this 1 position defines or characterizes the respective single point substituted protein. Using the nomenclature αXXβ, where XX is a position in the reference sequence for the respective single point substituted protein, α is the identity of the amino acid at position XX in the reference sequence for the respective single point substituted protein, and β is the identity of the single amino acid substitution at position XX, each respective single point substituted protein includes one substitution of form αXXβ. It is possible for two or more different single point substituted protein to have a substitution at the same position XX in the reference sequence. However, in such instances, each of these single point substituted proteins will have a different amino acid substitution at this position XX. In other words each of these single point substituted proteins will have a different B.

Block 216. Referring to block 216 of FIG. 2B, the method 200 includes obtaining a corresponding set of values for a set of properties (e.g., properties 112) of the corresponding point 110 substituted protein for each corresponding point 110 substituted protein defined by the first plurality of single point 110 mutations. In some embodiments, the corresponding set of values for the set of properties 112 has a one-to-one relationship, such that each respective property 112 in the set of properties 112 has one corresponding value in the corresponding set of values. However, the present disclosure is not limited thereto. For instance, in some embodiments, the corresponding set of values for the set of properties 112 has a many-to-one relationship, such that each respective property 112 in the set of properties 112 has an array of values in the corresponding set of values. Furthermore, in some embodiments, the set of properties 112 includes at least two properties (e.g., stability property and binding property), at least three properties 112 (e.g., stability property, binding property, and at least one protein formulation property), at least 5 properties 112 (e.g., stability property, binding property, and at least three protein formulation properties), at least 10 properties 112, at least 15 properties 112, at least 25 properties 112, at least 50 properties 112, or a combination thereof. As a non-limiting example, in some embodiments, the corresponding set of values for the set of properties 112 of the corresponding point substituted protein includes various biological process properties 112 associated with protein interaction networks (e.g., mRNA processing, translational termination and/or elongation, RNA splicing, glycolysis, mitosis, acute-phase response, platelet activation, cell adhesion, etc.). By determining the corresponding set of values for the set of properties 112, the method 200 forms an N-dimensional data set that represents the limits of the set of values. In some embodiments, the set of properties 112 includes a stability of the corresponding point substituted protein. In some embodiments, the set of properties 112 includes at least one protein formulation property 112 of the corresponding point 110 substituted protein. Moreover, in some embodiments, the set of properties 112 includes a determination that the respective single point 110 mutation in the corresponding point substituted protein occurs at a predetermined position that exhibits variability across a plurality of naturally occurring homologs of the target protein 108. In some embodiments, the homologs of the target protein 108 is a similarity in sequence between the target protein 108 and a respective protein 108. For example, in some embodiments, the method 200 imparts a bias towards or away from a reference sequence or family of sequences of a target protein 110. As a non-limiting example, in some such embodiments, the method 200 imparts a bias towards a wild-type residue or a homolog residue. However, the present disclosure is not limited thereto.

As a non-limiting example, in some embodiments, the set of properties 112 includes the length of the corresponding point substituted protein, the molecular weight of the corresponding point substituted protein, the number of atoms of the corresponding point substituted protein, the grand average of hydropathicity (GRAVY) of the corresponding point substituted protein, the amino acid composition of the corresponding point substituted protein (e.g., the percentage of each amino acid in the target protein 108), the periodicity of the corresponding point substituted protein, a physicochemical property of the corresponding point substituted protein, the predicted secondary structure of the corresponding point substituted protein, a subcellular location of the corresponding point substituted protein, a sequence motif of the corresponding point substituted protein, or a combination thereof. However, the present disclosure is not limited thereto.

Block 218. Referring to block 218, in some embodiments, the at least one protein formulation property 112 is an electrostatic property of the corresponding point substituted protein, a developability index of the corresponding point substituted protein, a solubility of the corresponding point substituted protein, a measure of aggregation of the corresponding point substituted protein, a viscosity of the corresponding point substituted protein, or a combination thereof. As a non-limiting example, in some such embodiments, the at least protein formulation property 112 includes an amino acid composition, a hydrophobicity, a solvent accessibility, a surface tension, a charge, a polarizability, a polarity, a normalized van der Waals volume, or a combination thereof.

Block 220. Referring to block 220, in some embodiments, the set of properties 112 includes a post-translational modification that is predicted to occur to the corresponding point substituted protein. For instance, in some embodiments, the target protein 108 includes polymers that are modified by acylation, alkylation, amidation, biotinylation, formylation, γ-carboxylation, glutamylation, glycosylation, glycylation, hydroxylation, iodination, isoprenylation, lipoylation, cofactor addition (e.g., of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (e.g., arginylation), sulfation, selenoylation, ISGylation, SUMOylation, ubiquitination, chemical modifications (e.g., citrullination and deamidation), and treatment with other enzymes (e.g., proteases, phosphotases and kinases). One of skill in the art will appreciate that other types of post-translational modifications applicable to the systems and methods of the present disclosure.

Block 222. Referring to block 222, in some embodiments, the set of properties 112 further includes an immunogenicity of the corresponding point substituted protein. In some embodiments, immunogenicity of the corresponding point substituted protein is determined using the IEDB immunogenicity predictor with a particular HLA type (http://tools.immuneepitope.org/immunogenicity/) or CTLPred (http://www.imtech.res.in/raghava/ctlpred/). In some embodiments, the immunogenicity of the corresponding point substituted protein is based upon calculated immunogenicity of a peptide centered on the position of the point substituted protein. For instance, in some embodiments, the immunogenicity of the corresponding point substituted protein is calculated using a peptide that includes the point substituted position and the X 5′ flanking residues and the Y 3′ flanking residues of the point substituted position, where X and Y are each independent positive integers. In other embodiments, the immunogenicity of the corresponding point substituted protein is calculated using the entire sequence of the corresponding point substituted protein.

Block 224. Referring to block 224, in some embodiments, the set of properties 112 includes a binding energy of the corresponding point substituted protein. In some embodiments, this binding energy is a calculated binding energy of the corresponding point substituted protein to a particular compound. In some embodiments, this binding energy is the score provided by a docking program to the docking of the particular compound to the corresponding point substituted protein. Example docking programs include, but are not limited to Jones et al., 1995, “Molecular recognition of receptor sites using a genetic algorithm with a description of desolvation,” J Mol Biol 245, pp. 43-53; Jones et al., 1997, Development and validation of a genetic algorithm for flexible docking. J Mol Biol 267, pp. 727-748; Ewing et al., “DOCK 4.0: search strategies for automated molecular docking of flexible molecule databases,” J Comput Aided Mol Des 15, pp. 411-428: Goodsell et al., 1996, “Automated docking of flexible ligands: applications of AutoDock,” J Mol Recognit 9, pp. 1-5: Friesner et al., 2004, “Glide: a new approach for rapid, accurate docking and scoring, “Method and assessment of docking accuracy,” J Med Chem 47: 1739-1749; Halgren et al., 2004, “Glide: a new approach for rapid, accurate docking and scoring. 2. Enrichment factors in database screening,” J Med Chem 47, pp. 1750-1759; Rarey et al., 1996, “A fast flexible docking method using an incremental construction algorithm,” J Mol Biol 261, pp. 470-489; and Trott Olson, 2010, “AutoDock Vina: improving the speed and accuracy of docking with a new scoring function, efficient optimization and multithreading.” J Comput Chem 31, pp. 455-461, each of which is hereby incorporated by reference.

In some embodiments, the calculated binding energy of the corresponding point substituted protein to a particular compound is the score provided by an all-atom molecular dynamics (MD) simulation with explicit solvent, in combination with efficient and rigorous free energy calculation methods such as, for example, disclosed in Gilson and Zhou, 2007, “Calculation of protein-ligand binding affinities,” Annu Rev Biophys Biomol Struct 36, pp. 21-42, which is hereby incorporated by reference. In some alternative embodiments, the binding energy of the corresponding point substituted protein to a particular compound is calculated using a linear response approximation (see, for example, Lee et al., 1992, “Calculations of antibody antigen interactions-microscopic and semimicroscopic evaluation of the free energies of binding of phosphrycholine analogs to Mcpc603,” Protein Eng 5, pp. 215-228, which is hereby incorporated by reference) or a linear interaction energy (see, for example. Aqvist et al., 1994. “New method for predicting binding-affinity in computer-aided drug design,” Protein Eng 7: 385-391, which is hereby incorporated by reference), where only the ligand-bound and unbound states are simulated. In some embodiments the calculated binding energy of the corresponding point substituted protein to a particular compound is calculated using a semimacroscopic approach based on protein dipoles Langevin dipoles (PDLD/S) and LRA (PDLD/S-LRA) thereby reducing the computational cost without loss of accuracy (see, for example, Sham et al., 2000, “Examining methods for calculations of binding free energies: LRA, LIE, PDLD-LRA, and PDLD/S-LRA calculations of ligands binding to an HIV protease,” Proteins 39, pp. 393-407, and Singh and Warshel, 2010, “Absolute binding free energy calculations: on the accuracy of computational scoring of protein-ligand interactions,” Proteins 78, pp. 1705-1723, each of which is hereby incorporated by reference. In some embodiments, the binding energy of the corresponding point substituted protein to a particular compound is calculated using molecular mechanics-Poisson Boltzmann (or Generalized Born) surface area (MM-PB(GB)SA) methods. See, for example, Kollman et al., 2000, “Calculating structures and free energies of complex molecules: Combining molecular mechanics and continuum models,” Acc Chem Res 33: 889-897, and Gohlke and Case, 2004, “Converging free energy estimates: MM-PB(GB)SA studies on the protein-protein complex Ras-Raf,” J Comput Chem 25, pp. 238-250, each of which is hereby incorporated by reference.

Block 226. Referring to block 226 of FIG. 2B, in some embodiments, the set of properties 112 includes the stability of the corresponding point substituted protein. In some such embodiments, the stability of the corresponding point substituted protein is determined using one or more crystal structures or atomistic models 116 of the target protein 108, modified to include the point mutation of the corresponding point substituted protein. For instance, referring briefly to FIG. 3, a mutation energy stability property of the corresponding point substituted protein is determined using 3WP4, 3WP5, and 3WP6 crystal strictures for acid xylanases at pH 2.5 and a temperature of 37 degrees Celsius. In some such embodiments, the one or more crystal structures or atomistic models 116 of the target protein 108, modified to include the corresponding point substituted protein, provide determinations of one or more physical principles and thermodynamic forces associated with the corresponding point substituted protein, and such determinations are included in the set of properties 112 of the corresponding point substituted protein. However, the present disclosure is not limited thereto. For example, in some embodiments such models are used to compute the solvent-accessible surface area (SASA) of the corresponding point substituted protein and the SASA is included in the set of properties 112 of the corresponding point substituted protein. Example algorithms for computing SASA include, but are not limited to, those disclosed in Lee and Richards, 1971, “The interpretation of protein structures: estimation of static accessibility,” J Mol Biol. 55(3), pp. 379-400, which is hereby incorporated by reference. As another example, in some embodiments such models are used to compute the solvent-excluded surface area (Connolly surface) of the corresponding point substituted protein and this is included in the set of properties 112 of the corresponding point substituted protein. Example algorithms for computing the Conolly surface include, but are not limited to, those disclosed in Connolly, 1993, “The molecular surface package,” J Mol Graphics. 11(2), pp. 139-141 which is hereby incorporated by reference.

Block 232. Referring to block 228 of FIG. 2C, the method 200 includes filtering the first plurality of single point mutations to form a second plurality of single point mutations based at least upon each corresponding set of values for each corresponding set of properties 112 for each corresponding point substituted protein. By filtering the first plurality of single point mutations, the method 200 creates a data set in the form of the second plurality of single point mutations that optimizes tradeoffs between exploration of the single point mutations and exploitation of the variability between respective point mutations, such as by removing deleterious or redundant point mutations from the first plurality of single point mutations. Accordingly, the second plurality of single point mutations includes less than all of the first plurality of single point mutations. In some embodiments, the second plurality of single point mutations includes at least three single point mutations, at least five single point mutations, at least ten single point mutations, at least fifteen single point mutations, at least twenty single point mutations, at least twenty-five single point mutations, at least thirty single point mutations, at least forty single point mutations, at least fifty single point mutations, at least seventy-five single point mutations, at least one hundred single point mutations.

In some embodiments, the corresponding set of values 112 used in the filtering includes at least, for each corresponding point substituted protein representing a point mutation in the first plurality of point mutations, a determination of the mutation energy stability of the corresponding point substituted protein, the mutation energy binding of the corresponding point substituted protein, a determination of non-severed point positions of the corresponding point substituted protein, a determination of allowed mutations in one or more homologs of the target protein 108, or a combination thereof. Accordingly, in some embodiments, this filtering is configured to force diversity within the second plurality of single-point mutations, such as filtering out a first point mutation 110-1 from the first plurality of single point mutations based on desire to sample a greater portion of the sequence of the target protein 108. Accordingly, in some such embodiments, the filtering includes determining, for each corresponding point substituted protein defined by the first plurality of single point mutations, for each respective property in the set of properties, whether a value of the respective property 112 in the corresponding set of values for the corresponding point substituted protein satisfies a corresponding threshold value requirement for the respective property 112. The corresponding point substituted protein is included in the second plurality of single point mutations when each corresponding threshold value requirement for each property in the set of properties is satisfied, and the corresponding point substituted protein is not included in the second plurality of single point mutations when any corresponding threshold value requirement of any property in the set of properties is not satisfied.

However, the present disclosure is not limited thereto. For example, consider the case in which the set of properties includes five different properties 112. Each of these five different properties will have its own threshold value requirement. Thus, in order for a point mutation to be included in the second plurality of point mutations, the value of each respective property of the five properties of the point substituted protein must satisfy the corresponding threshold value requirement of the respective property.

Referring to blocks 230 and 232, in some embodiments, the corresponding threshold value for one of the properties in the set of properties, stability, is a particular stability value. In some such embodiments, the particular stability value is a calculated stability of the target protein (e.g., using a crystal structure or atomic model of the target protein). As a non-limiting example, in some embodiments, the particular stability value of the corresponding threshold value is a mutation energy stability (e.g., stability value in Calories per mole). In such embodiments, when the corresponding point substituted protein has a stability that is better (e.g., block 232) or is at least a threshold percentage of or better (e.g., block 234) than the stability of the target protein 108, the corresponding point substituted protein satisfies this property. If the corresponding point substituted protein satisfies the threshold requirements of all the other properties in the set of properties, it is included in the second plurality of single point mutations. Moreover, when the corresponding point substituted protein has a stability that is worse (e.g., block 232) or not within a threshold percentage (e.g., block 234) than the stability of the target protein, the corresponding point substituted protein is not included in the second plurality of single point mutations. In some embodiments, the stability of the corresponding point substituted protein is considered better than the stability of the target protein 108 when the calculated value for the stability of the corresponding point substituted protein is greater than the calculated value for the stability of the target protein 108. However, the present disclosure is not limited thereto. With reference to block 234, in some embodiments the threshold percentage is a particular percentage selected from the range of 65 percent to 100 percent, 70 percent to 100 percent, 75 percent to 100 percent, 80 percent to 100 percent, 85 percent to 100 percent, 90 percent to 100 percent, 95 percent to 100 percent, or 97 percent to 100 percent. For example, in some embodiments where the range is 70 percent to 100 percent, the particular threshold percentage is 80 percent. When the threshold percentage is 80 percent, the corresponding point substituted protein must have at least 80 percent of the calculated stability of the target protein in order to satisfy the stability threshold requirement.

Block 234-236. Referring to block 234 of FIG. 2D, the method 200 provides for selecting a first plurality of combinatorially substituted proteins. Each respective combinatorially substituted protein in the first plurality of combinatorially substituted proteins is characterized by the reference sequence for the target protein 108 with the exception of the independent inclusion of two or more single point mutations from the second plurality of single point mutations. Said otherwise, in some such embodiments, each respective combinatorially substituted protein in the first plurality of combinatorially substituted proteins is characterized by a reference protein 108 sequence, except for the independent inclusion of two or more single point mutations from the second plurality of single point mutations. The goal of the selection of such single point mutations in forming the first plurality of combinatorially substituted proteins is to sample the combinatorial space defined by the second plurality of single point mutations. It is not feasible for the first plurality of combinatorially substituted proteins to exhaustively include every possibility defined by the combinatorial space defined by the second plurality of single point mutations. In some embodiments, the first plurality of combinatorially substituted proteins represents less than 5 percent, less than 3 percent, less than 1 percent, less than 0.1 percent, less than 0.01 percent, less than 0.001 percent, less than 0.0001 percent, or less than 0.00001 percent of the possible number of combinatorially substituted proteins that could be drawn from the second plurality of single point mutations.

Referring to block 236 of FIG. 2D, in some embodiments, each combinatorially substituted protein in the first plurality of combinatorially substituted proteins includes three or more point substitutions, four or more point substitutions, five or more point substitutions, six or more point substitutions point substitutions, seven or more point substitutions, eight or more point substitutions, nine or more point substitutions, ten or more point substitutions, fifteen or more point substitutions, twenty or more point substitutions, twenty five or more point substitutions, forty or more point substitutions, fifty or more point substitutions, or a hundred or more point substitutions drawn from the second plurality of single point mutations. Accordingly, in some embodiments, each combinatorially substituted protein in the first plurality of combinatorially substituted proteins includes between three and one hundred or more point substitutions drawn from the second plurality of single point mutations.

It is possible that the first property that is measured in accordance with blocks 234-236 is the same as one of the properties that was used in the set of properties used to filter the first plurality of mutations into the second plurality of mutations. However, in typical embodiments, the set of properties used to filter the first plurality of mutations into the second plurality of mutations are determined in silico whereas the property that is measured for each combinatorially substituted protein in the first plurality of combinatorially substituted proteins is physically measured. Moreover, in typical embodiments, the first property that is measured in accordance with blocks 234-236 for each combinatorially substituted protein in the first plurality of combinatorially substituted proteins is, in fact, a property that the disclosed systems and methods seeks to optimize for the target protein. In some embodiments, the property that the disclosed systems and methods seeks to optimize for the target protein cannot be directly measured and the first property that is measured in accordance with blocks 234-236 for each combinatorially substituted protein in the first plurality of combinatorially substituted proteins is a proxy for the property that the disclosed systems and methods seeks to optimize for the target protein.

In some embodiments, the first property that is measured in accordance with blocks 234-236 is a measurement by fluorescence spectroscopy (absorption, excitation, or emission) of each combinatorially substituted protein in the first plurality of combinatorially substituted proteins. Such spectroscopic techniques are disclosed in Physical Methods to Characterize Pharmaceutical Proteins, Herron, Jiskoot, and Crommelin, eds., Springer Science+Business Media New York, 1995, Chapter 1 entitled “Application of Fluorescence Spectroscopy for Determining the Structure and Function of Proteins,” which is hereby incorporated by reference.

In some embodiments, the first property that is measured in accordance with blocks 234-236 is a measurement by circular dichroism of each combinatorially substituted protein in the first plurality of combinatorially substituted proteins. Such spectroscopic techniques are disclosed in Physical Methods to Characterize Pharmaceutical Proteins, Herron, Jiskoot, and Crommelin, eds., Springer Science+Business Media New York, 1995, Chapter 2 entitled “Structural Information on Proteins from Circular Dichroism Spectroscopy: Possibilities and Limitations,” which is hereby incorporated by reference.

In some embodiments, the first property that is measured in accordance with blocks 234-236 is a measurement by nuclear magnetic resonance of each combinatorially substituted protein in the first plurality of combinatorially substituted proteins. Such spectroscopic techniques are disclosed in Physical Methods to Characterize Pharmaceutical Proteins, Herron, Jiskoot, and Crommelin, eds., Springer Science+Business Media New York, 1995, Chapter 3 entitled “Two-, Three-, and Four-Dimensional Nuclear Magnetic Resonance Spectroscopy of Protein Pharmaceuticals,” which is hereby incorporated by reference.

In some embodiments, the first property that is measured in accordance with blocks 234-236 is a measurement of a binding coefficient, expressed for example, as a IC50, EC50 or KI, of each combinatorially substituted protein in the first plurality of combinatorially substituted proteins to a particular compound using a wet lab binding assay.

In some embodiments, the target protein is an enzyme and the first property is a characterization of the enzymatic property. For instance, in some embodiments, the enzymatic property is measured with respect to a natural substrate of the target protein. In some such embodiments, the first property is a rate constant k, an acid dissociation constant Kₐ, a competitive-inhibition constant Kⱼ, an uncompetitive-inhibition constant Kᵢ, a Michaelis constant Kₘ, an apparent value of Kₘ, an expected value of Kₘ, a substrate-inhibition constant Kₛᵢ, a catalytic constant k_{cat}, a rate of reaction ν, a free energy of activation, a maximum velocity V, a standard enthalpy of reaction, an enthalpy of activation, an entropy of activation, or a relation time that is measured or determined from measurements for each combinatorially substituted protein in the first plurality of combinatorially substituted proteins. Such properties are discussed in further detail in Cornish-Bowden, Fundamental of Enzyme Kinetics, 1979, The Butterworth Inc., Boston, Massachusetts, which is hereby incorporated by reference.

Block 240. Referring to block 240, in some embodiments, the first property measured for each combinatorially substituted protein in the first plurality of combinatorially substituted proteins from block 234 serves as a training label against the identity of the point substitutions in each combinatorially substituted protein in the first plurality of combinatorially substituted proteins to train a surrogate model (e.g., first model 116-1 of FIG. 1B), such as training the surrogate model 116 to identify one or more candidate features (e.g., a first vector feature associated with the first property of the target protein 108). Thusly, the surrogate model 116 is a data-drive model 116 that is used to predict the identity of the combinatorially substituted proteins. In some embodiments, the prediction includes tuning one or more parameters (e.g., weights) that is combined with extracted features (e.g., identified combinatorially substituted proteins). Said otherwise, once the corresponding measured value of the first property for each combinatorially substituted protein in a first plurality of combinatorially substituted proteins is obtained (e.g., block 234 of FIG. 2D), the surrogate model 116 provides a mapping (e.g., pairing) between the input features and data points of the identity of each single point mutation in the respective combinatorially substituted protein and the corresponding measured value of the first property 112-2.

In some embodiments, by utilizing the surrogate model 116, the method 200 a provides probabilistic determination of the identities of the one or more combinatorial substitutions that affect the first property 112-1 of the target protein. More particularly, in some embodiments, the surrogate model 116 tunes parameters in order to provide a solution that leads to this identification given an input of the first property 112-1 (e.g., a finite data set). From this determination of the identities of the one or more combinatorial substitutions that affect the first property 112-1 of the target protein, the surrogate model 116 provides an approximation of this function to tune one or more parameters and/or hyperparameters of a posterior of the surrogate model 116 for identifying the one or more combinatorial substitutions that affect the first property 112-1 of the target protein. This tuning of the surrogate model 116 is based on the corresponding measured value of the first property in the respective combinatorically substituted proteins. Accordingly, in some embodiments, the surrogate model 116 utilizes the corresponding measured value of the first property for each combinatorially substituted protein in the first plurality of combinatorially substituted proteins to determine an affect of the first property of the target protein. Said otherwise, in some such embodiments, the surrogate model 116 is trained against pairs of tuned parameters of the surrogate model 116 and the corresponding measured value of the first property 112-1 in the respective combinatorially substituted proteins.

This training of the surrogate model 116 is conducted within an N-dimensional space. The N-dimensional space is a mathematical construct (e.g., data set) having N-dimensions. Said otherwise, in some embodiments, the N-dimensional space is the mathematical construct in which the N-dimensions is finite and defined, at least in part, by the identity of each single point mutation in the respective combinatorially substituted protein, or dimension reduction components thereof, and the corresponding measured value of the first property 112-1. Accordingly, the surrogate model 116 provides an approximation for optimizing the effect of the first property 112-1 of the target protein within the N-dimensional space. In this way, N is a positive integer. As a non-limiting example, in some embodiments, N is a positive integer of 5 or greater, 10 or greater, 15 or greater, 20 or greater, 25 or greater, 35 or greater, 40) or greater, 50) or greater, 60 or greater, 75 or greater, 100 or greater, 125 or greater, 150) or greater, 175 or greater, 200 or greater, 250 or greater, 300 or greater, 400 or greater, 500 or greater, 750 or greater, 1,000 or greater, or a combination thereof. In some embodiments, the N-dimensional space represents each respective data element (e.g., value) within a feature space as a feature vector. Accordingly, in some such embodiments, both the N-dimensional space and each respective feature vector have N-dimensions, such as a X-axis representation of a first parameter, a Y-axis representation of a second parameter, and a Z-axis representation of a third parameter. In some embodiments, the third parameter is orthogonal to both the first parameter and the second parameter. Accordingly, due to the complexity of the N-dimensional space, in some embodiments, many redundant and/or irrelevant features are in the N-dimensional space that require addressing in order to improve results for identifying one or more combinatorial substitutions that affect the first property 112-1 of the target protein 108. In some embodiments, each of the dimensions represents a unique point substitution found in one or more of the combinatorially substituted proteins. In other embodiments, each of the dimensions represents a dimension reduction component across some combination of the unique point substitutions found in one or more of the combinatorially substituted proteins. In still other embodiments, each of the dimensions is described below in conjunction with blocks 242 and 244.

In some such embodiments, the surrogate model 116 is trained using at least the corresponding measured value of the first property 112-1 in the respective combinatorially substituted proteins against an identity of each single point mutation in the respective combinatorially substituted protein for each respective combinatorially substituted protein in the first plurality of combinatorially substituted proteins. Accordingly, the surrogate model 116 is used to determine optimal states, or protein sequences, within the N-dimensional state based on the first property of the target protein. By training the surrogate model 116 using the corresponding measured value of the first property 112-1 in the respective combinatorially substituted proteins, the method 200 determines a probability for the identity of the one or more combinatorial substitutions that affect the first property of the target protein within the N-dimensional space without human interference. In this way, the surrogate model 116 determines locations within the N-dimensional space that correspond to regions of high probability for determining the identity of the one or more combinatorial substitutions that affect the first property of the target protein. In some embodiments, the model 116 includes 20 or more parameters 118 (e.g., first parameter 118-1, third parameter 118-3, . . . , parameter Y 118-Y of model X 116-X of FIG. 1B). In some embodiments, the surrogate model comprises 40 or more parameters, 60 or more parameters, 80 or more parameters, 100 or more parameters, 200 or more parameters, 500 or more parameters, 1000 or more parameters, 10,000 or more parameters, 100,000 or more parameters, or 1×10⁶ or more parameters.

In some embodiments, the first plurality of combinatorially substituted proteins includes 20 or more proteins. In some embodiments, the first plurality of combinatorially substituted proteins consists of between 5 and 100 proteins. In some embodiments, the first plurality of combinatorially substituted proteins comprises more than 5, 10, 15, 20, 25, 30, 35, 40, 45, 50, 75, 100, 150, or 200 proteins.

Accordingly, by training the surrogate model 116 within the N-dimensional space using the corresponding measured value of the first property 112-1 in the respective combinatorially substituted proteins against the identity of each single point mutation in the respective combinatorially substituted protein for each respective combinatorially substituted protein in the first plurality of combinatorially substituted proteins, the surrogate model 116 considers only those mutants that optimally affect the first property 112-2 of the target protein to ensure the identifying one or more combinatorial substitutions is provided with a high degree of confidence. This approach of the method 200 differs from conventional techniques that assume a larger pre-existing set of proteins with the desired property 112 for training.

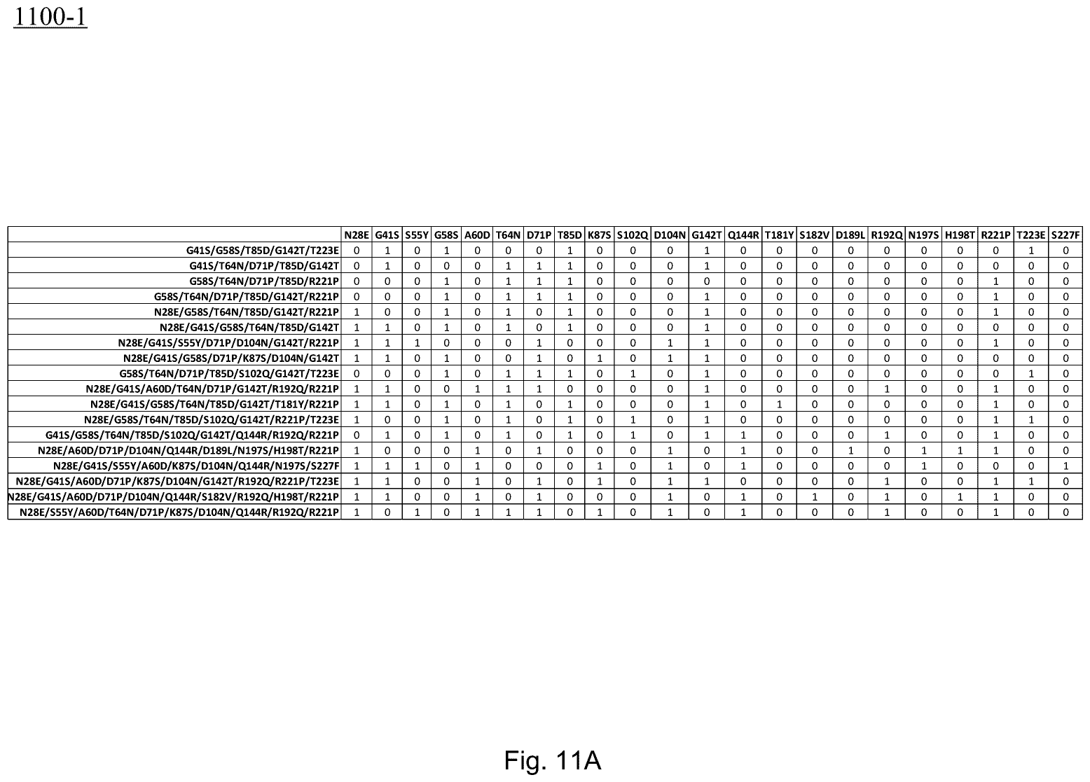

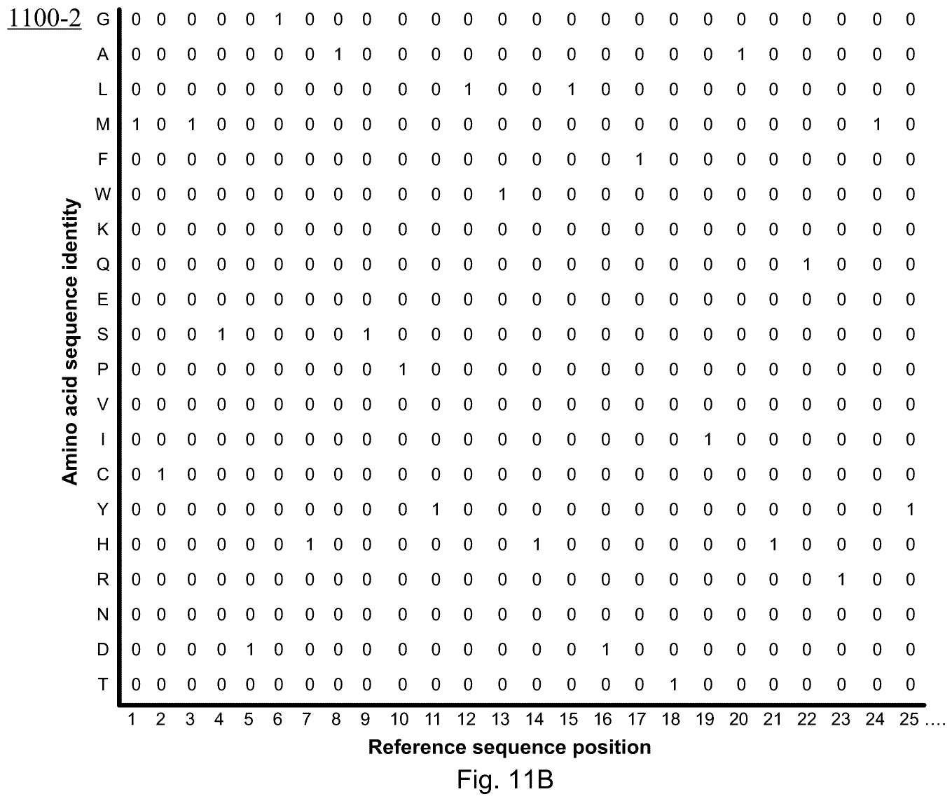

Blocks 242-244. Referring to block 242 of FIG. 2D, in some embodiments, the training of the surrogate model 116 in accordance with block 240 includes encoding each respective combinatorially substituted protein in the first plurality of combinatorially substituted proteins as an identity of each single point mutation in the respective combinatorially substituted protein in a first dimension. By encoding each respective combinatorially substituted protein in the first plurality of combinatorially substituted proteins, the method 200 provides a numerical data set that represents the mutation data of the first plurality of combinatorially substituted proteins using a numerical nomenclature. Furthermore, this encoding of each respective combinatorially substituted protein in the first plurality of combinatorially substituted proteins requires using the computer system 100, such that the method 200 cannot be performed mentally. For instance, in some embodiments, the encoding of each respective combinatorially substituted protein in the first plurality of combinatorially substituted proteins maps the sequence of each respective combinatorially substituted protein as a numerical representation of one or more dimensions (e.g., two dimensions, five dimensions, ten dimensions, fifty dimensions, a hundred dimensions, a thousand dimensions, five thousand dimensions, etc.). In some such embodiments, this encoding of each respective combinatorially substituted protein is two-dimensional, with the residue positions of the respective combinatorially substituted protein in a first dimension (e.g., columns of a two-dimensional matrix) and the sequence of the combinatorially substituted protein 108 in a second dimension (e.g., rows of the two-dimensional matrix). As a non-limiting example, consider the two-dimensional matrix 1100-2 of FIG. 11B. The columns of the matrix 1100-2 represent the residue positions of a particular combinatorially substituted protein while the residue identity of position is represented in the rows. In the matrix 1100-2, a zero value means that the corresponding residue is not found at a given position (e.g., a particular element represented by a first row and a first column of the two-dimensional matrix). In contrast, a one value means that the corresponding residue is found at a given position. Each position in the combinatorially substituted protein must have one residue type. The residue may be the reference sequence residue type in instances where that particular position has not been mutated in the particular combinatorially substituted protein or a different residue type other than that reference sequence residue type in instances where that particular residue position has been mutated. In some embodiments, to simplify the matrix, all positions of the matrix have a zero except for the columns of the matrix that corresponding to mutated positions within the particular combinatorially substituted protein. For instance, all the values in column 1 have a zero if the first position of the particular combinatorially substituted protein is not mutated, all the values in column 2 have a zero if the second position of the particular combinatorially substituted protein is not mutated, and so forth. Accordingly, the two-dimensional matrix 1100-2 of FIG. 11B represents one respective combinatorially substituted protein. However, the present disclosure is not limited thereto. As another non-limiting example, consider the two-dimensional matrix 1100-1 of FIG. 11A. The columns of the matrix 1100-1 represent the mutant positions (e.g., each corresponding point substituted protein defined by the first plurality of single point mutation, block 216 of FIG. 2B) while each respective combinatorially substituted protein in the first plurality of combinatorially substituted proteins is represented in the rows. In the matrix 1100-1, a zero value means that the corresponding point substituted protein is not found at a given position in the respective combinatorially substituted protein (e.g., a particular element represented by a first row and a first column of the two-dimensional matrix). In contrast, a one value means that the corresponding point substituted protein is found at a given position. Furthermore, in some embodiments, the matrix 1100 is symmetric or non-symmetric (e.g., a matrix 1100 that includes a gap or distinguish one or more states of a respective amino acid).

Referring to block 244, in some embodiments, the training of the surrogate model 116 includes encoding each respective combinatorially substituted protein in the first plurality of combinatorially substituted proteins as an identity of each single point mutation in the respective combinatorially substituted protein in a first dimension, a position of each single point mutation in the respective combinatorially substituted protein in a second dimension (e.g., as illustrated in FIGS. 11A and 11B), and further a plurality of amino acid indices (not illustrated in FIG. 11A or 11B), or a low dimension or latent dimension thereof, for each of the naturally occurring amino acids in a third dimension. Such low dimension or latent dimension thereof, for each of the naturally occurring amino acids are disclosed in Nakai et al. 1988, Protein Eng. 2, pp. 93-100, which is hereby incorporated by reference. In some embodiments, the low dimension or latent dimension thereof of the three-dimensional matrix 1100 is a reduced (e.g., factor) for a high-dimensional matrix of data composed of many related variables. However, the present disclosure is not limited thereto. For example, in some embodiments, N-dimensional space includes a three-dimensional encoding based on one or more physicochemical properties 112 of amino acids. In some embodiments, the one or more physicochemical properties 112 of the amino acids include an α-Helicity, a β-sheet propensity, a size, a hydrophobicity, a bulkiness, an isoelectric point, a composition, or a combination thereof. Accordingly, in some such embodiments, the N-dimensional space includes at least three independent indices (e.g., dimensions) that include information relevant to a structure of the target protein 108.

Block 246. Referring to block 246, in some embodiments, the surrogate model 112 is a supervised learning model 116, an unsupervised learning model 116, a temporal difference learning model 116, a reinforcement learning model 116, or the like. For instance, in some such embodiments, the surrogate model 116 is a support vector regression with RBF kernel (SVR-RBF), a random forest (RF), XGBoost, a Gaussian Process (e.g., a collection of random variables indexed by time or space), a deep neural network (DNN), a convolutional neural network (CNN) or a recurrent neural network (RNN). For instance, as a non-limiting example, in some embodiments, the surrogate model 116 is a XGBoost model 116 that includes an XGB Regressor model 116, which is an optimized distributed gradient boosting model 116 that utilizes a scikit-learn estimator when applied to regression problems. As yet another non-limiting example, the CNN surrogate model 116 includes a plurality of convolutional layers that perform various convolution operations between the input values and one or more convolution filters (e.g., N-dimensional space including a matrix of weights) that is learned over many gradient update iterations during the training of the surrogate model 116. Moreover, by utilizing the Gaussian process, the surrogate model 116 provides a prediction for selecting a new data point (e.g., region within the N-dimensional space) using search model, such as by determining a mean fitness and/or uncertainty of a respective point or region within the N-dimensional space. For instance, in some embodiments, given the N-dimensional space, the Gaussian processes surrogate model 116 the method 200 tunes one or more prediction parameters, one or more uncertainty parameters, one or more confidence parameters, or a combination thereof when training in the N-dimensional space.

In some such embodiments, the surrogate model during training outputs an estimated value for the first property of a respective combinatorially substituted protein in the first plurality of combinatorially proteins for each respective combinatorially substituted protein in the first plurality of combinatorially proteins upon input of an encoding, such as matrix 1100 or any of the other encodings disclosed herein for the respective combinatorially substituted protein. The estimated value for the first property assigned by the surrogate mode to each respective combinatorially substituted protein in the first plurality of combinatorially proteins during training is then compared to the corresponding measured values for the first property for each of the combinatorially substituted proteins in the first plurality of combinatorially proteins obtained as described above in block 234. Deviations between actual measured values for the first property and values for the first property calculated by the surrogate model are then back-propagated through the weights of the surrogate model in order to train the surrogate model. For instance, in the case where the surrogate model is a convolutional neural network, the filter weights of respective filters in the convolutional layers of the network are adjusted in such back-propagation. In an exemplary embodiment, the surrogate model is trained against the deviations between actual measured values for the first property and values for the first property calculated by the surrogate model by stochastic gradient descent with the AdaDelta adaptive learning method (Zeiler, 2012 “ADADELTA: an adaptive learning rate method,” CoRR, vol, abs/1212.5701, which is hereby incorporated by reference), and the back propagation algorithm provided in Rumelhart et al., 1988, “Neurocomputing: Foundations of research,” ch. Learning Representations by Back-propagating Errors. pp. 696-699, Cambridge, MA, USA: MIT Press, which is hereby incorporated by reference. In some embodiments, rather than requiring the surrogate model to call an actual scalar value (e.g., as a regressor), the surrogate model is in the form of a classifier with two possible activity classes (e.g., active and inactive) with respect to the first property. Any misclassification of the respective combinatorially substituted protein in the first plurality of combinatorially proteins with respect to measured classifications of such proteins can be used to train the surrogate model using, for example, the back-propagation techniques discuss above.

Regardless of what type of model 116 is used for the surrogate model 116, the surrogate model 116 makes use of each single point mutation in the respective combinatorially substituted protein in order to update a search model 116 by balancing trade-offs between exploration and exploitation of the N-dimensional space.

Block 248. Referring to block 248 of FIG. 2E, the method 200 includes using the surrogate model 116 and the identity of each single point mutation in the respective combinatorially substituted protein for each respective combinatorially substituted protein in the first plurality of combinatorially substituted proteins to update a search model (e.g., second model 116-2 of FIG. 1B). The search model 116 is configured to determine optimal locations (e.g., points) within the N-dimensional space to sample, such as particular single point mutations to sample for inclusion within the one or more combinatorially substituted proteins. Accordingly, the search model 116 is updated to exploit regions of the N-dimensional space where the surrogate model 116 determines optimal objective features (e.g., maximum values for the corresponding measured value of the first property). Moreover, in some embodiments, the search model is updated to explores regions of the N-dimensional space that the surrogate model 116 indicates has a high uncertainty for prediction. However, the present disclosure is not limited thereto.

In some embodiments, the updating the search model 116 includes partitioning the N-dimensional space, such as by forming a M-dimensional sub-space within the N-dimensional space. M is a positive integer less than or equal to N. In some embodiments, this partitioning of the N-dimensional space by the surrogate model 116 forms a first partition that is representative of each single point mutation in the respective combinatorially substituted protein that satisfies a corresponding threshold value requirement for the respective property 112. Said otherwise, each single point mutation of the first partition is a best or worst performing point mutation for affecting the first property of the target protein. In this way, the search model 116 is utilized to further explore the N-dimensional space based on the learned information gained by the surrogate model 116 that is trained in the N-dimensional space. For instance, in some embodiments, the surrogate model 116 is trained in the N-dimensional space to partition the N-dimensional space and a respective partitioning is used by the search model 116 to determine the identity of the one or more combinatorial substitutions that affect the first property 112-1 of the target protein.

By identifying each single point mutation in the respective combinatorially substituted protein for each respective combinatorially substituted protein in the first plurality of combinatorially substituted proteins, the method 200 provides a more discriminative and smaller feature set for using when identifying the one or more combinatorial substitutions that affect the first property 112-1 of the target protein 108. Accordingly, in some such embodiments, by updating the search model 116, the method 200 provides an updated search model 116 based on the optimal feature space within the N-dimensional space identified by the surrogate model 116. As such, the training of the surrogate model 116 transforms the first plurality of combinatorially substituted proteins from an initial state into a state which better identifies a second plurality of combinatorially substituted proteins within the N-dimensional space. From this, when the training of the surrogate model 116 is successful, then the updated search model 116 is applied to the N-dimensional space and a correct or at least reasonable output (e.g., identify a second plurality of combinatorially substituted proteins within the N-dimensional space) is obtained.

Block 250. Referring to block 250, in some embodiments, the method 200 includes using the updated search model 116 to identify a second plurality of combinatorially substituted proteins within the N-dimensional space. By using the updated search model 116 to identify the second plurality of combinatorially substituted proteins within the N-dimensional space, the method 200 greatly reduces the number of evaluations used to explore the N-dimensional since and search for the identity of the one or more combinatorial substitutions that affect the first property 112-1 of the target protein by optimizing the search model 116 in the form of the updated search model 116 provided by the surrogate model 116. In this way, each respective combinatorially substituted protein in the second plurality of combinatorially substituted proteins is characterized by the reference sequence for the target protein 108 with the exception of independent inclusion of two or more single point mutations from the second plurality of single point mutations. As a non-limiting example, in some such embodiments, the using the updated search model 116 identifying an ideal mutation rate range that significantly improves specific protein functions (e.g., the first property 112-1 that affects the target protein 108). In some embodiments, the ideal mutation rate is in a range of from about 1 to 500 mutations per mutant, from about 2 to 100 mutations per mutant, from about 3 to 50 mutations per mutant, from about 5 to about 30 mutations per mutant, or a combination thereof.

Block 252. Referring to block 252, in some embodiments, the use of the updated search model 116 identifies an optimal range of single point mutations, drawn from the second plurality of single point mutations to incorporate into the target protein 108. For instance, in some embodiments, the updated search model 116 identifies one or more clusters of single point mutations that form an optimal range of single point mutations that, when combinatorially substituted, affect the first property of the target protein. However, the present disclosure is not limited thereto. In some embodiments, the updated search model 116 identifies the optimal range of single point mutations by partitioning the N-dimensional space based on structurally similar single point mutations of proteins that share little to no sequence identity, which allows for forming optimal ranges of homologous combinatorially substituted sequences. In some embodiments, the optimal range of single point mutations based on a correlation between the corresponding threshold value requirement for the respective property 112 and empirical data sets associated with the respective property 112.

Block 254. Referring to block 254, in some embodiments, the use of the updated search model 116 identifies one or more optimal single point mutations in the second plurality of single point mutations to incorporate into the target protein 108. In some embodiments, the one or more optimal single point mutations in the second plurality of single point mutations is identified by selecting each single point mutation in the first plurality of single point mutations that is determined to support affecting the first property 112-1 of the target protein. However, the present disclosure is not limited thereto. For instance, in some embodiments, the one or more optimal single point mutations in the second plurality of single point mutations is identified by selecting each single point mutation in the first plurality of single point mutations that is determined to satisfy a corresponding threshold value requirement for a property 112 in the set of properties 112 (e.g., block 228 of FIG. 2C).

Block 256. Referring to block 256, in some embodiments, the use of the updated search model 116 rank orders each single point mutation in the second plurality of single point mutations to incorporate into the target protein 108. By determining the rank order of each single point mutation in the second plurality of single point mutations to incorporate into the target protein 108, the updated search model 116 provides an indication of a hierarchy (e.g., the relative ranks) of the single point mutations in the second plurality of point mutations. As a non-limiting example, in some embodiments, the rank ordering is a full rank ordering, which includes sorting each single point-mutation in the second plurality of single point mutations. As another non-limiting example, in some embodiments, the rank ordering is a partial rank ordering, such as sorting of the extrema values (e.g., finding some largest values and some smallest values in the N-dimensional space). For instance, in some embodiments, the rank ordering select the largest (e.g., positive) values and/or selects the smallest (e.g., negative) values out of the N-dimensional space. However, the present disclosure is not limited thereto. In some embodiments, the rank ordering orders each single point mutation in the second plurality of single point mutations using only the corresponding measured value of the first property 112-1 as a classification feature. By isolating the rank orders based on the first property 112-1, the search model 112 identifies the second plurality of combinatorially substituted proteins within the N-dimensional space by identifying the optimal attributes of parameters of the surrogate model 116.

### EXAMPLE 1. A COMPUTER SYSTEM ARCHITECTURE

A computer system in accordance with the present disclosure (e.g., protein library 106 of FIG. 1A, model library 114 of FIG. 1B) was designed to combine single mutations that were determined to be beneficial to neutral using an in silico detection method (e.g., method 200 of FIGS. 2A through 2E).

The method 200 used a plurality of models 116 such as or more molecular dynamics free energy simulations models 116, one or more atomistic models 116, one or more machine learning/deep learning models 116, which was utilized to search for beneficial to neutral mutations of a target protein (e.g., protein T 108-T of FIG. 1A).

Specifically, in silico calculation of one or more properties 112 described herein (e.g., first property 112-1, second property 112-2, . . . , property P 112-P of FIG. 1A) included determining a mutation energy (e.g., stability property 112 of the target protein 108), determining a mutation energy (e.g., binding property 112 of the target protein 108), selecting one or more non-conserved positions and observed mutations in one or more homologs of the target protein 108, determining one or more formulation properties 112 of the target protein 108, evaluating an estimate of a post-translational modification of the target protein 108, evaluating an immunogenicity property 112 of the target protein 108, or a combination thereof.

### EXAMPLE 1.1. DETERMINING A MUTATIONAL ENERGY (STABILITY PROPERTY) OF NEOCALLIMASTIX PATRICIARUM XYLANASE

A mutation energy (e.g., stability property 112 of the target protein 108) was determined in order to evaluate an effect of one or more combinatorial substitutions (e.g., mutations) on the stability of the target protein 108.

In some embodiments, the mutation energy (e.g., stability property 112 of the target protein 108) was determined for 60 positions of a xylanase from Neocallimastix patriciarum at low pH (e.g., first plurality of single point 110 mutations, block 216 of FIG. 2B through block 232 of FIG. 2C or a combination thereof, etc.). The 60 selected positions were mutated to 19 other amino acids respectively, resulting in 1140 single point mutants of the target protein 108 based on the various combinations for the 19 other amino acids at the 60 selected positions. Three crystal structures reported for this enzyme of 3WP4, 3WP5, and 3WP6 were used for the determination of the mutation energy (e.g., stability property 112 of the target protein 108). The results of such determinations are shown in FIG. 3. More specifically, FIG. 3 illustrates a user interface 300-1 that presents a chart of the results that included the determination of the mutation energy (stability) for Beneficial-Neutral (B-N) and Deleterious (D) groups of 1140 single mutants of Neocallimastix patriciarum xylanase, in which B-N or D was determined experimentally at pH 2.5, 37° ° C., comparing to a wild type enzyme. In FIG. 3, the Y-axis is the determination of the mutation energy (stability) for the 1140 point 110 mutations based on the three crystal structures alone and an average (“AVG”) that was the average mutation energy (e.g., stability property 112 of the target protein 108) of the three crystal structures. To validate the accuracy of the determinations of the mutation energy, various experiments were conducted to evaluate an activity of the same set of 1140 single sequence point 110 mutants (e.g., repeating block 216 of FIG. 2B for an identical first plurality of single point mutations of the target protein 108). Sequence point 110 mutants that performed equally or better than the wild type sequence points 110 were classified as Beneficial-Neutral (B-N), and all other sequence point 110 mutants were classified as Deleterious (D). As shown in FIG. 3, about 20% sequence point 110 mutations with very positive mutation energy are indeed deleterious sequence point 110 mutations. This result verified the utilization of the determination of the mutation energy to eliminate very deleterious sequence point 110 mutations (e.g., block 228 of FIG. 2C).

One of skill in the art in view of the present disclosure will appreciate that the 60 positions evaluated in this example are mostly located on a surface of the target protein 108. Accordingly, some or all of the 60 positions are exposed to solvent, where the determination of the mutation energy is known to be more challenging in comparison to buried positions (e.g., not exposed to solvent). Moreover, in some embodiments, the results presented in FIG. 3 were more effective when applied to the whole target protein 108 including both surface and buried positions.

### EXAMPLE 1.2. DESIGNING AND EVALUATING INTELLIGENT LIBRARIES OF PULLULANASE

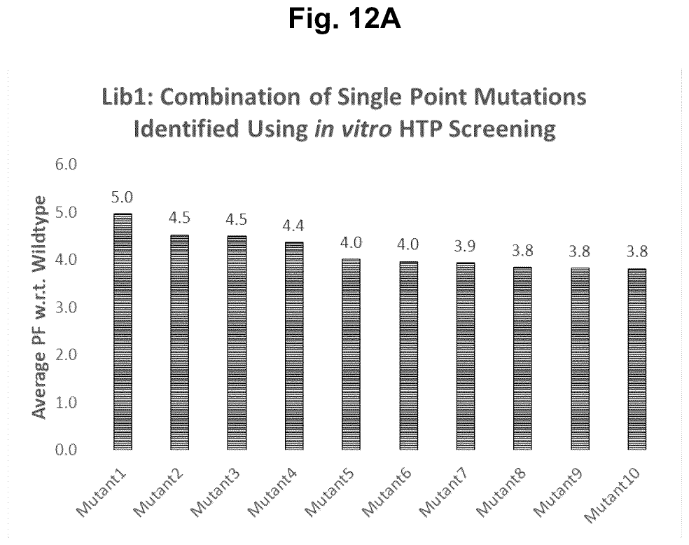

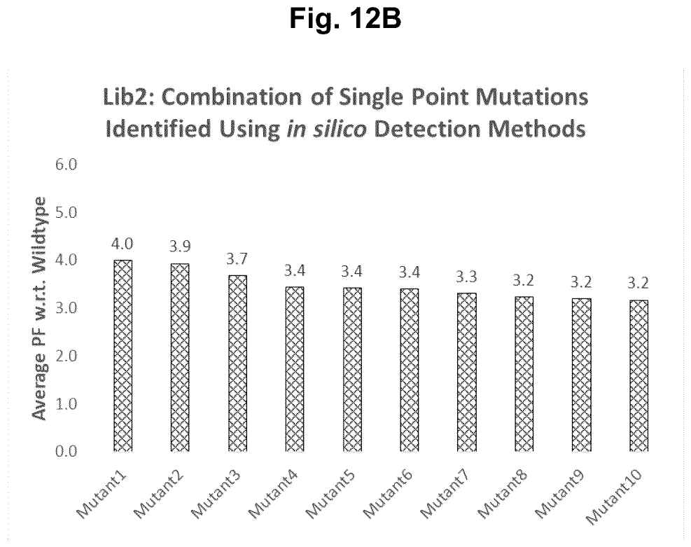

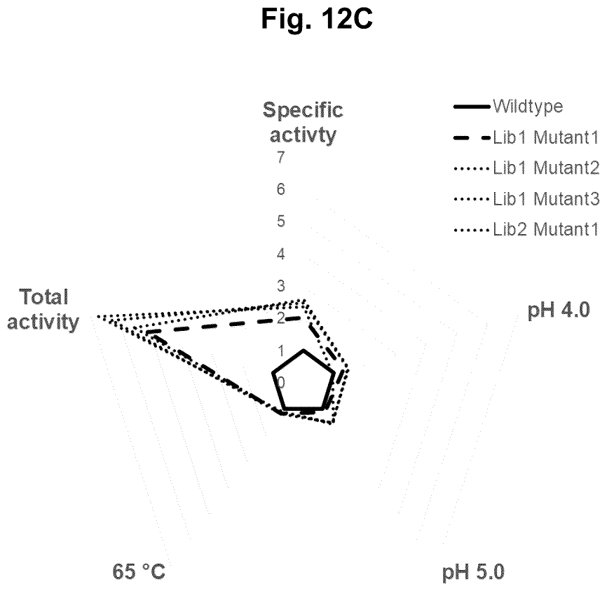

To improve the specific activity of a pullulanase of interest, two intelligent libraries were designed and constructed using proprietary technology. Lib1 was designed to combine 74 single point mutations that were determined to be Beneficial-Neutral using a range of in silico detection methods including but not limited to the methods described herein. Lib2 was designed to combine 71 single point mutations that were determined to be Beneficial-Neutral using in vitro high throughput screening for activity on pullulan substrate at pH 4.5 and 60° ° C., which is the optimal pH and temperature condition for this pullulanase. Notably there are only 5 single point mutations in common between Lib1 and Lib2. Thousands of mutants from each library were screened for activity on pullulan substrate at pH 4.5 and 60° ° C. FIGS. 12A and 12B show the performance factor of top 10 mutants identified from these two libraries respectively and the top mutants from Lib1 significantly outperformed the top mutants from Lib2.

3 Top mutants from Lib1 and 1 top mutant from Lib2 were further characterized at different conditions. As shown in FIG. 12C, the top mutants from Lib1 have significantly improved properties including not only specific activity, but also total activity and low pH activity without compromising high temperature stability compared to the parent pullulanase. The top mutant from Lib2 is also improved compared to the parent pullulanase however not as good as the top mutants from Lib1.

Multi-parameter optimization of proteins may be a challenge. Herein, using a wide range of in silico detection methods, different properties of single point mutations can be assessed to remove deleterious mutations. As a result, mutants identified from the library that combines potentially Beneficial-Neutral single mutations can provide a balanced solution. In silico-based design may be more cost effective and less time consuming than in vitro screening based design where effective HTP screening strategies at different conditions need to be established and executed.

### EXAMPLE 2. DETERMINATION OF THE RELATIONSHIP BETWEEN MUTATION RATE AND FUNCTION AND EVALUATION OF INTERACTIONS BETWEEN SINGLE POINT MUTATIONS

An endoglucase from Aspergillus udagawae (Accession Number A0A0K8LET0) was chosen as a model system. Twenty-two pre-selected single sequence point 110 mutants were evaluated using a colorimetric assay that measures activity on CarboxyMethylCellulose (CMC) at a pH of about 6.5, a temperature of 50 degrees Celsius (° C.) for about three hours. The results from this evaluation are shown in FIG. 4. A performance factor (PF) was determined based on a ratio of mutant OD reading and wild type OD reading. The 22 single point 110 mutations are split into a beneficial (B-Muts) group, a neutral (N-Muts) group, a deleterious (D-Muts) group, or a combination thereof based on their performance factor. In some embodiments, the B-Muts group included sequence point 110 mutations with PF value ≥1.2, (e.g., G142T, N28E, G58S, Q144R and D104N of FIG. 4). In some embodiments, the N-Muts group included sequence point 110 mutations with a PF value greater than 0.8 and less than 1.2 (e.g., G41S, H198T, T64N, R221P, A60D, S55Y, T223E, T181Y, T85D. S182V, S102Q, D189L, D7IP, R192Q and K87S of FIG. 4). Moreover, in some embodiments, the D-Muts group included sequence point 110 mutations with a PF value of less than or equal to 0.8 (e.g., S227F and N197S of FIG. 4). However, the present disclosure is not limited thereto.

The 22 point mutations were collectively considered as a second plurality of single point 110 mutations in order to form a protein library 106 comprising a first plurality of combinatorially substituted proteins (e.g., block 234 of FIG. 2D). About 600-700 combinatorially substituted proteins were evaluated using a HTP CMC assay (N=1). Moreover, about 60-70 combinatorially substituted proteins with a broad range of CMC activity were selected for sequencing and CMC activity retesting (N=3). FIG. 5 illustrates a chart 500 of a relationship between a mutation rate (e.g., number of mutations per mutant) from mutant sequencing and average PF from CMC activity resting. Here, the standard deviation for average PF was 0.04±0.03. The combinatorially substituted proteins has 3-10 mutations per mutant and the PF ranges between 0.5-2.3. The top performing combinatorially substituted proteins with PF 2.3 have 7-10 mutations (e.g., FIGS. 6A through 6D).

To understand epistatic interactions between these 22 point mutations, all 231 possible pairwise mutants from the 22 point mutations were constructed and evaluated for CMC activity. The absolute epistatic deviation AED was determined based on as PF_{Mut1/Mut2}−PF_{Mut1}×PF_{Mut2}. A positive AED value provided positive epistatic interactions between two mutations, whereas a negative AED value provided negative epistatic interactions. From this, an interaction network was derived, with a majority of the epistatic interactions being positive. Referring briefly to FIGS. 6A through 6D, an interaction network for the top mutants with PF value of 2.3 were evaluated.

Referring to FIG. 6A, N28E/G41S/A60D/D71P/K87S/D104N/G142T/R192Q/R221P/T223E includes 3 B-Muts (N28E, D104N and G142T), 7 N-Muts, 0 D-Muts, 7 negative epistatic interactions, and 38 positive epistatic interactions. All the negative epistatic interactions [N28E/(D104N or R221P), D104N/(G142T, T223E or R221P), G142T/(G41S or R221P)] included B-Muts. In contrast, all the positive epistatic interactions included N-Muts.

Referring to FIG. 6B, N28E/G41S/A60D/T64N/D71P/G142T/R192Q/R221P includes 2 B-Muts (N28E and G142T), 6 N-Muts, 0 D-Muts, 4 negative epistatic interactions, and 24 positive epistatic interactions. All the negative epistatic interactions [N28E/(T64N or R221P), G142T/(G41S or R221P)] included B-Muts. In contrast, all the positive epistatic interactions included N-Muts.

Referring to FIG. 6C, N28E/G41S/G58S/T64N/T85D/G142T/T181Y/R221P includes 3 B-Muts (N28E, G58S and G142T), 5 N-Muts, 0 D-Muts, 8 negative epistatic interactions, and 20 positive epistatic interactions. All the negative interactions [N28E/(T64N, G58S or R221P), G58S/G142T, G142T/(G41S, R221P or T181Y) except for [G41S/T181Y] included B-Muts. In contrast, all the positive epistatic interactions included N-Muts.

Referring to FIG. 6D, N28E/G41S/S55Y/D71P/D104N/G142T/R221P included 2 B-Muts (N28E, D104N and G142T), 5 N-Muts, 0 D-Muts, 8 negative epistatic interactions, and 13 positive epistatic interactions. All the negative interactions [N28E/(D104N or R221P), D104N/(G142T or R221P), G142T/(G41S or R221P)] except for [G41S/S55Y, S55Y/R221P] includes B-Muts. In contrast, all the positive epistatic interactions included N-Muts.

In some embodiments, B-Muts, although beneficial by themselves, often lead to negative epistatic interactions when combined, which led to a rarity when identifying point mutants that contain B-Muts only. On the other hand, N-Muts, although neutral by themselves, generally lead to positive epistatic interactions. In some embodiments, D-Muts were avoided since the deleterious effects provided by the D-Muts could not readily be offset by positive epistatic interactions. Accordingly, in some such embodiments, an ideal combination was, therefore, between B-Muts and N-Muts. Although there was a limited number of B-Muts in the target protein 108, there was usually a much larger pool of N-Muts (e.g., by a factor of 1, a factor of 2, a factor of 5, a factor of 10, a factor of 100, etc.). Accordingly, the systems and methods of the present disclosure provided a protein library 106 that combined hundreds of B-Muts and N-Muts.

### EXAMPLE 3. CONSTRUCTION AND SCREENING OF PROTEIN LIBRARIES INCLUDING COMBINATORIALLY SUBSTITUTED PROTEINS

Conventionally, a protein library 106 combines multiple mutations that are made with well-known combinatorial library generation methods. However, such an approach can only target a very limited number of mutations and regions of the target protein 108. Therefore, conventional library construction approaches limit the opportunity to include N-Muts and activate positive epistatic interactions. Consequently, under convention approaches, iterative rounds of combinatorically substituted proteins are required and the search for the best performing mutants is inefficient, costly, and path dependent.

To resolve this challenge, the systems and methods of the present disclosure constructed the computer system 100 including the protein library 106 and the model library 114. The computer system 100 provided several key advantages and characteristics including obtaining an identity of each single point 110 mutation in a first plurality of single point mutations of the target protein 108 includes hundreds of carefully selected single point 110 mutations from Example 1 that occur at N positions of the target protein 108, each mutant contains 1-N mutations, the mutations could occur both at different positions and/or at the same positions, and mutating positions can be either far away from each or very close-by on sequence or structure. Accordingly, in some such embodiments, from the second plurality of single point mutations, the systems and methods of the present disclosure form a first plurality of combinatorially substituted proteins that is prepared for HTP screening and sequencing.

### EXAMPLE 3.1. CONSTRUCTION. SCREENING AND SEQUENCING OF PROTEIN LIBRARIES INCLUDING COMBINATORIALLY SUBSTITUTED PROTEINS OF ASPERGILLUS UDAGAWAE ENDOGLUCANASE

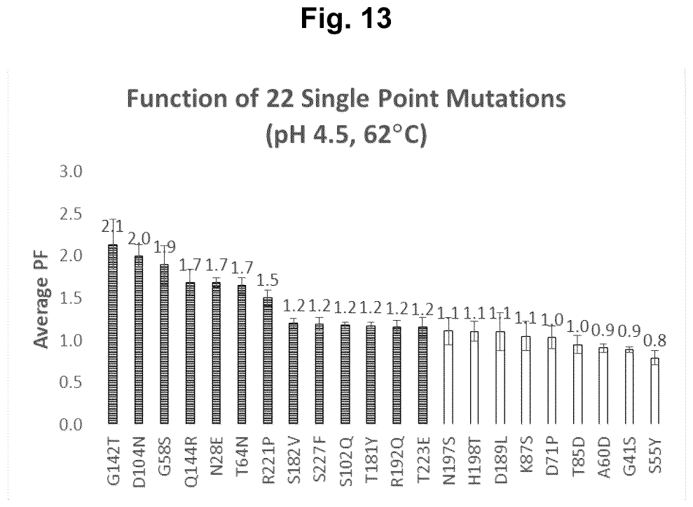

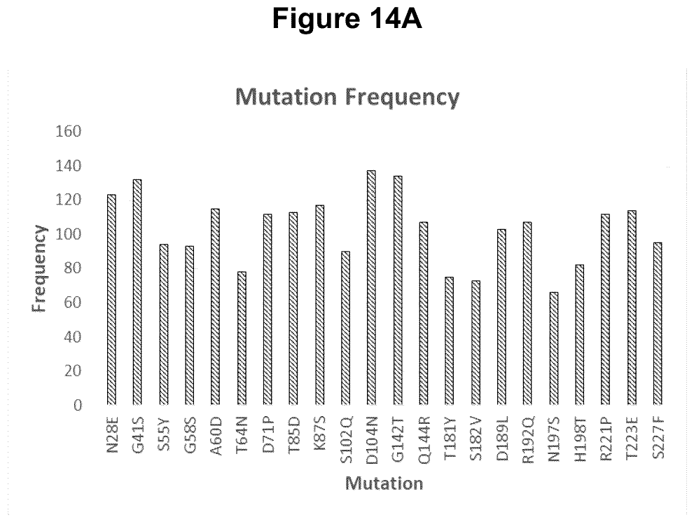

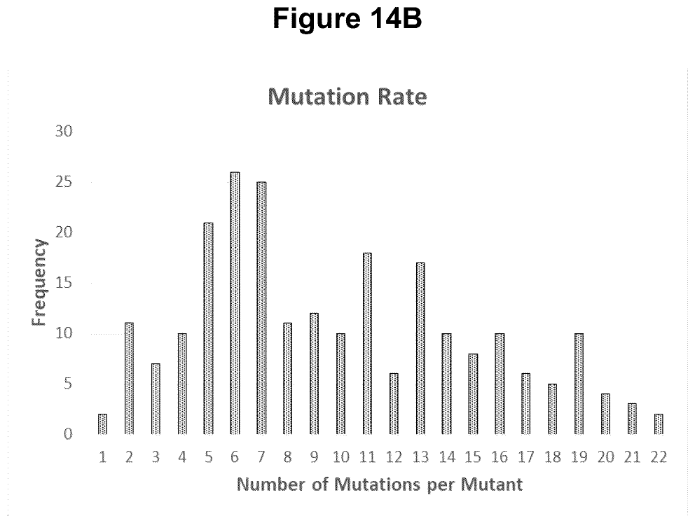

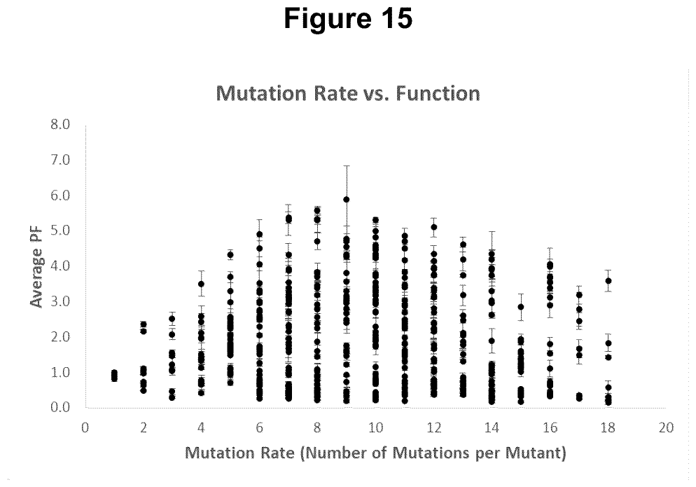

The 22 single point mutations in Example 2 were evaluated for CMC activity at a pH of about 4.5, a temperature of 62 degrees Celsius (° C.) for about three hours. As shown in FIG. 13, B-Muts include G142T, D104N, G58S, Q144R, N28E, T64N, R221P, S182V, S227F, S102Q, T181Y, R192Q and T223E: N-Muts include N197S, H198T, D189L, K87S, D71P, T85D, A60D, G41S and S55Y: no D-Muts. A protein library of Aspergillus udagawae endoglucanase comprising these 22 Beneficial-Neutral mutations was constructed using proprietary technology. A random set of mutants were drawn from this library to assess its quality in terms of mutation frequency and mutation rate. As shown in FIGS. 14A and 14B, the protein library can sample all 22 mutations and 1-22 mutations per mutant, which is desirable for studying interactions between mutations in Aspergillus udagawae endoglucanase-like proteins. Over 3000 mutants from a protein library comprising these 22 mutations were evaluated using a HTP CMC assay (N=1). Moreover, about 400 combinatorially substituted proteins with a broad range of CMC activity were selected for sequencing and CMC activity retesting (N=3). FIG. 15 illustrates a chart of the relationship between mutation rate (e.g., number of mutations per mutant) from mutant sequencing and average PF from CMC activity retesting. Here, the standard deviation for average PF was 0.16±0.14. The combinatorially substituted proteins have 1-18 mutations per mutant and the PF ranges between 0.1-5.9. The best performing combinatorially substituted protein with the PF 5.9 has 9 mutations.

### EXAMPLE 3.2. CONSTRUCTION. SCREENING AND SEQUENCING OF PROTEIN LIBRARIES INCLUDING COMBINATORIALLY SUBSTITUTED PROTEINS OF CHAETOMIUM THERMOPHILUM ENDOGLUCANASE

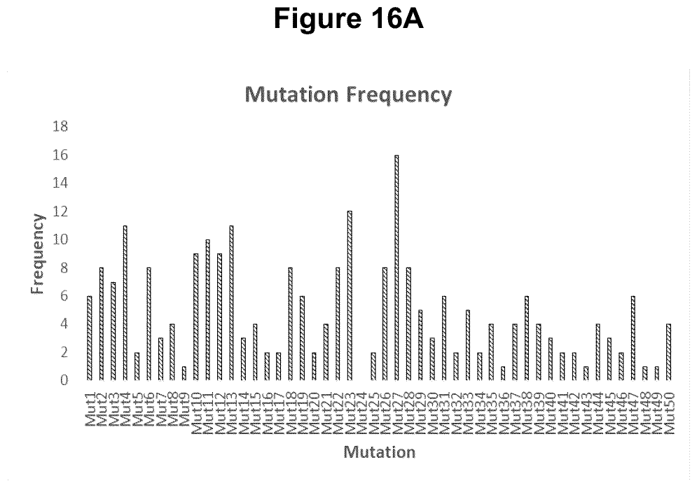

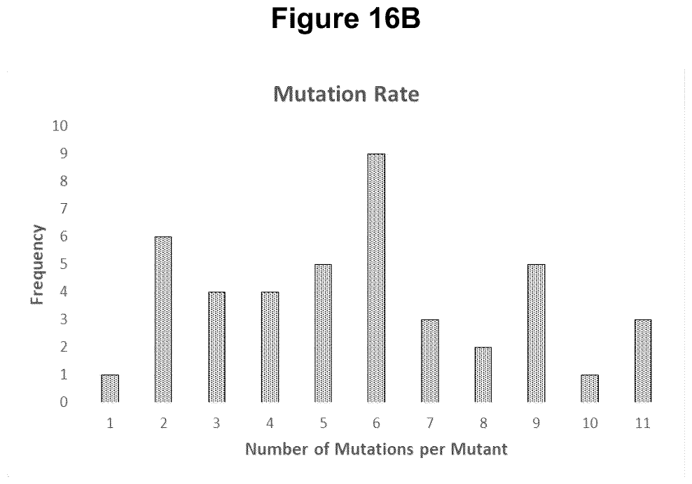

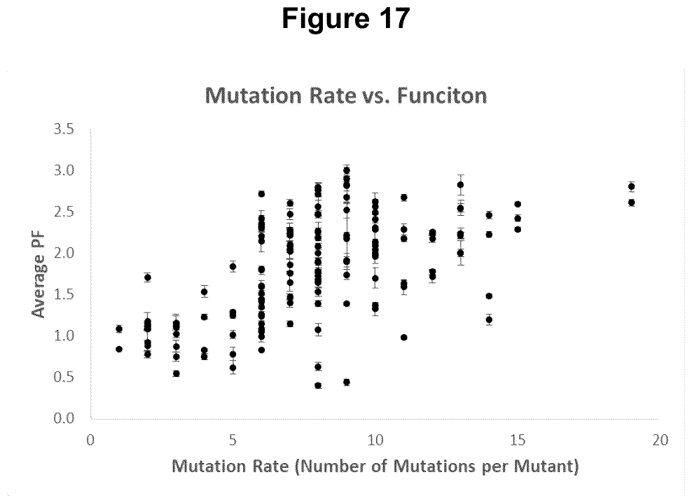

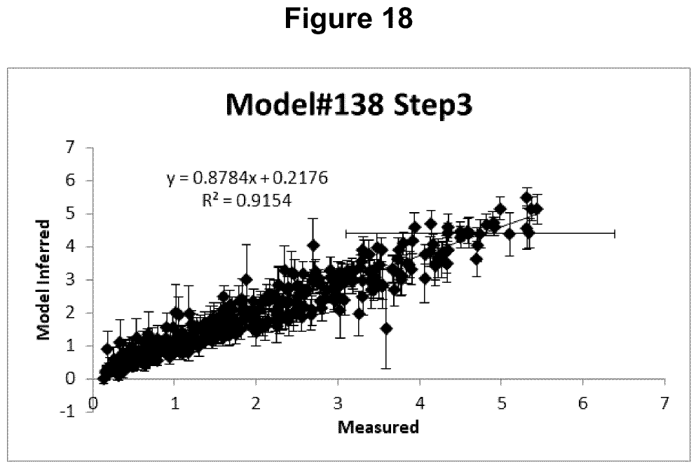

The protein library of Chaetomium thermophilum endoglucanase comprising 50 Beneficial-Neutral mutations identified from in vitro HTP screening of site saturation mutagenesis libraries using a colorimetric assay that measures activity on Carboxy MethylCellulose (CMC) at a pH of about 6.5, a temperature of 50 degrees Celsius (° C.) for about three hours. The protein library was constructed using proprietary technology. A random set of mutants were draw from this library to assess its quality in terms of mutation frequency and mutation rate. As shown in FIGS. 16A and 16B, the protein library can sample majority of the 50 mutations and 1-11 mutations per mutant. Over 2000 combinatorially substituted protein from this library were evaluated using the HTP CMC assay (N=1). Subsequently, about 150 combinatorially substituted proteins with a broad range of activity were selected for sequencing and CMC activity retesting (N=3). FIG. 18 illustrates a relationship between mutation rate and average PF. Here, the standard deviation for average PF was 0.06±0.04. The combinatorially substituted proteins has 1-19 mutations per mutant and the PF ranges between 0.4-3.0. The top performing combinatorially substituted protein with PF 3.0 has 9 mutations. For Chaetomium thermophilum endoglucanase-like proteins which can tolerate more than 11 mutations per mutants, mutants with higher mutation rate indeed exist in the library and can be enriched by HTP screening.

### EXAMPLE 4. ENCODING A DATA SET AND TRAINING A SURROGATE MODEL

In some embodiments, after constructing, HTP screening, and sequencing a first plurality of combinatorially substituted proteins (e.g., block 234 of FIG. 2D), the mutation and function (e.g., PF) results were compiled for use with a plurality of models 116, which included a surrogate model 116 (e.g., first model 116-1 of FIG. 1B) and a search model 116 (e.g., second model 116-2 of FIG. 1B). Accordingly, an N-dimensional space (e.g., block 240 of FIG. 2D) was formed based on mutations as a data set and function (e.g., PF) results as labels. Accordingly, in some embodiments, the mutation data set was initially encoded into numerical encodings that were suitable for input for training a respective model 112. Referring briefly to FIG. 7A and FIGS. 11A and 11B, various examples of encodings are provided.

More particularly, referring briefly to FIG. 11A, in some embodiments, the N-dimensional space included a 2-dimensional mutation matrix. In some embodiments, in the 2-dimensional mutation matrix, one axis represents a sequence position, and the other axis represents mutants (e.g., in which a 1 at a particular residue type on the other axis represents a point 110 mutation of the residue type is present and 0 indicates absence of a mutation.

In some embodiments, the N-dimensional space included a 3-dimensional one-hot encoding (e.g., one-of-K scheme for encoding by converting categorical variables). In some embodiments, the 3-dimensional encoding included an X-axis that represents amino acid positions (sequence position), a Y-axis that represents absence or presence of mutants and their identity, and a Z-axis that represents addition information about amino acids. Accordingly, presence of a “1” represents an amino acid that is present and “0” represents an amino acid that is absent at a particular position, in some encoding embodiments.

In some embodiments, the N-dimensional space included a 3-dimensional encoding based on physicochemical parameters of amino acids. In some embodiments, in this 3-dimensional encoding, one axis represented amino acid positions (sequence position), another axis represented mutants, and a Z-axis represented 19 low-dimension representations of over 500 amino acid indices from the amino acid index (AAIndex) database (e.g., a set of uncorrelated scales satisfying a varimax criterion). See Georgiv, A., 2009, “Interpretable Numerical Descriptors of Amino Acid Space.” Journal of Computational Biology, 16(5), pg. 703-723, which is hereby incorporated by reference in its entirety.

In some embodiments, the N-dimensional space included a 3-dimensional embedding generated by one or more fully unsupervised models 116, such as a model 116 based on a dilated residual network architecture (e.g., ResNet), Transformer based on a transformer architecture, Bepler, UniRep and LSTM based on LSTM architectures. See. Rao et al., 2019, “Evaluating Protein Transfer Learning with TAPE,” Advances in Neural Information Processing Systems, 32, pg. 9689: Chang et al., 2017, “Dilated Recurrent Neural Networks,” arXiv preprint arXiv: 1710.02224; Vaswani et al., “Attention Is All You Need,” Advances in Neural Information Processing Systems,” pg. 5998-6008: Bepler et al., 2019, “Learning Protein Sequence Embeddings Using Information from Structures,” arXiv preprint arXiv: 1902.08661: Alley et al., 2019, “Unified Rational Protein Engineering with Sequence-based Deep Representation Learning,” Nature Methods, 16(12), pg. 1315-1322: Schmidhuber et al., 1997, “Long Short-term Memory,” Neural Comput, 9(8), pg. 1735-1780, each of which is hereby incorporated by reference in its entirety. In 3-D embeddings, one was for amino acid positions, another axis was for mutant identity, and a third axis was for latent dimensions. See, for example, Bepler 100, ResNet 256, Transformer 512, UniRep 1900, which is hereby incorporated by reference.

Referring briefly to FIGS. 7B through 7F, a process (e.g., block 240 of FIG. 2D) of an automated workflow for training a surrogate model 116 was provided.

In some embodiments, referring to FIG. 7B, initially, a suitable surrogate model 116 was determined and tuned (e.g., hyperparameter tuning of the surrogate model 116). A plurality of non-linear regularized surrogate models 116 was tested. For instance, in some embodiments, the surrogate model 116 tested was a support vector regression with rational basis function (RBF) kernel (SVR-RBF) model 116, a random forest (RF) model 116, an XGBoost boost model 116, a gaussian process model 116, a deep neural network (DNN) 116, a convolutional neural network (CNN) 116, a recurrent neural network (RNN) 116, or a combination thereof. In some embodiments, the hyperparameter tunning was performed utilizing grid search for machine learning methods and Bayesian optimization (BO) for deep learning methods. In some embodiments, the data set was split into a first training data set, a second validation data set, and a third testing data set. In some embodiments, a respective surrogate model 116 was selected to minimize the mean square error (MSE) for the validation set. In some embodiments, data splitting was repeated multiple times (e.g., twice, three times, five times, ten times, fifty times, a hundred times, a thousand times, etc.) to evaluate how generalizable the respective surrogate model 116 performed.

In some embodiments, referring to FIGS. 7C through 7E, further examination of the model quality of all or any selected models from FIG. 7B was considered. Correlation plots between model predicted function and measured function were visualizable for training, validation and testing data sets in each data splitting.

In some embodiments, referring to FIG. 7F, bootstrapping, model quality/variation, evaluation, and inference of any untested mutants of interest using the best models 116 from FIGS. 7C through 7D was conducted. Since the protein library 106 of the present disclosure allows the combination of many more mutations than conventional libraries, the data from the protein library 106 tends to have a much broader dynamic range and is more effective for the learning process. An example is shown in FIGS. 8A through 8D.

### EXAMPLE 4.1. ENCODING A DATA SET AND TRAINING A SURROGATE MODEL FOR THE PROTEIN LIBRARY OF ASPERGILLUS UDAGAWAE ENDOGLUCANASE

The data set obtained from Example 3.1 was encoded and learnt using various machine learning methods such as SVR, CNN and RNN. FIG. 18 shows one of the step 3 models obtained using CNN (R²=0.92). The data offered by the protein library with rich interactions between all 22 mutations in conjunction with various machine learning and deep learning methods led to surrogate models with decent qualities.

### EXAMPLE 4.2. ENCODING A DATA SET AND TRAINING A SURROGATE MODEL FOR THE PROTEIN LIBRARY OF CHAETOMIUM THERMOPHILUM ENDOGLUCANASE

A library of Chaetomium thermophilum endoglucanase with 50 mutations described in Example 3.2 (first plurality of single point mutations) led to significantly improved mutants (measured PFₘₐₓ=3) and model quality (R²=0.89) (FIG. 8A) than the conventional route where the 50 mutations were split into three conventional libraries (measured PFₘₐₓ=1.7/1.5/2.2, model quality R²=0.55/0.65/0.62) (FIGS. 8B-8D). The modeling method used in this example was SVR-RBF, a simple machine learning method. This example demonstrates quality data is the key for obtaining an accurate predictive model. In some embodiments, the data offered by the protein library 106 with rich interactions between a wide range of mutations led to powerful surrogate models 116 even with simple learning methodologies.

### EXAMPLE 5. OPTIMAL N-DIMENSIONAL SPACE DESIGN AND OPTIMAL MUTANT INFERENCE

Once a reasonable predictive model 116 was established as shown in Example 4, the next challenge was to search a N-dimensional space in order to identify the optimal mutation combinations (e.g., a second plurality of combinatorially substituted proteins within the N-dimensional space, block 250 through block 256 of FIG. 2E, etc.). In some embodiments, such as those that include hundreds of point 110 mutations, the N-dimensional space (e.g., the combinatorial mutation space) is very large. Accordingly, the systems and methods of the present disclosure utilized a search model 116 (e.g., second model 116-2 of FIG. 1B) that is particularly configured to effectively accomplish this task. Accordingly, in some such embodiments, the search model 116 utilized Bayesian optimization to identify combinatorially substituted proteins.

Bayesian optimization is a sequential design strategy for optimization of functions that are expensive to evaluate. In some embodiments, the Bayesian optimization is max_{x∈A}f(x), where f(x) is a difficult-to-evaluate black box function and A is a set of points whose membership can easily be evaluated. The Bayesian model 116 places a prior over the objective function f(x). After gathering one or more initial function evaluations, the prior is updated to form a posterior distribution over the objective function f(x). The posterior distribution is in turn used to construct an acquisition function that determines a next (e.g., subsequent) sampling point 110 within the N-dimensional space. In some embodiments, the Bayesian optimization model 116 is utilized for the tuning of hyperparameters of the search model 116. See Dewancker et al., 2016, “A Stratified Analysis of Bayesian Optimization Methods,” arXiv preprint arXiv: 1603.09441, which is hereby incorporated by reference in its entirety. Accordingly, in the systems and methods of the present disclosure, the Bayesian optimization was applied for tuning the mutations to combine, such that f(x) is the function obtained from Example 4 (e.g., to predict protein function from any given mutation combination) and A is a set of mutations existing in the protein library 106, and the goal of Bayesian optimization was to search for combinatorial mutations that maximize the protein function.

Accordingly, the use of the Bayesian optimization model 116 by the systems and methods of the present disclosure infers optimal mutants to evaluate experimentally, while also providing an optimal plurality of combinatorially substituted proteins to evaluate experimentally.

### EXAMPLE 5.1. OPTIMAL N-DIMENSIONAL SPACE DESIGN FOR ASPERGILLUS UDAGAWAE ENDOGLUCANASE AND EXPERIMENTAL VALIDATION

Referring briefly to Example 2, the 22 point 110 mutations were used for the Bayesian optimization model 116. In some embodiments, the systems and methods of the present disclosure utilized at least three stages of Bayesian optimization including a BO-input stage includes evaluating the mutants identified from the protein library (e.g., block 248 of FIG. 2E), a BO-random stage includes randomly sampling the N-dimensional space, and a BO-opt stage includes searching for mutants (e.g., block 250 of FIG. 2E) that optimize PF of a target protein 108. The results of this approach are shown in FIGS. 9A through 9E. More particularly, FIG. 9A illustrates a good quality of the surrogate model 116 obtained from learning within the N-dimensional space using SVR-RBF (R²=0.85). FIGS. 9C and 9D illustrates steps of the method 200, whereby at the BO-input stage the PF ranges between 0.6-2.3 and mutation rate ranges between 1-10, at the BO-random stage the PF ranges between 0-2.2 and mutation rate ranges between 1-22, and at the BO-opt stage the PF ranges between 2.2-2.9 and mutation rate ranges between 7-17. FIG. 9B illustrates a correlation between the mutation rate and the PF. FIG. 9E illustrates the mutation frequency (e.g., in the protein library 108 and BO-opt). Accordingly, these results collectively demonstrate that an optimal mutation rate identified by Bayesian optimization is 7-17. Moreover, these results collectively demonstrate that optimal mutations to combine identified by Bayesian optimization are R221P, N28E, G142T, D71P, G58S, Q144R, D104N, G41S, S182V, T223E, S102Q, T181Y, T85D, T64N and R192Q, in which weights are from high to low in this order. One of skill in the art of the present disclosure will appreciate that among the top 4 most preferred mutations by Bayesian optimization model 116, R221P and D71P, are neutral mutations by themselves (see FIG. 4), while deleterious mutations, N197S and S227F, are completed rejected by Bayesian optimization. Moreover, because the weights of the identified mutations were provided with a high degree of accuracy, precision, and certainty, the systems and methods of the present disclosure do not require reiterative processes of further updating the search model 116 and/or the surrogate model 116, which greatly improves efficiency.

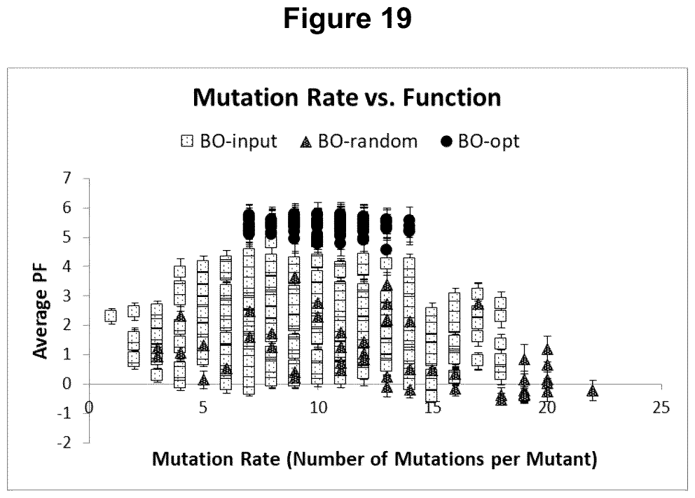

Referring briefly to Example 4.1, the surrogate models derived from the protein library comprising 22 mutations evaluated at pH 4.5, 62° C. were used for the Bayesian optimization search model. As shown in FIG. 19, Bayesian optimization first inferred the PF for about 400 combinatorially substituted proteins in the Example 4.1 data set (PF AVG=0-4.8, mutation rate=1-18 mutations per mutant), next randomly sampled 50 new combinatorially substituted proteins with a broad range of PF and mutation rates (PF AVG=0-3.6, mutation rate=3-22 mutations per mutant), then generated 90 new combinatorially substituted proteins with optimal PF and mutation rate (AVG=4.5-5.8, mutation rate=7-14 mutations per mutant). In addition, 15 mutations occur more frequently in the optimal combinatorially substituted proteins, including N28E, G41S, G58S, D71P, T85D, S102Q, D104N, G142T, Q144R, T181Y, S182V, R192Q, R221P, T223E, S227F indicating they are more preferred for combinations.

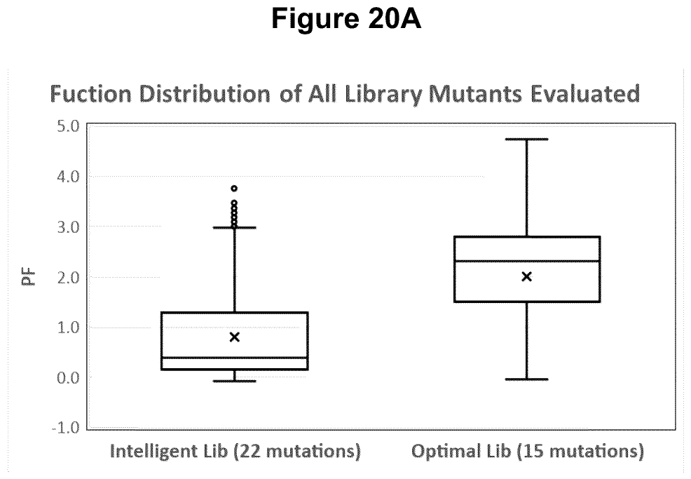

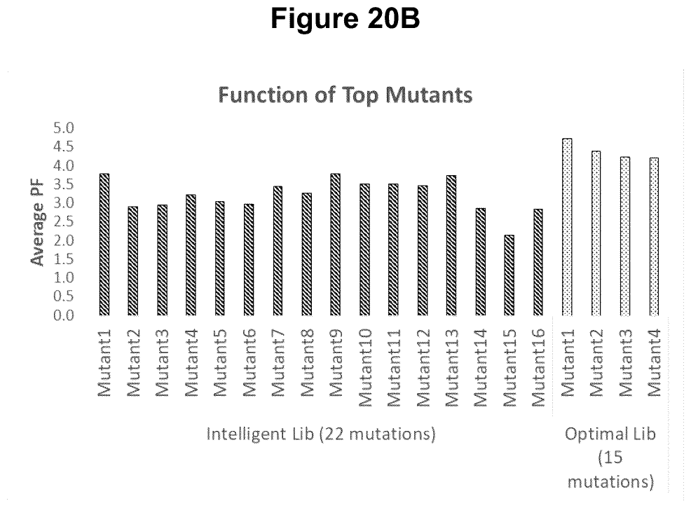

Hence, an optimal library with the 15 most preferred mutations were constructed using proprietary technology and evaluated by the HTP CMC assay. FIG. 20A shows the PF distribution of all mutants (N=1) in the optimal library (15 mutations) and the original intelligent library (22 mutations). FIG. 20B shows the average PF of the top mutants from the intelligent Lib and the optimal Lib (N=6). In both scenarios, the optimal Lib significantly outperforms the intelligent Lib.

### EXAMPLE 5.2. OPTIMAL MUTANT INFERENCE FOR CHAETOMIUM THERMOPHILUM ENDOGLUCANASE AND EXPERIMENTAL VALIDATION

Referring briefly to Example 4.2, the surrogate model 116 developed based on Chaetomium thermophilum Endoglucanase library (e.g., FIGS. 8A through 8D) was used. Six mutants of Chaetomium thermophilum Endoglucanase were inferred by Bayesian optimization to have the highest, medium and low PF. The validation results are shown in FIG. 10A, where a great correlation is observed between measured PF and predicted PF (R²=0.89). Furthermore, the top mutant from Bayesian optimization with PF 3.9 outperforms the top mutants identified from the protein library 106 and conventional library (FIG. 10B).

### EXAMPLE 6. AI-DIRECTED PULLULANASE EVOLUTION

In this example, the following innovative steps improved the desired property of a pullulanase of interest:
Step 1) intelligent library design using various in silico detection methods to measure specifics of single mutations and rule out single deleterious mutations.
Step 2) intelligent library construction that makes mutants with a wide range of mutation rates and allows the deep interactions of different mutations.
Step 3) library screening that generates a diverse set of functional data for the mutants.

Results from Step 1), 2), and 3) for pullulanase 71-Mut Intelligent Lib1 have been discussed in Example 1.2.
Step 4) library mutation-function results that encode an effective learning to acquire a surrogate model without over-fitting. The data set obtained from Step 3) was encoded and learnt using various machine learning methods such as SVR, CNN and RNN. FIG. 21A shows one of the step 3 models obtained using CNN (R²=0.91). The data offered by Intelligent Lib1 with rich interactions between all 71 mutations in conjunction with various machine learning and deep learning methods led to surrogate models with decent qualities.
Step 5) optimal library design using a search model to guide the selection of single residue mutations and mutation rate range. The SVR, CNN and RNN surrogate models derived from Step 4) were used for the Bayesian optimization model. To guide the Bayesian optimization, 35 mutations with the highest PF inferred by the surrogate models were allowed for recombination. As show in FIG. 21B, Bayesian optimization first inferred the PF for 10 best performing combinatorially substituted proteins (PF AVG=1.9-2.4, mutation rate=4-20 mutations per mutant) identified in Step 4), then randomly sampled 50 new combinatorially substituted proteins with a broad range of PF and mutation rate (PF AVG=1.0-3.3, mutation rate=1-33 mutations per mutant), which generated 151 new combinatorially substituted proteins with optimal PF and mutation rate (PF AVG=3.1-3.6, mutation rate=24-35 mutations per mutant).
Step 6) construction and screening of the optimal library to obtain the protein candidates with improved properties. An optimal library with 35 most preferred mutations from Step 5) was constructed and screened. FIG. 21C shows the PF distribution of all mutants in the optimal library (35-Mut Optimal Lib), Top 3 mutants from original intelligent library (71-Mut Intelligent Lib1), and the pullulanase parent. FIG. 21D shows the PF of the Top 10 mutants from 35-Mut Optimal Lib (N=1) and the average PF of the Top 3 mutants from 71-Mut Intelligent Lib1 (N=80). 35-Mut Optimal Lib significantly outperforms the Top 3 mutants from 71-Mut Intelligent Lib1 in both comparisons. Furthermore, experimentally measured PF values of the Top mutants from 35-Mut Optimal Lib are consistent with the optimal PF values inferred by the Bayesian optimization model.

As exhibited in this Example, intelligent libraries with many mutations and a wide range of mutation rates enable effective mutation interactions. However, such large libraries have huge theoretical size and require enormous amounts of lab screening. Using the combination of surrogate models and searching models, optimal libraries or mutants with performance significantly better than the original data set can be inferred and validated to speed up the discovery of leading protein candidates with preferred mutation interactions and hence supreme properties. Using the disclosed methodology, desired properties of the pullulanase are realized in as few as two rounds of evolution.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to FIG. 3, 4, 5, 6A, 6B, 6C, 6D, 7A, 7B, 7C, 7D, 7E, 7F, 8A, 8B, 8C, 8D, 9A, 9B, 9C, 9D, 9E, 10A, 10B, 11, 12A, 12B, 12C, 13, 14A, 14B, 15, 16A, 16B, 17, 18, 19, 20A, 20B, 21A, 21B, 21C, or 21D. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A computer system comprising:
one or more processors;
a memory; and
one or more programs, wherein the one or more programs are stored in the memory and configured to be executed by the one or more processors, the one or more programs for identifying one or more combinatorial substitutions that affect a first property of a target protein, the one or more programs including instructions for:
A) obtaining an identity of each single point mutation in a first plurality of single point mutations of the target protein, each respective single point mutation in the first plurality of single point mutations defining a corresponding single point substituted protein characterized by a reference sequence for the target protein with the exception of an alteration at a respective independent position within the reference sequence to an amino acid other than that found in the reference sequence;
B) obtaining, for each corresponding point substituted protein defined by the first plurality of single point mutations, a corresponding set of values for a set of properties of the corresponding point substituted protein, wherein the set of properties comprises:
(i) a stability of the corresponding point substituted protein,
(ii) at least one protein formulation property of the corresponding point substituted protein, and
(iii) a determination that the respective single point mutation in the corresponding point substituted protein occurs at a predetermined position that exhibits variability across a plurality of naturally occurring homologs of the target protein;
C) filtering the first plurality of single point mutations to form a second plurality of single point mutations based at least upon each corresponding set of values for the set of properties, wherein the filtering includes determining,
for each corresponding point substituted protein defined by the first plurality of single point mutations,
for each respective property in the set of properties,
whether a value of the respective property in the corresponding set of values for the corresponding point substituted protein satisfies a corresponding threshold value requirement for the respective property, wherein
the corresponding point substituted protein is included in the second plurality of single point mutations when each corresponding threshold value requirement for each property in the set of properties is satisfied, and
the corresponding point substituted protein is not included in the second plurality of single point mutations when any corresponding threshold value requirement of any property in the set of properties is not satisfied;
D) obtaining a corresponding measured value of the first property for each combinatorially substituted protein in a first plurality of combinatorially substituted proteins, wherein each respective combinatorially substituted protein in the first plurality of combinatorially substituted proteins is characterized by the reference sequence for the target protein with the exception of the independent inclusion of two or more single point mutations from the second plurality of single point mutations;
E) training a surrogate model within an N-dimensional space, wherein N is a positive integer of 10 or greater, using at least, for each respective combinatorially substituted protein in the first plurality of combinatorially substituted proteins, the corresponding measured value of the first property in the respective combinatorially substituted proteins against an identity of each single point mutation in the respective combinatorially substituted protein, wherein the model comprises 20 or more parameters and the first plurality of combinatorially substituted proteins comprises 20 or more proteins;
F) using the surrogate model and, for each respective combinatorially substituted protein in the first plurality of combinatorially substituted proteins, the identity of each single point mutation in the respective combinatorially substituted protein, to update a search model; and
G) using the updated search model to identify a second plurality of combinatorially substituted proteins within the N-dimensional space, wherein each respective combinatorially substituted protein in the second plurality of combinatorially substituted proteins is characterized by the reference sequence for the target protein with the exception of independent inclusion of two or more single point mutations from the second plurality of single point mutations.

2. The computer system of claim 1, wherein the at least one protein formulation property is an electrostatic property of the corresponding point substituted protein, a developability index of the corresponding point substituted protein, a solubility of the corresponding point substituted protein, a measure of aggregation of the corresponding point substituted protein, a viscosity of the corresponding point substituted protein, or a combination thereof.

3. The computer system of claim 1, wherein the using G) identifies an optimal range of single point mutations, drawn from the second plurality of single point mutations to incorporate into the target protein.

4. The computer system of claim 1, wherein the set of properties further comprises a post-translational modification that is predicted to occur to the corresponding point substituted protein.

5. The computer system of claim 1, wherein the set of properties further comprises an immunogenicity of the corresponding point substituted protein.

6. The computer system of claim 1, wherein the set of properties further comprises a binding energy of the corresponding point substituted protein.

7. The computer system of claim 1, wherein the first property of the target protein is a solubility of the target protein, an ability of the target protein to carry out an enzymatic activity in a predetermined pH range, aliphatic index, a molecular weight of the target protein, and a charge of the of the target protein.

8. The computer system of claim 1, wherein each combinatorially substituted protein in the first plurality of combinatorially substituted proteins includes three or more, four or more, five or more, or six or more point substitutions.

9. The computer system of claim 1, wherein each combinatorially substituted protein in the first plurality of combinatorially substituted proteins includes between three and fifty point substitutions.

10. The computer system of claim 1, wherein the target protein is an enzyme and the first property is an enzymatic activity of the target protein.

11. The computer system of claim 10, wherein the enzyme is a hydrolase, oxidoreductase, lyase, transferase, ligase or isomerase.

12. The computer system of claim 1, wherein the target protein comprises 50 or more residues, or 100 or more residues.

13. The computer system of claim 1, wherein the stability of the corresponding point substituted protein is determined using one or more crystal structures or atomistic models of the target protein.

14. The computer system of claim 1, wherein the corresponding threshold value for the stability is a stability of the target protein, wherein,
when the corresponding point substituted protein has a stability that is better than the stability of the target protein, the corresponding point substituted protein is included in the second plurality of single point mutations, and
when the corresponding point substituted protein has a stability that is worse than the stability of the target protein, the corresponding point substituted protein is not included in the second plurality of single point mutations.

15. The computer system of claim 1, wherein the corresponding threshold value for the stability is a stability of the target protein, wherein,
when the corresponding point substituted protein has a stability that is at least a threshold percentage or better than the stability of the target protein, the corresponding point substituted protein is included in the second plurality of single point mutations, and
when the corresponding point substituted protein has a stability that is less than a threshold percentage of the stability of the target protein, the corresponding point substituted protein is not included in the second plurality of single point mutations.

16. The computer system of claim 1, wherein the training E) comprises encoding each respective combinatorially substituted protein in the first plurality of combinatorially substituted proteins as an identity of each single point mutation in the respective combinatorially substituted protein in a first dimension, and a position of each single point mutation in the respective combinatorially substituted protein in a second dimension.

17. The computer system of claim 1, wherein the training E) comprises encoding each respective combinatorially substituted protein in the first plurality of combinatorially substituted proteins as an identity of each single point mutation in the respective combinatorially substituted protein in a first dimension, a position of each single point mutation in the respective combinatorially substituted protein in a second dimension, and a plurality of amino acid indices, or a low dimension or latent dimension thereof, for each of the naturally occurring amino acids in a third dimension.

18. The computer system of claim 1, wherein the surrogate model is a support vector regression with RBF kernel, a random forest, XGBoost, a Gaussian Process, a deep neural network, a convolutional neural network, or a recurrent neural network.

19. The computer system of claim 1, wherein the target protein is an enzyme, a co-enzyme, a structural protein, a nutrient protein, a regulatory protein, a defense protein, a transport protein, a storage protein, a contractile protein, or a toxic protein.

20. The computer system of claim 1, wherein the using G) identifies optimal single point mutations in the second plurality of single point mutations to incorporate into the target protein.

21. The computer system of claim 20, wherein the using G) rank orders each single point mutation in the second plurality of single point mutations to incorporate into the target protein.

22. A computer readable storage medium storing one or more programs, the one or more programs comprising instructions, which when executed by an electronic device with one or more processors and a memory cause the electronic device to identify one or more combinatorial substitutions that affect a first property of a target protein by a method comprising:
A) obtaining an identity of each single point mutation in a first plurality of single point mutations of the target protein, each respective single point mutation in the first plurality of single point mutations defining a corresponding single point substituted protein characterized by a reference sequence for the target protein with the exception of an alteration at a respective independent position within the reference sequence to an amino acid other than that found in the reference sequence;
B) obtaining, for each corresponding point substituted protein defined by the first plurality of single point mutations, a corresponding set of values for a set of properties of the corresponding point substituted protein, wherein the set of properties comprises:
(i) a stability of the corresponding point substituted protein,
(ii) at least one protein formulation property of the corresponding point substituted protein, and
(iii) a determination that the respective single point mutation in the corresponding point substituted protein occurs at a predetermined position that exhibits variability across a plurality of naturally occurring homologs of the target protein;
C) filtering the first plurality of single point mutations to form a second plurality of single point mutations based at least upon each corresponding set of values for the set of properties, wherein the filtering includes determining,
for each corresponding point substituted protein defined by the first plurality of single point mutations,
for each respective property in the set of properties,
whether a value of the respective property in the corresponding set of values for the corresponding point substituted protein satisfies a corresponding threshold value requirement for the respective property, wherein
the corresponding point substituted protein is included in the second plurality of single point mutations when each corresponding threshold value requirement for each property in the set of properties is satisfied, and
the corresponding point substituted protein is not included in the second plurality of single point mutations when any corresponding threshold value requirement of any property in the set of properties is not satisfied;
D) obtaining a corresponding measured value of the first property for each combinatorially substituted protein in a first plurality of combinatorially substituted proteins, wherein each respective combinatorially substituted protein in the first plurality of combinatorially substituted proteins is characterized by the reference sequence for the target protein with the exception of the independent inclusion of two or more single point mutations from the second plurality of single point mutations;
E) training a surrogate model within an N-dimensional space, wherein N is a positive integer of 10 or greater, using at least, for each respective combinatorially substituted protein in the first plurality of combinatorially substituted proteins, the corresponding measured value of the first property in the respective combinatorially substituted proteins against an identity of each single point mutation in the respective combinatorially substituted protein, wherein the model comprises 20 or more parameters and the first plurality of combinatorially substituted proteins comprises 20 or more proteins;
F) using the surrogate model and, for each respective combinatorially substituted protein in the first plurality of combinatorially substituted proteins, the identity of each single point mutation in the respective combinatorially substituted protein, to update a search model; and
G) using the updated search model to identify a second plurality of combinatorially substituted proteins within the N-dimensional space, wherein each respective combinatorially substituted protein in the second plurality of combinatorially substituted proteins is characterized by the reference sequence for the target protein with the exception of independent inclusion of two or more single point mutations from the second plurality of single point mutations.

23. A method comprising:
A) obtaining an identity of each single point mutation in a first plurality of single point mutations of the target protein, each respective single point mutation in the first plurality of single point mutations defining a corresponding single point substituted protein characterized by a reference sequence for the target protein with the exception of an alteration at a respective independent position within the reference sequence to an amino acid other than that found in the reference sequence;
B) obtaining, for each corresponding point substituted protein defined by the first plurality of single point mutations, a corresponding set of values for a set of properties of the corresponding point substituted protein, wherein the set of properties comprises:
(i) a stability of the corresponding point substituted protein,
(ii) at least one protein formulation property of the corresponding point substituted protein, and
(iii) a determination that the respective single point mutation in the corresponding point substituted protein occurs at a predetermined position that exhibits variability across a plurality of naturally occurring homologs of the target protein;
C) filtering the first plurality of single point mutations to form a second plurality of single point mutations based at least upon each corresponding set of values for the set of properties, wherein the filtering includes determining,
for each corresponding point substituted protein defined by the first plurality of single point mutations,
for each respective property in the set of properties,
whether a value of the respective property in the corresponding set of values for the corresponding point substituted protein satisfies a corresponding threshold value requirement for the respective property, wherein
the corresponding point substituted protein is included in the second plurality of single point mutations when each corresponding threshold value requirement for each property in the set of properties is satisfied, and
the corresponding point substituted protein is not included in the second plurality of single point mutations when any corresponding threshold value requirement of any property in the set of properties is not satisfied;
D) obtaining a corresponding measured value of the first property for each combinatorially substituted protein in a first plurality of combinatorially substituted proteins, wherein each respective combinatorially substituted protein in the first plurality of combinatorially substituted proteins is characterized by the reference sequence for the target protein with the exception of the independent inclusion of two or more single point mutations from the second plurality of single point mutations;
E) training a surrogate model within an N-dimensional space, wherein N is a positive integer of 10 or greater, using at least, for each respective combinatorially substituted protein in the first plurality of combinatorially substituted proteins, the corresponding measured value of the first property in the respective combinatorially substituted proteins against an identity of each single point mutation in the respective combinatorially substituted protein, wherein the model comprises 20 or more parameters and the first plurality of combinatorially substituted proteins comprises 20 or more proteins;
F) using the surrogate model and, for each respective combinatorially substituted protein in the first plurality of combinatorially substituted proteins, the identity of each single point mutation in the respective combinatorially substituted protein, to update a search model; and
G) using the updated search model to identify a second plurality of combinatorially substituted proteins within the N-dimensional space, wherein each respective combinatorially substituted protein in the second plurality of combinatorially substituted proteins is characterized by the reference sequence for the target protein with the exception of independent inclusion of two or more single point mutations from the second plurality of single point mutations.

24. The computer system of claim 1, wherein the first plurality of combinatorially substituted proteins in D) has mutation rates configured to allow learning of comprehensive interactions between different mutations.

25. The computer system of claim 1, wherein the search model is Bayesian optimization.
